(19) United States
(12) Patent Application Publication
(10) Pub. No.: US 2025/0034523 A1
(43) Pub. Date: Jan. 30, 2025

(54) METHODS OF GENERATING HUMAN COCHLEAR HAIR CELLS

(71) Applicant: THE TRUSTEES OF INDIANA UNIVERSITY, Bloomington, IN (US)

(72) Inventors: Eri HASHINO, Indianapolis, IN (US); Stephen T. MOORE, Flower Mound, TX (US)

(21) Appl. No.: 18/716,889

(22) PCT Filed: Dec. 9, 2022
(86) PCT No.: PCT/US2022/081292

§ 371 Date: Jun. 5, 2024

Related U.S. Application Data
(60) Provisional application No. 63/287,761, filed on Dec. 9, 2021.

(51) Int. Cl. C12N 5/0793

(52) U.S. Cl. CPC C12N 5/062; C12N 2501/115; C12N 2501/155; C12N 2501/41; C12N 2501/415; C12N 2501/727; C12N 2506/02; C12N 2513/00

(57) ABSTRACT

Provided herein are methods for generation of human cochlear hair cells from pluripotent stem cells. More specifically, provided herein are methods for generating three-dimensional cell organoids comprising inner ear hair cells, sensory neurons and supporting cells from human pluripotent stem cells.

### CROSS-REFERENCE TO RELATED APPLICATIONS

The present application claims priority to U.S. Provisional Patent Application No. 63/287,761 that was filed Dec. 9, 2021, the entire contents of which are hereby incorporated by reference.

### STATEMENT REGARDING FEDERALLY SPONSORED RESEARCH

This invention was made with government support under W81XWH-18-1-0062 awarded by the Defense Advanced Research Projects Agency, and DC015788, and DC013294 awarded by the National Institutes of Health. The government has certain rights in the invention.

### SEQUENCE LISTING

A Sequence Listing accompanies this application and is submitted as an xml file of the sequence listing named “144578_00353.xml” which is 27,822 bytes in size and was created on Dec. 9, 2022. The sequence listing is electronically submitted via Patent Center and is incorporated herein by reference in its entirety.

### FIELD OF THE INVENTION

The disclosed technology is generally directed to methods for directing differentiation of human pluripotent stem cells into cochlear hair cells.

### BACKGROUND OF THE INVENTION

The human inner ear is comprised of the cochlear and vestibular organs, each of which contains two types of structurally distinctive mechanosensitive hair cells. During embryogenesis, the cochlear organ is derived from the most ventral region of the otic vesicle, whereas the vestibular organ is derived from the adjacent more dorsal region. A balance of morphogen gradients during embryogenesis is thought to determine the identity of inner ear end organs. Previous methods to generate inner ear sensory epithelia from aggregates of mouse or human pluripotent stem cells do not produce cochlear cell types, as the derived hair cells bear solely structural and functional properties of native vestibular hair cells.

### BRIEF SUMMARY OF THE INVENTION

In an aspect of the current disclosure, methods of generating human cochlear hair cells are provided. In some embodiments, the methods comprise: (a) culturing PAX2b⁺ otic progenitor cells derived from human pluripotent stem cells in medium comprising an activator of sonic hedgehog for about 5 days; and (b) subsequently culturing the cells of step (a) in medium comprising an activator of sonic hedgehog and a Wnt inhibitor for about 4 days after step (a); (c) further culturing the cells of step (b) for a sufficient amount of time to differentiate the cells into human cochlear hair cells which express one or more of the otic markers of PRESTIN, NR2F1, GATA3, INSM1, HES6, TMPRSS3 or GNG8. In some embodiments, the activator of sonic hedgehog in steps (a) and (b) is purmorphamine. In some embodiments, the concentration of purmorphamine is about 1 nM to about 1 mM. In some embodiments, the Wnt inhibitor is IWP-2. In some embodiments, the concentration of IWP-2 is about 1 nM to about 1 mM. In some embodiments, the otic progenitor cells are cultured with thyroxine for about 50 days, starting about 39 days after the start of step (a In some embodiments, the concentration of thyroxine in the medium is about 250 ng/ml. In some embodiments, a sufficient amount of time is about 89 days after the start of step (a), and wherein the medium does not contain additional agonists or inhibitors about 11 days after the start of step (a). In some embodiments, a sufficient amount of time is about 139 days after the start of step (a), and wherein the medium does not contain additional agonists or inhibitors about 11 days after the start of step (a). In some embodiments, the cochlear hair cells express two or markers selected from PRESTIN, NR2F1, GATA3, and INSM1 about 98 days after the start of step (a). In some embodiments, the cells express PRESTIN, NR2F1, GATA3, and INSM1 about 98 days after the start of step (a). In some embodiments, the cells further express one or more additional markers selected from HES6, TMPRSS3 and GNG8 about 98 days after the start of step (a). In some embodiments, the method results in cells expressing two or more markers selected from PRESTIN, GATA3, INSM1, HES6, TMPRSS3 and GNG8 about 98 days after the start of step (a). In some embodiments, the method results in cells expressing three or more markers selected from PRESTIN, GATA3, INSM1, HES6, TMPRSS3 and GNG8 about 98 days after the start of step (a). In some embodiments, the otic progenitor cells are derived from pluripotent stem cells by the method of: (a) culturing pluripotent stem cells in medium comprising FGF-2, BMP-4, and a TGF-beta inhibitor about 3 days on coated plates; (b) further culturing the cells of step (a) in medium comprising FGF-2, a TGF-beta inhibitor and a BMP-4 inhibitor for about 4 days; (c) further culturing the cells of step (b) in medium comprising a GSK-3 Inhibitor, a BMP-4 inhibitor and FGF-2 for about 4 days; and (d) further culturing the cells of step (c) in medium comprising a GSK-3 inhibitor on coated plates for about 2 days to produce PAX2b⁺ otic progenitor cells. In some embodiments, the pluripotent stem cells are induced pluripotent stem cells or embryonic stem cells. In some embodiments, the concentration of BMP-4 is greater than about 100 μg/ml. In some embodiments, the concentration of BMP-4 is greater than about 500 μg/ml. In some embodiments, the concentration of BMP-4 is about 100 μg/ml to about 1000 μg/ml. In some embodiments, the concentration of BMP-4 is about 500 μg/ml to about 1000 μg/ml.

In another aspect of the current disclosure, methods are provided. In some embodiments, the methods comprise: (a) culturing pluripotent stem cells in medium comprising FGF-2, BMP-4, and SB431542 for about 3 days on coated plates; (b) further culturing the cells of step (a) in medium comprising FGF-2, SB431542 and LDN193189 for about 4 days; (c) further culturing the cells of step (b) in medium comprising CHIR99021, LDN193189, and FGF-2 for about 4 days; (d) further culturing the cells of step (c) in medium comprising CHIR99021 on coated plates for about 2 days to generate PAX2b⁺ progenitor cells. In some embodiments, the methods further comprise: (e) culturing the cells of step (d) in medium comprising CHIR99021 and purmorphamine for about 5 days; (f) further culturing the cells of step (e) in medium comprising CHIR99021, purmorphamine, and IWP-2 for about 4 days; (g) further culturing the cells of step (f) in medium for about at least about 78 days to generate human cochlear hair cells. In some embodiments, the medium in step (g) does not comprise CHIR99021, purmorphamine, or IWP-2.

In another aspect of the current disclosure, further methods are provided. In some embodiments, the methods comprise: (a) culturing pluripotent stem cells in medium comprising FGF-2, BMP-4, and SB431542 for about 3 days on coated plates; (b) further culturing the cells of step (a) in medium comprising FGF-2, SB431542 and LDN193189 for about 4 days; (c) further culturing the cells of step (b) in medium comprising CHIR99021, LDN193189, and FGF-2 for about 4 days; (d) further culturing the cells of step (c) in medium comprising CHIR99021 on coated plates for about 2 days; (e) further culturing the cells of step (d) in medium comprising CHIR99021 and purmorphamine for about 5 days; (f) further culturing the cells of step (e) in medium comprising CHIR99021, purmorphamine, and IWP-2 for about 4 days; (g) further culturing the cells of step (f) in medium for about at least about 78 days to generate human cochlear hair cells. In some embodiments, the medium in step (g) does not comprise CHIR99021, purmorphamine, or IWP-2.

In another aspect of the current disclosure, further methods are provided. In some embodiments, the methods comprise: (a) culturing pluripotent stem cells in medium comprising FGF-2, BMP-4, and SB431542 for about 3 days on coated plates; (b) further culturing the cells of step (a) in medium comprising FGF-2, SB431542 and LDN193189 for about 4 days; (c) further culturing the cells of step (b) in medium comprising CHIR99021, LDN193189, and FGF-2 for about 4 days; (d) further culturing the cells of step (c) in medium comprising CHIR99021 on coated plates for about 2 days; (e) further culturing the cells of step (d) in medium comprising CHIR99021 and purmorphamine for about 5 days; (f) further culturing the cells of step (e) in medium comprising CHIR99021, purmorphamine, and IWP-2 for about 4 days; (g) further culturing the cells of step (f) in medium for about 28 days; (h) further culturing the cells of step (g) in medium comprising thyroxine for about 50 days to generate human cochlear hair cells. In some embodiments, the medium in step (g) does not comprise CHIR99021, purmorphamine, or IWP-2.

In another aspect of the current disclosure, human cochlear hair cells are provided. In some embodiments, the human cochlear hair cells are generated by: (a) culturing PAX2b⁺ otic progenitor cells derived from human pluripotent stem cells in medium comprising an activator of sonic hedgehog for about 5 days; and (b) subsequently culturing the cells of step (a) in medium comprising an activator of sonic hedgehog and a Wnt inhibitor for about 4 days after step (a); (c) further culturing the cells of step (b) for a sufficient amount of time to differentiate the cells into human cochlear hair cells which express one or more of the otic markers of PRESTIN, NR2F1, GATA3, INSM1, HES6, TMPRSS3 or GNG8. In some embodiments, the concentration of purmorphamine is about 1 nM to about 1 mM. In some embodiments, the Wnt inhibitor is IWP-2. In some embodiments, the concentration of IWP-2 is about 1 nM to about 1 mM. In some embodiments, the otic progenitor cells are cultured with thyroxine for about 50 days, starting about 39 days after the start of step (a In some embodiments, the concentration of thyroxine in the medium is about 250 ng/ml. In some embodiments, a sufficient amount of time is about 89 days after the start of step (a), and wherein the medium does not contain additional agonists or inhibitors about 11 days after the start of step (a). In some embodiments, a sufficient amount of time is about 139 days after the start of step (a), and wherein the medium does not contain additional agonists or inhibitors about 11 days after the start of step (a). In some embodiments, the cochlear hair cells express two or markers selected from PRESTIN, NR2F1, GATA3, and INSM1 about 98 days after the start of step (a). In some embodiments, the cells express PRESTIN, NR2F1, GATA3, and INSM1 about 98 days after the start of step (a). In some embodiments, the cells further express one or more additional markers selected from HES6, TMPRSS3 and GNG8 about 98 days after the start of step (a). In some embodiments, the method results in cells expressing two or more markers selected from PRESTIN, GATA3, INSM1, HES6, TMPRSS3 and GNG8 about 98 days after the start of step (a). In some embodiments, the method results in cells expressing three or more markers selected from PRESTIN, GATA3, INSM1, HES6, TMPRSS3 and GNG8 about 98 days after the start of step (a). In some embodiments, the otic progenitor cells are derived from pluripotent stem cells by the method of: (a) culturing pluripotent stem cells in medium comprising FGF-2, BMP-4, and a TGF-beta inhibitor about 3 days on coated plates; (b) further culturing the cells of step (a) in medium comprising FGF-2, a TGF-beta inhibitor and a BMP-4 inhibitor for about 4 days; (c) further culturing the cells of step (b) in medium comprising a GSK-3 Inhibitor, a BMP-4 inhibitor and FGF-2 for about 4 days; and (d) further culturing the cells of step (c) in medium comprising a GSK-3 inhibitor on coated plates for about 2 days to produce PAX2b⁺ otic progenitor cells. In some embodiments, the pluripotent stem cells are induced pluripotent stem cells or embryonic stem cells. In some embodiments, the concentration of BMP-4 is greater than about 100 μg/ml. In some embodiments, the concentration of BMP-4 is greater than about 500 μg/ml. In some embodiments, the concentration of BMP-4 is about 100 μg/ml to about 1000 μg/ml. In some embodiments, the concentration of BMP-4 is about 500 μg/ml to about 1000 μg/ml.

In some embodiments, the human cochlear hair cells are generated by: (a) culturing pluripotent stem cells in medium comprising FGF-2, BMP-4, and SB431542 for about 3 days on coated plates; (b) further culturing the cells of step (a) in medium comprising FGF-2, SB431542 and LDN193189 for about 4 days; (c) further culturing the cells of step (b) in medium comprising CHIR99021, LDN193189, and FGF-2 for about 4 days; (d) further culturing the cells of step (c) in medium comprising CHIR99021 on coated plates for about 2 days to generate PAX2b⁺ progenitor cells. In some embodiments, the methods further comprise: (e) culturing the cells of step (d) in medium comprising CHIR99021 and purmorphamine for about 5 days; (f) further culturing the cells of step (e) in medium comprising CHIR99021, purmorphamine, and IWP-2 for about 4 days; (g) further culturing the cells of step (f) in medium for about at least about 78 days to generate human cochlear hair cells. In some embodiments, the medium in step (g) does not comprise CHIR99021, purmorphamine, or IWP-2.

In some embodiments, the human cochlear hair cells are generated by: (a) culturing pluripotent stem cells in medium comprising FGF-2, BMP-4, and SB431542 for about 3 days on coated plates; (b) further culturing the cells of step (a) in medium comprising FGF-2, SB431542 and LDN193189 for about 4 days; (c) further culturing the cells of step (b) in medium comprising CHIR99021, LDN193189, and FGF-2 for about 4 days; (d) further culturing the cells of step (c) in medium comprising CHIR99021 on coated plates for about 2 days; (e) further culturing the cells of step (d) in medium comprising CHIR99021 and purmorphamine for about 5 days; (f) further culturing the cells of step (e) in medium comprising CHIR99021, purmorphamine, and IWP-2 for about 4 days; (g) further culturing the cells of step (f) in medium for about at least about 78 days to generate human cochlear hair cells. In some embodiments, the medium in step (g) does not comprise CHIR99021, purmorphamine, or IWP-2.

In some embodiments, the human cochlear hair cells are generated by: (a) culturing PAX2b⁺ otic progenitor cells derived from human pluripotent stem cells in medium comprising an activator of sonic hedgehog for about 5 days; and (b) subsequently culturing the cells of step (a) in medium comprising an activator of sonic hedgehog and a Wnt inhibitor for about 4 days after step (a); (c) further culturing the cells of step (b) for a sufficient amount of time to differentiate the cells into human cochlear hair cells which express one or more of the otic markers of PRESTIN, NR2F1, GATA3, INSM1, HES6, TMPRSS3 or GNG8. In some embodiments, the concentration of purmorphamine is about 1 nM to about 1 mM. In some embodiments, the Wnt inhibitor is IWP-2. In some embodiments, the concentration of IWP-2 is about 1 nM to about 1 mM. In some embodiments, the otic progenitor cells are cultured with thyroxine for about 50 days, starting about 39 days after the start of step (a In some embodiments, the concentration of thyroxine in the medium is about 250 ng/ml. In some embodiments, a sufficient amount of time is about 89 days after the start of step (a), and wherein the medium does not contain additional agonists or inhibitors about 11 days after the start of step (a). In some embodiments, a sufficient amount of time is about 139 days after the start of step (a), and wherein the medium does not contain additional agonists or inhibitors about 11 days after the start of step (a). In some embodiments, the cochlear hair cells express two or markers selected from PRESTIN, NR2F1, GATA3, and INSM1 about 98 days after the start of step (a). In some embodiments, the cells express PRESTIN, NR2F1, GATA3, and INSM1 about 98 days after the start of step (a). In some embodiments, the cells further express one or more additional markers selected from HES6, TMPRSS3 and GNG8 about 98 days after the start of step (a). In some embodiments, the method results in cells expressing two or more markers selected from PRESTIN, GATA3, INSM1, HES6, TMPRSS3 and GNG8 about 98 days after the start of step (a). In some embodiments, the method results in cells expressing three or more markers selected from PRESTIN, GATA3, INSM1, HES6, TMPRSS3 and GNG8 about 98 days after the start of step (a). In some embodiments, the otic progenitor cells are derived from pluripotent stem cells by the method of: (a) culturing pluripotent stem cells in medium comprising FGF-2, BMP-4, and a TGF-beta inhibitor about 3 days on coated plates; (b) further culturing the cells of step (a) in medium comprising FGF-2, a TGF-beta inhibitor and a BMP-4 inhibitor for about 4 days; (c) further culturing the cells of step (b) in medium comprising a GSK-3 Inhibitor, a BMP-4 inhibitor and FGF-2 for about 4 days; and (d) further culturing the cells of step (c) in medium comprising a GSK-3 inhibitor on coated plates for about 2 days to produce PAX2b⁺ otic progenitor cells. In some embodiments, the pluripotent stem cells are induced pluripotent stem cells or embryonic stem cells. In some embodiments, the concentration of BMP-4 is greater than about 100 μg/ml. In some embodiments, the concentration of BMP-4 is greater than about 500 μg/ml. In some embodiments, the concentration of BMP-4 is about 100 μg/ml to about 1000 μg/ml. In some embodiments, the concentration of BMP-4 is about 500 μg/ml to about 1000 μg/ml.

In another aspect of the current disclosure, organoids are provided. In some embodiments, the organoids comprise a cochlear hair cell (a) culturing PAX2b⁺ otic progenitor cells derived from human pluripotent stem cells in medium comprising an activator of sonic hedgehog for about 5 days; and (b) subsequently culturing the cells of step (a) in medium comprising an activator of sonic hedgehog and a Wnt inhibitor for about 4 days after step (a); (c) further culturing the cells of step (b) for a sufficient amount of time to differentiate the cells into human cochlear hair cells which express one or more of the otic markers of PRESTIN, NR2F1, GATA3, INSM1, HES6, TMPRSS3 or GNG8. In some embodiments, the concentration of purmorphamine is about 1 nM to about 1 mM. In some embodiments, the Wnt inhibitor is IWP-2. In some embodiments, the concentration of IWP-2 is about 1 nM to about 1 mM. In some embodiments, the otic progenitor cells are cultured with thyroxine for about 50 days, starting about 39 days after the start of step (a In some embodiments, the concentration of thyroxine in the medium is about 250 ng/ml. In some embodiments, a sufficient amount of time is about 89 days after the start of step (a), and wherein the medium does not contain additional agonists or inhibitors about 11 days after the start of step (a). In some embodiments, a sufficient amount of time is about 139 days after the start of step (a), and wherein the medium does not contain additional agonists or inhibitors about 11 days after the start of step (a). In some embodiments, the cochlear hair cells express two or markers selected from PRESTIN, NR2F1, GATA3, and INSM1 about 98 days after the start of step (a). In some embodiments, the cells express PRESTIN, NR2F1, GATA3, and INSM1 about 98 days after the start of step (a). In some embodiments, the cells further express one or more additional markers selected from HES6, TMPRSS3 and GNG8 about 98 days after the start of step (a). In some embodiments, the method results in cells expressing two or more markers selected from PRESTIN, GATA3, INSM1, HES6, TMPRSS3 and GNG8 about 98 days after the start of step (a). In some embodiments, the method results in cells expressing three or more markers selected from PRESTIN, GATA3, INSM1, HES6, TMPRSS3 and GNG8 about 98 days after the start of step (a). In some embodiments, the otic progenitor cells are derived from pluripotent stem cells by the method of: (a) culturing pluripotent stem cells in medium comprising FGF-2, BMP-4, and a TGF-beta inhibitor about 3 days on coated plates; (b) further culturing the cells of step (a) in medium comprising FGF-2, a TGF-beta inhibitor and a BMP-4 inhibitor for about 4 days; (c) further culturing the cells of step (b) in medium comprising a GSK-3 Inhibitor, a BMP-4 inhibitor and FGF-2 for about 4 days; and (d) further culturing the cells of step (c) in medium comprising a GSK-3 inhibitor on coated plates for about 2 days to produce PAX2b⁺ otic progenitor cells. In some embodiments, the pluripotent stem cells are induced pluripotent stem cells or embryonic stem cells. In some embodiments, the concentration of BMP-4 is greater than about 100 μg/ml. In some embodiments, the concentration of BMP-4 is greater than about 500 μg/ml. In some embodiments, the concentration of BMP-4 is about 100 μg/ml to about 1000 μg/ml. In some embodiments, the concentration of BMP-4 is about 500 μg/ml to about 1000 μg/ml. In some embodiments, the organoids comprise a cochlear hair cell generated by: (a) culturing pluripotent stem cells in medium comprising FGF-2, BMP-4, and SB431542 for about 3 days on coated plates; (b) further culturing the cells of step (a) in medium comprising FGF-2, SB431542 and LDN193189 for about 4 days; (c) further culturing the cells of step (b) in medium comprising CHIR99021, LDN193189, and FGF-2 for about 4 days; (d) further culturing the cells of step (c) in medium comprising CHIR99021 on coated plates for about 2 days to generate PAX2b⁺ progenitor cells. In some embodiments, the methods further comprise: (e) culturing the cells of step (d) in medium comprising CHIR99021 and purmorphamine for about 5 days; (f) further culturing the cells of step (e) in medium comprising CHIR99021, purmorphamine, and IWP-2 for about 4 days; (g) further culturing the cells of step (f) in medium for about at least about 78 days to generate human cochlear hair cells. In some embodiments, the medium in step (g) does not comprise CHIR99021, purmorphamine, or IWP-2.

In some embodiments, the organoids comprise a cochlear hair cell generated by: (a) culturing pluripotent stem cells in medium comprising FGF-2, BMP-4, and SB431542 for about 3 days on coated plates; (b) further culturing the cells of step (a) in medium comprising FGF-2, SB431542 and LDN193189 for about 4 days; (c) further culturing the cells of step (b) in medium comprising CHIR99021, LDN193189, and FGF-2 for about 4 days; (d) further culturing the cells of step (c) in medium comprising CHIR99021 on coated plates for about 2 days; (e) further culturing the cells of step (d) in medium comprising CHIR99021 and purmorphamine for about 5 days; (f) further culturing the cells of step (e) in medium comprising CHIR99021, purmorphamine, and IWP-2 for about 4 days; (g) further culturing the cells of step (f) in medium for about at least about 78 days to generate human cochlear hair cells. In some embodiments, the medium in step (g) does not comprise CHIR99021, purmorphamine, or IWP-2.

In some embodiments, the organoids comprise a cochlear hair cell generated by: (a) culturing PAX2b⁺ otic progenitor cells derived from human pluripotent stem cells in medium comprising an activator of sonic hedgehog for about 5 days; and (b) subsequently culturing the cells of step (a) in medium comprising an activator of sonic hedgehog and a Wnt inhibitor for about 4 days after step (a); (c) further culturing the cells of step (b) for a sufficient amount of time to differentiate the cells into human cochlear hair cells which express one or more of the otic markers of PRESTIN, NR2F1, GATA3, INSM1, HES6, TMPRSS3 or GNG8. In some embodiments, the concentration of purmorphamine is about 1 nM to about 1 mM. In some embodiments, the Wnt inhibitor is IWP-2. In some embodiments, the concentration of IWP-2 is about 1 nM to about 1 mM. In some embodiments, the otic progenitor cells are cultured with thyroxine for about 50 days, starting about 39 days after the start of step (a In some embodiments, the concentration of thyroxine in the medium is about 250 ng/ml. In some embodiments, a sufficient amount of time is about 89 days after the start of step (a), and wherein the medium does not contain additional agonists or inhibitors about 11 days after the start of step (a). In some embodiments, a sufficient amount of time is about 139 days after the start of step (a), and wherein the medium does not contain additional agonists or inhibitors about 11 days after the start of step (a). In some embodiments, the cochlear hair cells express two or markers selected from PRESTIN, NR2F1, GATA3, and INSM1 about 98 days after the start of step (a). In some embodiments, the cells express PRESTIN, NR2F1, GATA3, and INSM1 about 98 days after the start of step (a). In some embodiments, the cells further express one or more additional markers selected from HES6, TMPRSS3 and GNG8 about 98 days after the start of step (a). In some embodiments, the method results in cells expressing two or more markers selected from PRESTIN, GATA3, INSM1, HES6, TMPRSS3 and GNG8 about 98 days after the start of step (a). In some embodiments, the method results in cells expressing three or more markers selected from PRESTIN, GATA3, INSM1, HES6, TMPRSS3 and GNG8 about 98 days after the start of step (a). In some embodiments, the otic progenitor cells are derived from pluripotent stem cells by the method of: (a) culturing pluripotent stem cells in medium comprising FGF-2, BMP-4, and a TGF-beta inhibitor about 3 days on coated plates; (b) further culturing the cells of step (a) in medium comprising FGF-2, a TGF-beta inhibitor and a BMP-4 inhibitor for about 4 days; (c) further culturing the cells of step (b) in medium comprising a GSK-3 Inhibitor, a BMP-4 inhibitor and FGF-2 for about 4 days; and (d) further culturing the cells of step (c) in medium comprising a GSK-3 inhibitor on coated plates for about 2 days to produce PAX2b⁺ otic progenitor cells. In some embodiments, the pluripotent stem cells are induced pluripotent stem cells or embryonic stem cells. In some embodiments, the concentration of BMP-4 is greater than about 100 μg/ml. In some embodiments, the concentration of BMP-4 is greater than about 500 μg/ml. In some embodiments, the concentration of BMP-4 is about 100 μg/ml to about 1000 μg/ml. In some embodiments, the concentration of BMP-4 is about 500 μg/ml to about 1000 μg/ml.

In another aspect of the current disclosure, kits, platforms, and systems are provided. In some embodiments, the kits, systems or platforms comprise: (a) an activator of sonic hedgehog; and (b) a Wnt inhibitor. In some embodiments, the kit, system, or platforms further comprise: (c) FGF-2; (d) a TGF-beta inhibitor; (e) a BMP-4 inhibitor; and (f) a GSK-3 Inhibitor. In some embodiments, the kits, systems, or platforms further comprise: (g) thyroxine. In some embodiments, the kits, systems, or platforms further comprise: (h) induced pluripotent stem cells or embryonic stem cells.

### BRIEF DESCRIPTION OF THE DRAWINGS

FIGS. 1a, 1b-11. PAX2-2A-nGFP/POU4F3-2A-ntTomato (PAX2^{nG}/POU4F3^{nT}) multiplex reporter hESCs faithfully recapitulate otic progenitor and hair cell differentiations in inner ear organoids. a, Schematic illustrations of the PAX2-2A-nGFP and POU4F3-2A-ntdTomato CRISPR design. Two pairs of 1 kb homology arms were generated by PCR to flank the stop codon of the otic-predominant PAX2b splice variant and the sole splice variant of POU4F3. The PAX2 construct contained a floxed PGK-Puromycin cassette included for positive selection of correctly targeted clones and was subsequently removed by CRE-recombination following transfection with a CRE recombinase-expression vector. The POU4F3 construct contained a FLPo-flanked PGK-Puromycin cassette that was subsequently removed following transfection with a FLPo recombinase-expression vector. The viral p2A sequence was included to generate separate gene products from a polycistronic mRNA transcript. The nuclear localization sequences were included for properly localized visualization. Single guide RNAs were used to maximize insertion efficiency and minimize off-target activity. b, Schematic of PAX2^{nG} and POU4F3^{nT} reporter expression during inner ear organoid development. c-f, Live images of whole aggregates containing multiple developing inner ear organoids show the spatio-temporal progression of PAX2^{nG}ì reporter expression and early morphogenesis of PAX2⁺ epithelium. g-h, Representative images of hESC-derived aggregates showing PAX2^{nG}+ epithelium organized into vesicles that co-express the otic-specific marker FBXO2, but devoid of POU4F3^{nT} expression. i-i′, Live images of late-stage (D96) aggregates showing intense POU4F3^{nT}+ puncta localized to epithelial vesicles. j-l, POU4F3^{nT}+ cells in inner ear organoids also express the hair cell markers MYO7A, ATOH1 and SOX2, and are located on the luminal surface of SOX2+ supporting epithelia. Scale bars, 200 μm (c-g, h, i, i′), 50 μm (g′, h′), 10 μm (j-l).

FIG. 2a-2c. Optimization of inner ear organoid derivation protocol. a, Schematic comparison of the inventors' original vs. optimized protocol. b, Live images of whole cell aggregates containing inner ear organoids derived from the inventors PAX2G/POU4F3^{nT} multiplex reporter hESC line cultured under the original vs. optimized protocol. c, Quantitative comparison of culture outcomes in optimized vs. original culture protocol. n=20 (green histograms), 13 (red histogram) biological samples from separate experiments per group. Welch's two-sided 1-test ***P=0.000132, ****P=0.000013 (#cysts) P=0.000001 (diameter) Scale bars, 200 μm.

FIGS. 3a, 3b, 3c, 3d, 3e, 3f. PUR+IWP2 treatment promotes ventralization of otic progenitors in human inner ear organoids. a, Schematic illustration of known ventralization and dorsalization signals during mouse inner ear development and application of this principle to the human inner ear organoid system. b-d, D20 scRNA-seq analysis of FACS-sorted PAX2^{nG}+ otic progenitors in human inner ear organoids. UMAP projections of otic progenitors from PUR+IWP2, PUR and CTRL samples (b). Feature plots demonstrate that dorsal otic markers are predominantly expressed in PUR and CTRL otic progenitors, while ventral otic markers and SHH signaling components are confined largely to PUR+IWP2 cells. Consistent with this, the volcano plot (c) shows differentially expressed dorsal and ventral otic marker genes between PUR+IWP2 and CTRL otic progenitors. Gene-set enrichment analysis of genes upregulated in the PUR+IWP2 and CTRL otic progenitors (above and below 0 in the bubble plot, respectively) (d) shows genes associated with posttranscriptional regulation of gene expression, chromatin modifications and Hedge Hog signaling are enriched in ventralized otic progenitors in inner ear organoids. (e) Representative images of D25 samples show notably higher expression of NR2F1 and SUIF1 in PUR+IWP2 organoids vs. CTRL organoids. (f) Quantitative analysis of vesicles co-expressing PAX2 and NR2F1 (or SULF1) in PUR+IWP2 vs. CTRL organoids; n=8 biological samples from separate experiments per group; Welch's two-sided 1-test *P=0.0013 (NR2F1), *P=0.0089 (SULF1); values are mean±SEM. Scale bars, 200 μm.

FIG. 4a, 4b, 4c, 4d, 4e, 4f, 4g. POU4F3^{nT}+ cells in ventralized inner ear organoids express cochlear hair cell markers. a-d, UMAP projections of POU4F-3^{nT}+ cells isolated from D109 PUR+IWP2 and CTRL inner ear organoids (a). Feature plots show differential expression of known cochlear and vestibular marker genes in annotated hair cell populations (b). A volcano plot (c) confirms differentially expressed cochlear and vestibular hair cell marker genes between PUR+IWP2 and CTRL hair cells. Additionally, previously unrecognized genes, such as NR2F1, TMPRSS3, CD164L2, ZBBX and SKOR1, are differentially expressed between PUR+IWP2 and CTRL hair cells. Heat map showing gene expression across clusters (d). e, Representative immunohistochemistry validates differential expression of NR2F1 and GATA3 between PUR+IWP2 and CTRL inner ear organoids. f, Comparison of the percentage of NR2F1- or GATA3-positive hair cells and supporting cells in PUR+IWP2 vs. CTRL inner ear organoids; n=9 biological samples from separate experiments; Welch's two-sided 1-test ****P<0.000001; values are mean±SEM. g, NR2F1 and GATA3 are expressed in both outer and inner hair cells in the human cochlea at GW18. Scale bars, 20 μm (e, g).

FIGS. 5a-50, 5p, 5q. Hair cells derived from ventralized organoids exhibit structural properties of cochlear hair cells. a-h′, Scanning electron micrographs of PUR+IWP2 hair bundles (a-b, f-g) reveal relatively short stereocilia organized into concave rows of increasing height and diameter characteristic of a cochlear hair cell phenotype. In contrast, scanning electron micrographs of CTRL hair bundles (c-e, h-h′) reveal elongated stereocilia organized into convex rows that are of equivalent diameter characteristic of native vestibular hair cells. i-k′, Confocal microscopic images of PUR+IWP2-treated hair cells (i-i′) reveal short F-actin hair bundles and rectangular soma with basally-positioned nuclei, whereas those of CTRL hair cells (j-k′) reveal elongated F-actin hair bundles and an often-bulbous or flask-shaped soma. CTRL hair cells retain vestibular morphology even at D200 (j). p-q, Quantitative analysis of the stereocilia height and the diameter of individual stereocilia in PUR+IWP2 (l-m) vs. CTRL hair cells (j-k′); n=50 biological samples from separate experiments; Welch's two-sided 1-test ****P<0.00001; values are mean±SEM. Scale bars, 10 μm (c, i, j, k), 1 μm (a, b, d-h, k′).

FIGS. 6a, 6b, 6c, 6d, 6e, 6f, 6g, 6h. A subpopulation of hair cells derived from ventralized organoids express PRESTIN and exhibit voltage-gated currents characteristic of cochlear outer hair cells. a-b, PRESTIN+ hair cells increase over time in PUR+IWP2 inner ear organoids. Representative immunohistochemistry for PRESTIN in PUR+IWP2 samples at D102, -150, and -200, along with the comparison of the percentage of hair cells expressing PRESTIN among the different age groups, reveals an increasing number of hair cells expressing membranous PRESTIN over time in culture. In contrast, PRESTIN is undetectable in CTRL hair cells at D110 or -200. c, Live image of a tdTomato-positive sample cut by a diamond knife. d-h, Voltage-gated currents in hESC-derived hair cells. Typical whole cell current responses (top traces) to the voltage step protocol (bottom traces) in type A (d) and type B (e) cells. Average steady-state current amplitude at the end of voltage step in type A (f) and type B (h) cells. The peak amplitude of the negative inward current (g). All data are shown as Mean±Standard Error. Age of the cells: d138-d164. Scale bars, 10 μm (a), 200 μm (c).

FIG. 7a-7c. Thyroxine treatment increases the number of PRESTIN+HCs in cochlear organoids. (a) The number of Prestin positive HCs increases when organoids are treated with 250 ng/ml Thyroxine. (b) Quantification of Prestin+HCs over time in Thyroxine-treated and untreated organoids. (c) Thyroxine-treated HCs downregulate the immature HC marker SOX2, whereas SOX2 expression is maintained in SCs, recapitulating the events of cochlear maturation.

FIG. 8a, 8b, 8c 8d, 8e, 8f, 8g. Generation and validation of a PAX2-2A-nGFP (PAX2^{nG}) reporter hESC line. a-b, Schematic of PAX2 isoforms (a) and RT-PCR data showing that PAX2b is the most abundant isoform expressed in stem cell-derived inner ear organoids (b). c-d, PCR amplification using primer sets shown in (FIG. 1a) demonstrated bi-allelic insertion of the 2A-nGFP cassette at the PAX2 locus (c), which was confirmed by Sanger sequencing (d). e, Immunofluorescence of undifferentiated PAX2^{nG} (hESCs reveals the expression of multiple pluripotency markers and absence of constitutive PAX2^{nG}ì reporter expression. f, Representative immunohistochemistry of sectioned PAX2^{nG}ì hESC-derived inner ear organoids reveals expression of PAX2^{nG} localized to epithelial vesicles that co-express otic markers PAX8 by D20, as well as SOX10 and FBXO2 by D25. Expression of PAX2^{nG} is sustained and can be detected by live imaging at D70. SOX2/MYO7A+ hair cells can be detected on the luminal surface of PAX2^{nG}+ vesicles at D70. g, Sequencing results for the top 10 predicted off-target CRISPR sites reveals no insertions or deletions in the surrounding loci. Scale bars, 50 μm (e), 200 μm (f).

FIG. 9a, 9b, 9c-9f″, 9g, 9h. Generation and validation of a PAX2-2A-nGFP/POU4F3-2A-ntdTomato (PAX2^{nG}/POU4F3^{nT}) reporter hESC line. a-b, PCR amplification of the POU4F3 locus reveals bi-allelic insertion of the 2A-ntdTomato reporter cassette (a). Sanger sequencing reveals correct insertion and orientation of the reporter cassette immediately downstream of the POU4F3 stop codon (b). c-d, POU4F3^{nT} reporter expression is confined to cells on the luminal surface of vesicles at D60 and -100. e, Fixed cell suspension of dissociated POU4F3^{nT}+ cells isolated from D80 inner ear organoids reveals tdTomato+ nuclei and F-actin+ membranes and stereocilia. f-f″, Immunohistochemistry of D110 inner ear organoids derived from POU4F3^{nT} hESCs reveals that tdTomato+ puncta label the nuclei of PCP4+ hair cells and perfectly colocalize with antibody-labeled POU4F3. g, Sequencing results for the top 10 predicted off-target CRISPR sites reveal no insertions or deletions in the surrounding loci. h, The PAX2^{nG}/POU4F3^{nT} hESC line exhibits normal karyotyping results. Scale bars, 200 μm (c, d), 10 μm (e), 100 μm (f).

FIG. 10a, 1b, 10c. Optimization of BMP4 concentration for induction of non-neural ectoderm and downstream cochlear organoid formation. a. Representative live images of cell aggregates over time under various concentrations of recombinant BMP4 applied on day 0. Cultures were otherwise maintained in conditions described for the inner ear and cochlear differentiation protocol. b, Quantification of the GFP+ area fraction in live images at day 20; n=4 aggregates per condition; One-way ANOVA, Dunnet's multiple comparisons test, *P<0.01. c, Population proportions of TdTomato expressing cell aggregates on day 55. Scale bars, 200 μm (a).

FIG. 11a-11g. scRNA-seq analysis of PAX2^{nG} cells in D20 CTRL, PUR, and PUR+IWP2 inner ear organoids. a, FACS gating strategy used to isolate PAX2^{nG}ì+ cells from whole aggregates. b, Cell clusters were generated by Seurat and visualized using UMAP. c, Dot plot showing the relative expression of marker genes within annotated clusters. d, Feature plots show canonical markers of otic progenitors, neuroblasts, and cycling cells. e, Colored cells show the distribution of conditions within each cluster. A stacked histogram shows the composition of each cluster by condition. f, Representative immunohistochemistry of D25 organoid sections reveals differential expression of OTX2 and DLX3 between conditions. g, Quantitative comparison of OTX2 and DLX3 expression between PUR+IWP2 and CTRL conditions by immunohistochemistry; n=5 biological samples from separate experiments per group; Welch's two-sided t-test **P<. 001; ***P<0.0001; values are mean±SEM. Scale bars, 200 μm

FIG. 12a, 12b, 12c-12h, 12i, 12j. Protein Kinase A inhibition fails to promote hair cell differentiation. a, Representative immunohistochemistry of the regionally expressed otic markers OTX2, GATA3, and DLX3 for D25 inner ear organoids treated with or without 10 μM H89 alone or in combination with PUR or PUR+IWP2. b, Schematic illustration of the proposed role of H89 in the SHH pathway. c, Live image of a D102 cell aggregate treated with H89 and PUR shows a single POU4F3^{nT}+ inner ear organoid. d, Representative image of a D102 PUR+H89-treated aggregate showing a small number of POU4F3^{nT}+ puncta. e, Immunohistochemistry of a D102 inner ear organoid treated with H89+PUR shows MYO7A+ hair cells with POU4F3^{nT}+ nuclei on the luminal surface of SOX2⁺ epithelium. f, Confocal image of a hair cell in a H89+PUR-treated organoid shows detectible expression of the cochlear outer hair cell marker LMOD3. g-h, Immunohistochemistry of D102 H89+PUR-treated inner ear organoids stained with phalloidin reveals hair bundles with vestibular-like length and morphology. i, Quantitative comparison of OTX2, GATA3 and DLX3 expression among different treatment groups; n=5 biological samples from separate experiments per group; *P<0.01; values are mean #SEM. j, Quantitative comparison of the percentage of tdTomato expressing aggregates (left y-axis), and total tdTomato positive area per aggregate (right y-axis) among different treatment groups; n=12 biological samples from separate experiments per group; Welch's two-sided 1-test *P<0.01; ns, not significant; values are mean±SEM. Scale bars, 200 μm (a-d), 20 μm (e-g), 5 μm (h).

FIG. 13a, 13b, 13c, 13d, 13g. scRNA-seq analysis of FACS-sorted POU4F3^{nT}+ cells in D80 CTRL and PUR+IWP2 inner ear organoids. a, UMAP projections showing annotated clusters of POU4F3^{nt}+ cells. b, Feature plots showing the distributions of inner ear and neural marker genes. c, Dot plot showing the relative expression of marker genes within annotated clusters. d, Volcano plot depicting differentially expressed genes between PUR+IWP2 and CTRL hair cells shown in magenta and blue, respectively. g, Feature plots with accompanying violin plots showing the distribution of cochlear and vestibular gene expression across the cluster map and between PUR+IWP2 and CTRL hair cells shown in magenta and blue, respectively.

FIG. 14a, 14b, 14c, 14d, 14e, 14f, 14g, 14h. scRNA-seq analysis of FACS-sorted POU4F3^{nT}+ cells in D109 CTRL and PUR+IWP2 inner ear organoids. a, FACS gating strategy used to isolate POU4F3^{nT}+ cells from dissociated Day-109 inner ear organoids in PUR+IWP2 and CTRL conditions. b, Feature plots showing the distributions of marker genes across the cluster map. c, Dual feature plot showing the expression patterns of GATA3 and MEIS2. d, Pseudo-time analysis of POUF4F3^{nT}+ cells in the PUR+IWP2 condition reveals a branched trajectory with LGR5+ cells adopting either a hair cell-like or supporting cell-like fate. e, Violin plots depicting the distribution of cochlear and vestibular marker genes between PUR+IWP2 and CTRL conditions shown in magenta and blue, respectively. f, Gene-set enrichment analysis of genes upregulated in the PUR+IWP2 and CTRL conditions (above and below 0 in the bubble plot, respectively) shows that gene sets associated with voltage-gated cation channel activities are upregulated in hair cells of PUR+IWP2 inner ear organoids, when compared to CTRL organoids. g, Representative immunohistochemistry showing differential expression of INSM1 and NDGR1 between PUR+IWP2 and CTRL inner ear organoids. h, Quantitative comparison of the expression levels of INSM1 and NDGR1 in PUR+IWP2 and CTRL inner ear organoids; n=5 biological samples from separate experiments per group; Welch's two-sided 1-test*P=0.001468, P<0.000012; values are mean±SEM. Scale bars, 20 μm.

FIG. 15a-15g, 15h-15m′. Hair cells in PUR+IWP2 and CTRL inner ear organoids exhibit distinctive hair bundle morphology. a-c″′, Scanning electron micrographs of hair bundles from PUR+IWP2 treated cells shows developmental progression of hair bundle organization including the assembly of tip links. d-d′, Scanning electron micrographs showing increasing diameter of stereocilia on the apical surface of a PUR+IWP2 hair cell. e-f, Confocal microscopic images showing short F-actin+ stereocilia on the apical surface of PUR+IWP2 treated hair cells at D110 and -200. g, TUJ1+ neurite processes contacting PUR+IWP2 hair cells at D200. h-k, Scanning electron micrographs of CTRL hair bundles reveal long pointed morphologies with consistent-diameter stereocilia within each hair bundle. l-l′, Confocal microscopic images show long, pointed F-actin+ stereocilia on the surface of CTRL hair cells. m-m′, TUJ1+ neurite processes contacting CTRL hair cells at Day-200. Scale bars, 1 μm (a, b, c, d), 500 nm (a′, c′, d′, j, k), 100 nm (c″, c′″), 10 μm (e′, h, i, l′, m′), 20 μm (e, f, g, l, m).

FIG. 16a-16e″. Timeline of human inner ear organoid development closely mirrors that of human fetal inner ear development. Schematic depicting the timings of developmental events during native human cochlear development and those during human cochlear organoid development as observed in the present study. a-c″, Low-magnification modiolar section of a human GW13 cochlea (a) and immunofluorescence images showing the presence of inner and outer hair cells (b-b′) and the lack of PRESTIN expression in cochlear hair cells (c-c″) at this stage. d-e″, Low-magnification modiolar section of a human GW 18 cochlea (d) and immunofluorescence images showing membranous PRESTIN expression in outer hair cells (e-e″). Scale bars, 1000 μm (a, d), 50 μm (b, c, e), 20 μm (b′, c′, e′).

FIG. 17. Exemplary timeline of inner ear organoid protocol. The exemplary protocol depicted in FIG. 17 begins with aggregated stem cells at D0 and results in Pax2b⁺ otic precursors at D11-D13 and PRESTIN cochlear hair cells after about 100 days in culture.

### DETAILED DESCRIPTION OF THE INVENTION

The inventors have developed a next-generation organoid system to generate cochlear hair cells from human pluripotent stem cells. This in vitro model system may be used to more properly study hearing disorders and therapies for treating diseases and disorders associated with dysfunction or loss of cochlear hair cells, including deafness. The inventors have developed a method for directing differentiation of human pluripotent stem cells into cochlear hair cells that can transmit auditory sensation. The inventors had previously developed methods to generate inner ear sensory epithelia from aggregates of mouse or human pluripotent stem cells, but a major limitation of this system was the absence of cochlear cell types, as derived hair cells bear solely structural and functional properties of native vestibular hair cells. Here, a next-generation organoid system to generate cochlear hair cells from human pluripotent stem cells is provided that can be used for both in vitro model systems to more properly study hearing disorders and therapeutic modalities.

Methods of Deriving Cochlear Hair Cells In Vitro from Pluripotent Stem Cells

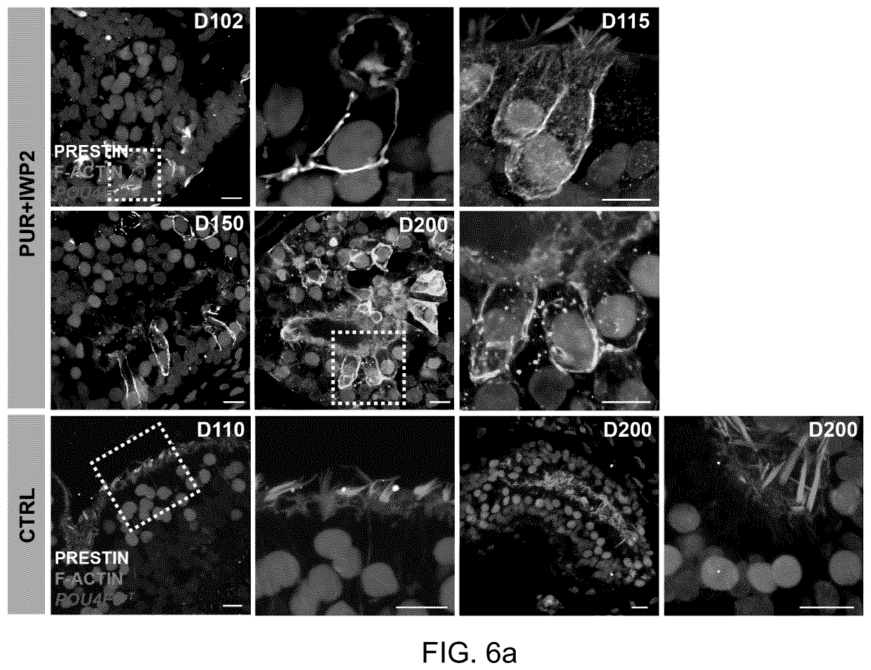

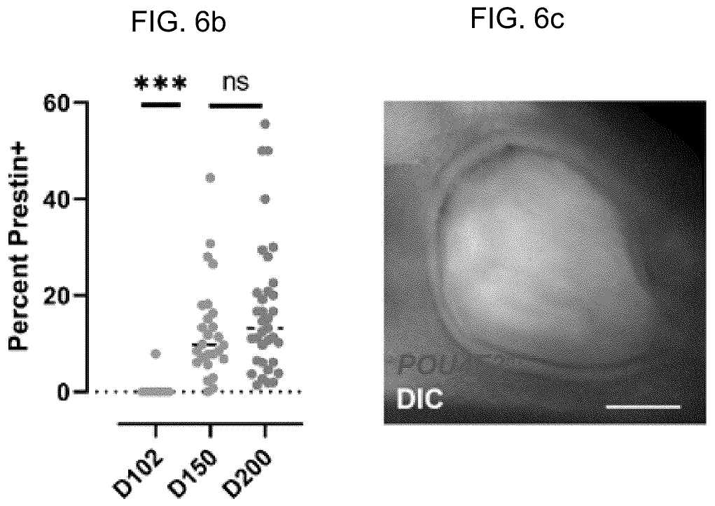

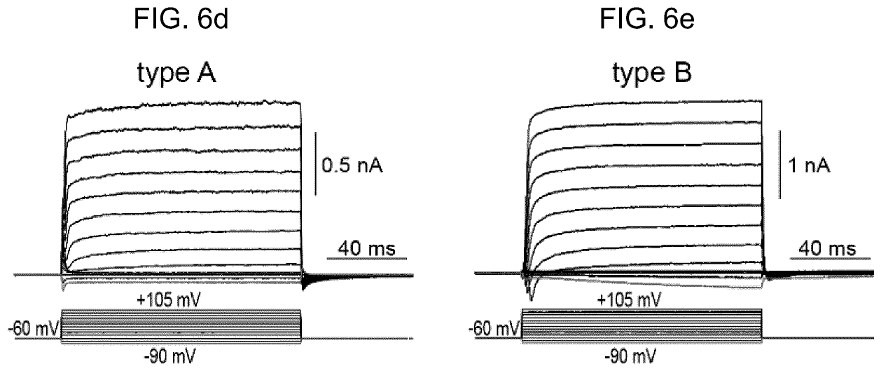

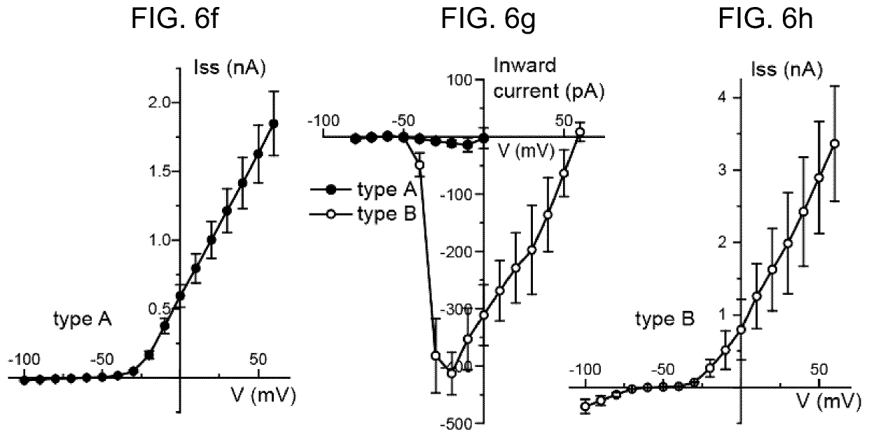

In one aspect of the current disclosure, methods of generating human cochlear hair cells are provided. In some embodiments, the methods of generating human cochlear hair cells comprise (a) culturing PAX2 otic progenitor cells derived from human pluripotent stem cells in medium comprising an activator of sonic hedgehog for at least 3 to 5 days; and (b) subsequently culturing the cells of step (a) in medium comprising an activator of sonic hedgehog and a Wnt inhibitor for a sufficient amount of time to differentiate the cells into human cochlear hair cells which express one or more of the cochlear hair cell markers of GATA3, NR2F1, INSM1 or PRESTIN. Preferably, the human cochlear hair cells can transmit auditory sensation, see, FIGS. 6d-6f.

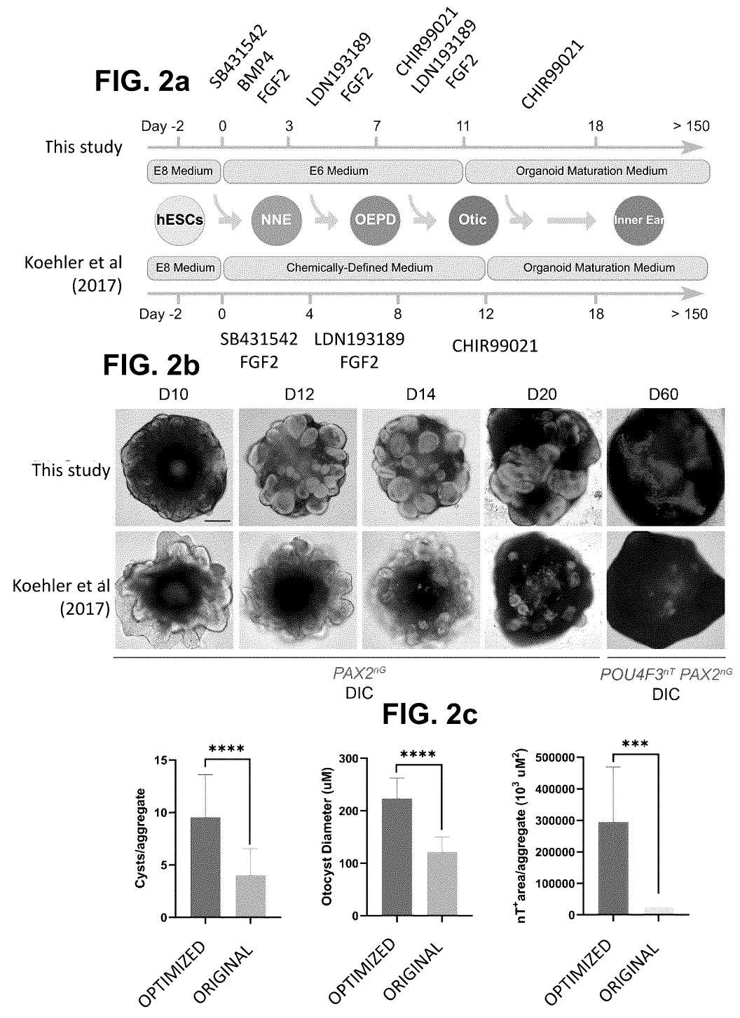

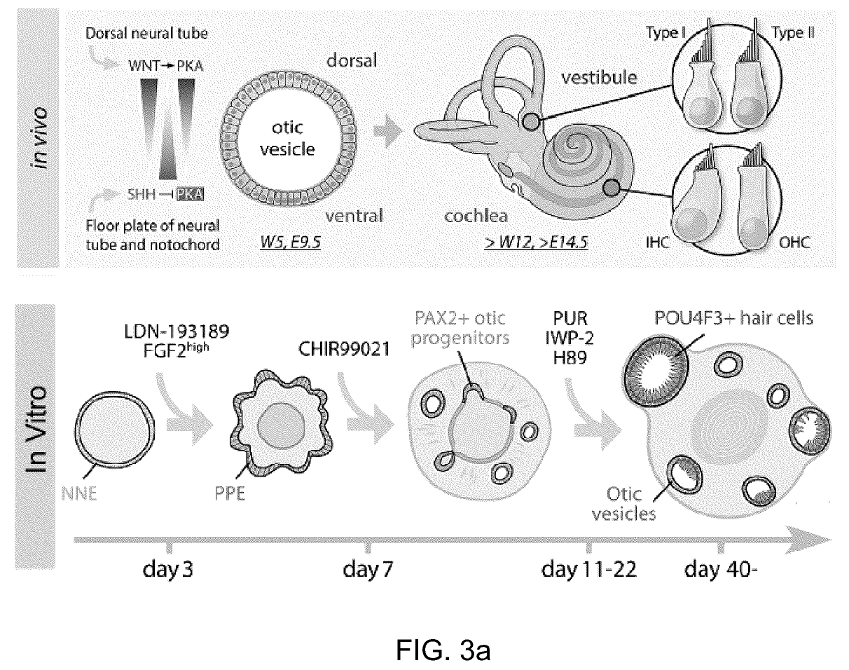

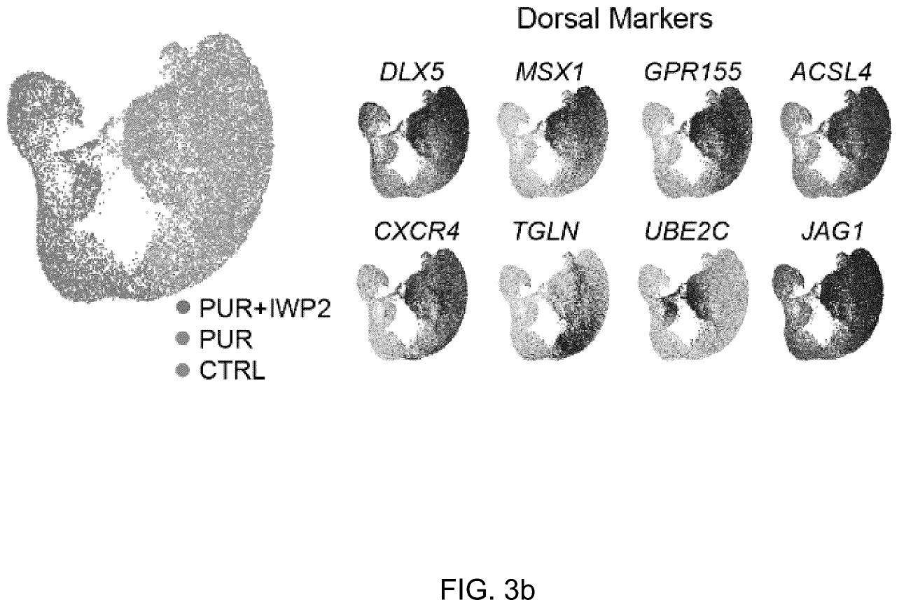

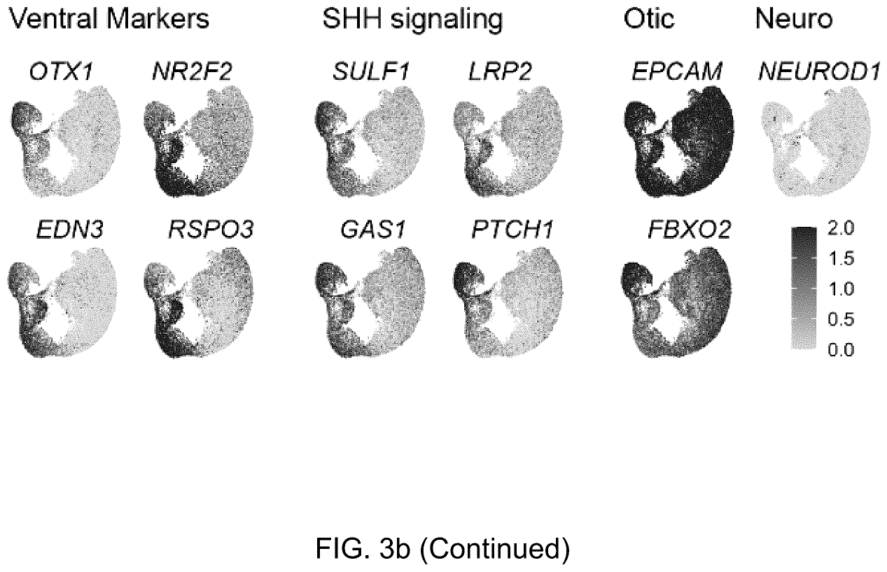

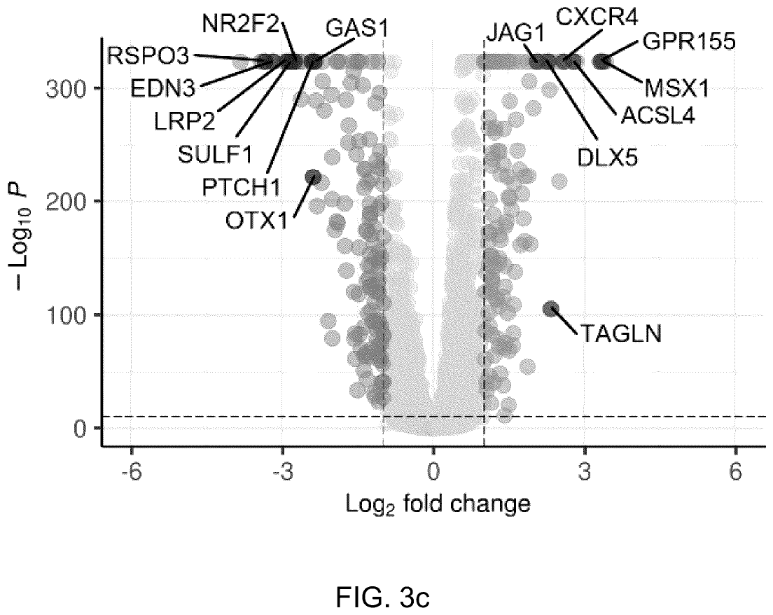

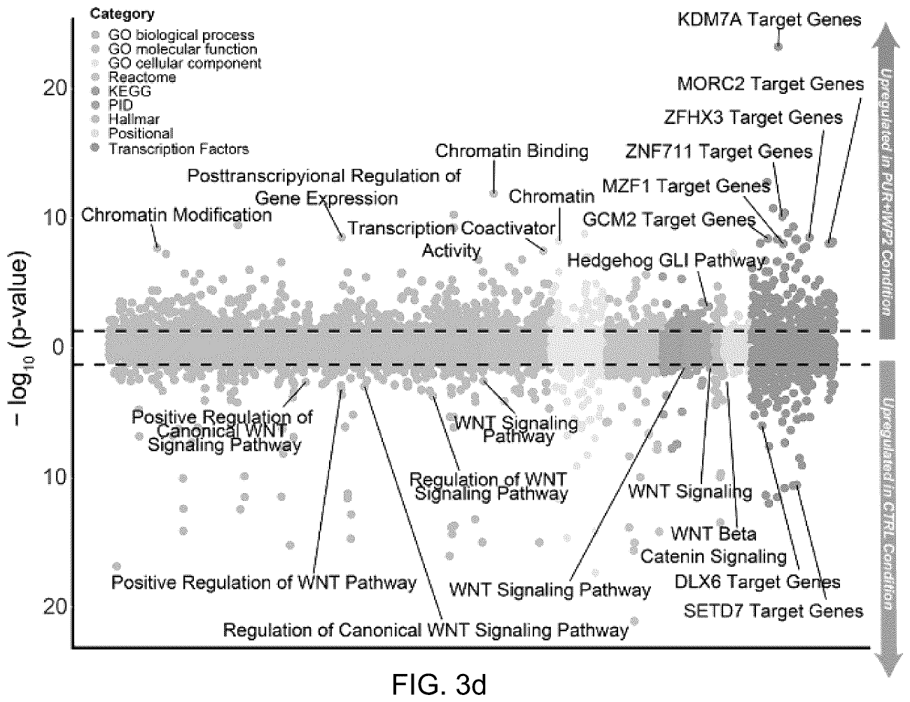

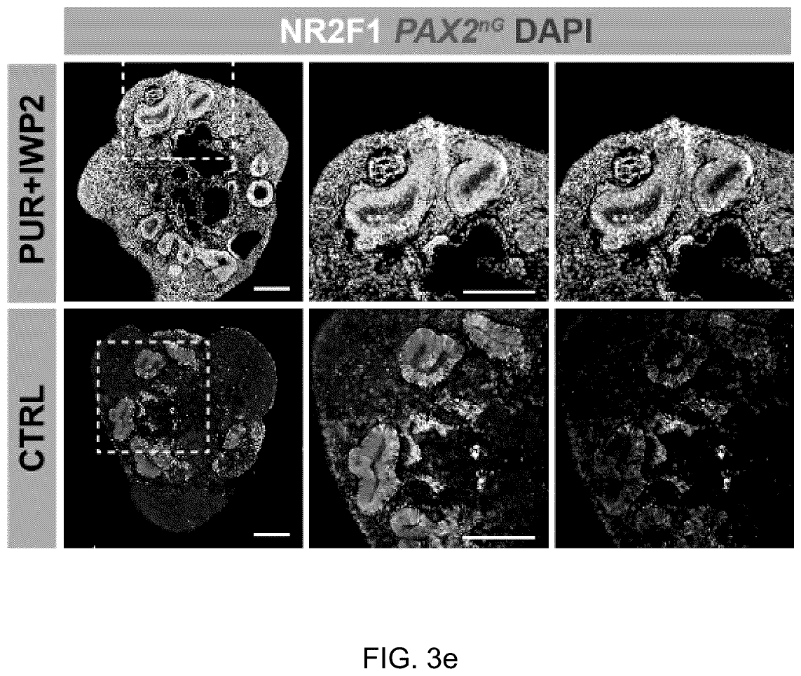

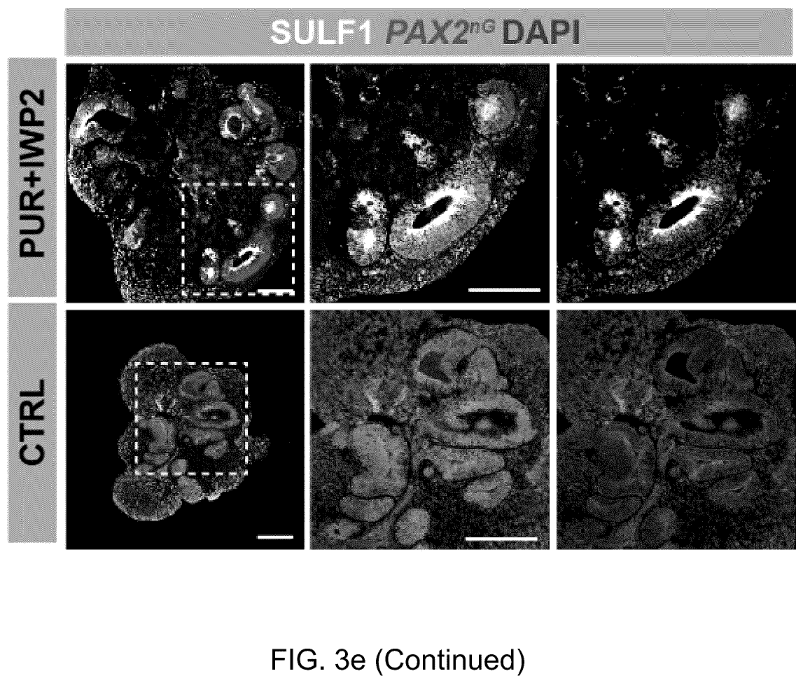

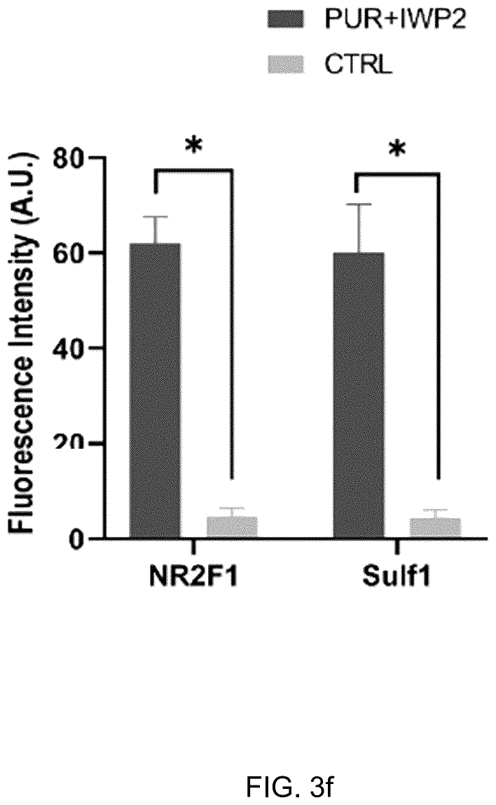

PAX2b⁺ otic progenitor cells can be derived from pluripotent stem cells. Suitably, the PAX2b cells are derived by day 10-13 from human pluripotent stem cells (day 0 being the start of differentiation medium, see, e.g., FIGS. 2a and 3a). In such instances, the PAX2b⁺ otic progenitor cells are cultured with the agonist of sonic hedgehog from about day 13 to about day 22. The cells are then subsequently cultured with the Wnt inhibitor from about day 18 to about day 22. By the subsequent addition of Wnt inhibitor to the agonist of SHH, the current methods are surprisingly able to differentiate the cells into cochlear hair cells which express GATA3, NR2F1 or INSM1. The expression of the given markers, e.g., GATA3, NR2F1 or INSM1, may be measured by any known technique, e.g., RNA sequencing, quantitative PCR (qPCR), enzyme-linked immunosorbent assay (ELISA), etc. PAX2b⁺ otic progenitor cells may be differentiated by: (a) culturing pluripotent stem cells in medium comprising FGF-2, BMP-4, and SB43 1542 for about 3 days on coated plates; (b) further culturing the cells of step (a) in medium comprising FGF-2, SB431542 and LDN193189 for about 4 days; (c) further culturing the cells of step (b) in medium comprising CHIR99021, LDN193189, and FGF-2 for about 4 days; further culturing the cells of step (c) in medium comprising CHIR99021 on coated plates for about 2 days, to generate PAX2b⁺ otic progenitor cells.

As used herein, “cochlear hair cells” refers to cells of the cochlea that transmit auditory sensation. Two types of cochlear hair cells are present in normal human cochlea, inner and outer hair cells. Outer hair cells possess the property of electromotility-synchronizing the length of the cell to incoming sound signal thereby providing mechanical amplification to the sound signal. This effect is termed the “cochlear amplifier” and is thought to improve frequency selectivity of the mammalian ear. Outer hair cells are characterized by expression of the motor protein PRESTIN which is encoded by the gene SLC26A5. Inner hair cells detect acoustic vibrations in the fluid of the cochlea and convert them into electrical signals that are relayed through the auditory nerve and into the brain. The cochlear hair cells derived herein can be detected using one or more of the markers selected from GATA3, INSM1, NR2F1, HES6, TMPRSS3, GNG8, or PRESTIN. In some embodiments, the cochlear hair cells express two or more markers selected from GATA3, INSM1, NR2F1, HES6, TMPRSS3, GNG8, or PRESTIN, in some alternatively embodiments, the cells express three or more markers or four or more markers selected from GATA3, INSM1, NR2F1, HES6, TMPRSS3, GNG8, or PRESTIN. In one embodiment, the cochlear hair cells express the four markers GATA3, NR2F1, INSM1 and PRESTIN, and in some embodiments, express further express one or more selected from HES6, TMPRSS3 and GNG8.

In contrast, “vestibular hair cells” are hair cells that transmit balance and gravity sensation. The vestibular organ comprises the semicircular canals and the utricle and saccule. During development, the cochlea is derived from the most ventral region of the otic vesicle, whereas the vestibular structure originates from a more dorsal otic region (and therefore express dorsal otic markers).

Therefore, inner ear cells of the cochlea are referred to as “ventralized” in comparison to inner ear cells of the vestibulary organ. Accordingly, in some embodiments, the invention disclosed herein represents novel methods of generating ventralized hair cells as opposed to previous methods which generated dorsal/vestibular hair cells.

The present methods apply aggregates of human pluripotent stem cells and found that modulations of Sonic Hedgehog and WNT signaling promote stem cell-derived otic progenitors to express ventral otic markers. Strikingly, some of these ventralized otic progenitors give rise to hair cells with short hair bundles comprised of stereocilia arrayed in a geometry reminiscent of cochlear hair cells. Moreover, these ventralized hair cells express multiple markers defining outer or inner hair cells in the cochlea. These results reveal that early morphogenic signals are sufficient for not only establishing cochlear gene expression, but also defining structural properties pertaining to the cochlear sensory epithelium.

Next, the inventors discovered that timed activation of the sonic hedgehog (SHH) pathway, with concomitant suppression of protein kinase A (PKA), bone morphogenic protein (BMP) and/or wingless (WNT, or Wnt) signalling may promote upregulation of ventral otic genes, leading to generation of cochlear hair cells in long-term culture. Briefly, otic progenitor cells are cultured in medium comprising an activator of sonic hedgehog signaling (activator of SHH) for at least 3 to 5 days. Next, the cells are cultured in medium comprising an activator of SHH and a Wnt inhibitor for a sufficient amount of time to differentiate the cells into human cochlear hair cells which express one or more of the cochlear hair cell markers of GATA3, INSM1, NR2F1 or PRESTIN. In some embodiments, the cochlear hair cells express PRESTIN. In some embodiments, the otic progenitor cells are cultured with the activator of SHH from about day 13 to about day 22, e.g., for about 10, 9, 8, 7, 6, 5, 4, 3, or 2 days. In some embodiments, the otic progenitor cells are cultured with the Wnt inhibitor from about day 18 to about day 22, e.g., for about 6, 5, 4, 3, 2, or 1 day(s), day 0 being the start of culturing aggregated stem cells in medium comprising a TGF-beta inhibitor, FGF2 and, optionally, BMP-4 (see, FIG. 2a). In the instant disclosure, it is to be understood that the culture DO begins at the start of the generation of otic progenitor cells from pluripotent stem cells (see FIG. 2A).

The inventors discovered that, when performing the disclosed methods, long-term culture (e.g., more than 50 days) produces cells expressing markers of mature cochlear hair cells, e.g., PRESTIN. Therefore, in some embodiments, subsequent to culturing the cells in media comprising activator of SHH and Wnt inhibitor, the cells are further cultured in organoid maturation medium (OMM). OMM comprises a 50:50 mixture of Advanced DMEM:F12 (Thermo Fisher, 12634028) and Neurobasal Medium (Thermo Fisher, 21103049) supplemented with 0.5× N2 Supplement (Thermo Fisher, 17502048), 0.5×B27 minus Vitamin A (Thermo Fisher, 12587010), 1× GlutaMAX (Thermo Fisher, 35050061), 0.1 mM β-Mercaptoethanol (Thermo Fisher, 21985023), and Normocin. In some embodiments, the cells are further cultured in OMM, e.g., for about 50, 60, 70, 80, 90, 100, 150, or more days. In some embodiments, the cells are cultured for greater than about 100 days, or greater than about 150 days. In some embodiments, long-term culture results in cells expressing one or more markers selected from PRESTIN, GATA3, INSM1, HES6, TMPRSS3 and GNG8, measured by expression of, e.g., mRNA or protein. Detection of the markers may be performed using any assay known in the art to detect expression of target molecules, e.g., immunofluorescence (IF), immunohistochemistry (IHC), fluorescent or luminescent reporters, quantitative polymerase chain reaction (qPCR), RNA sequencing (RNA-seq), single cell RNA seq (scRNA-seq), etc.

Exemplary activators of SHH include, but are not limited to the compounds SAG (3-Chloro-N-[trans-4-(methylamino)cyclohexyl]-N-[[3-(4-pyridinyl)phenyl]methyl]-benzo[b]thiophene-2-carboxamide dihydrochloride) and purmorphamine, an agonist of the protein smoothened, (9H-Purin-6-amine, 9-cyclohexyl-N-[4-(4-morpholinyl)phenyl]-2-(1-naphthalenyloxy)-. In some embodiments, the activator of SHH is purmorphamine. Suitable concentrations are known in the art. In some embodiments, the activator of SHH has a concentration of about 1 nM to about 1 mM in the culture medium, and suitably is purmorphamine.

Exemplary Wnt inhibitors include, but are not limited to IWP-2 (N-(6-Methyl-2-benzothiazolyl)-2-[(3,4,6,7-tetrahydro-4-oxo-3-phenylthieno[3,2-d]pyrimidin-2-yl)thio]-acetamide), Wnt-C49 (2-(4-(2-methylpyridin-4-yl)phenyl)-N-(4-(pyridin-3-yl)phenyl) acetamide), IWP L6 (2-[(4-oxo-3-phenyl-6,7-dihydrothieno[3,2-d]pyrimidin-2-yl) sulfanyl]-N-(5-phenylpyridin-2-yl) acetamide), and IWP 12 (2-[(3,6-dimethyl-4-oxo-6,7-dihydrothieno[3,2-d]pyrimidin-2-yl) sulfanyl]-N-(6-methyl-1,3-benzothiazol-2-yl) acetamide). In some embodiments, the Wnt inhibitor is IWP-2. Suitable concentrations can be determined by one skilled in the art. For example, in some embodiments, the Wnt inhibitor is in a concentration of about 1 nM to about 1 mM in the culture medium. The Wnt inhibitor may be IWP-2 and used at a concentration from about 1 nM to 1 mM.

Exemplary TGF-beta inhibitors include SB-431542, Galunisertib (LY2157299), LY2109761, SB525334, SB505124, GW788388, LY364947, RepSox (E-616452), TGFβRI-IN-3, R-268712, BIBF-0775, TP0427736 HCl, A-83-01, SD-208, and Vactosertib (TEW-7197). The TGF-beta inhibitor may be, preferably, SB-431542.

As described above, the cells cultured with both an activator of SHH and a Wnt inhibitor may be further cultured beyond the first 22 days, for example, the cells may be further cultured for at least 50 days, alternatively for greater than about 100 days, wherein the medium after about day 22-25 is without additional agonists or inhibitors after about 22-25 days. In some embodiments, the cells are washed before moving to medium without additional agonists or inhibitors. In some embodiment, the cells are cultured for greater than about 150 days in media without additional agonists or inhibitors.

The inventors discovered that culturing the cells from about day 50 to about day 100 in the presence of thyroxine, e.g., 250 ng/ml thyroxine, resulted in increased PRESTIN expression in the cells. Accordingly, the cells may be cultured in the absence of additional agonists or inhibitors after about 22-25 days, except for the presence of thyroxine. The cells after such extended culture express cochlear hair cell markers, for example, PRESTIN. Additionally, the cells express two or more of the markers selected from PRESTIN, GATA3, INSM1, HES6, TMPRSS3 and GNG8, alternatively three or more markers, alternatively four or more markers, alternatively five or more markers, alternatively all 6 markers associated with cochlear hair cells.

In some cases, the semi-solid composition of extracellular matrix proteins is a commercially available product such as Geltrex® basement membrane matrix. Geltrex® basement membrane matrix is suitable for use with human pluripotent stem cell applications using StemPro® hESC SFM or Essential 8™ media systems. In other cases, the semi-solid composition comprises two or more extra cellular matrix proteins such as, for example, laminin, entactin, vitronectin, fibronectin, a collagen, Matrigel™, or combinations thereof.

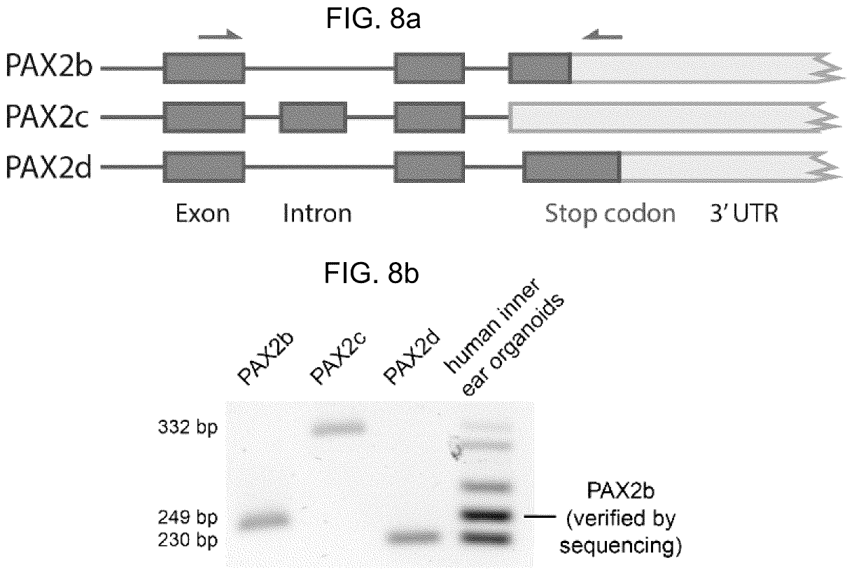

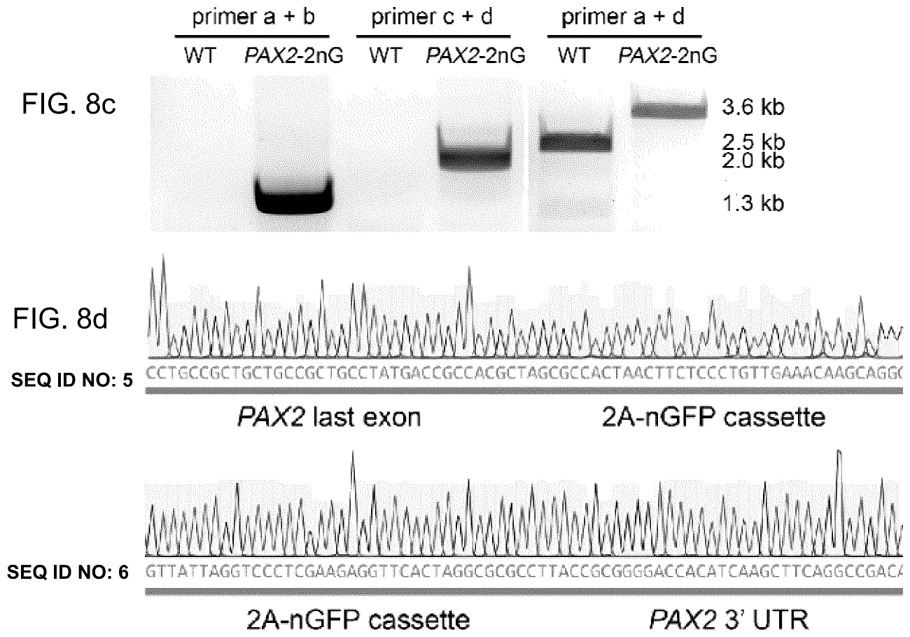

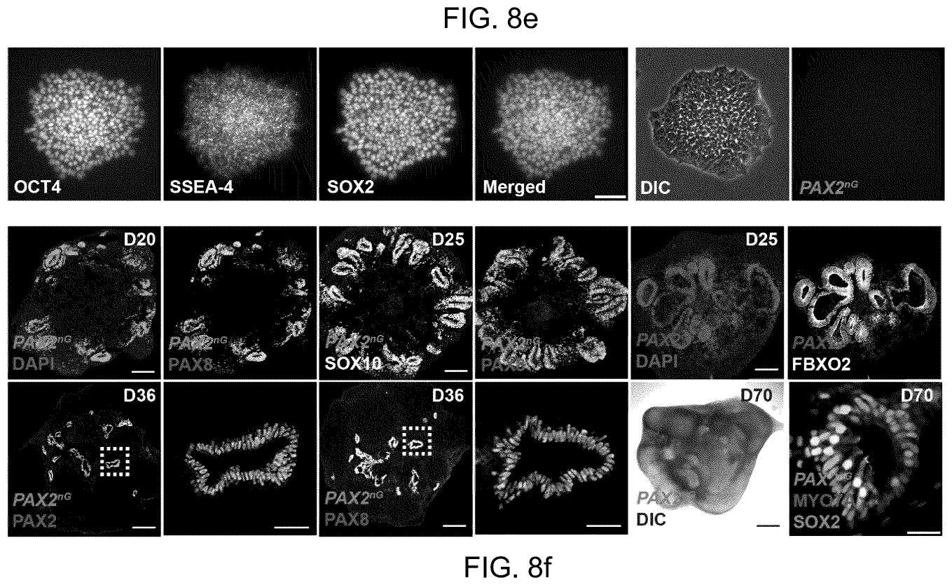

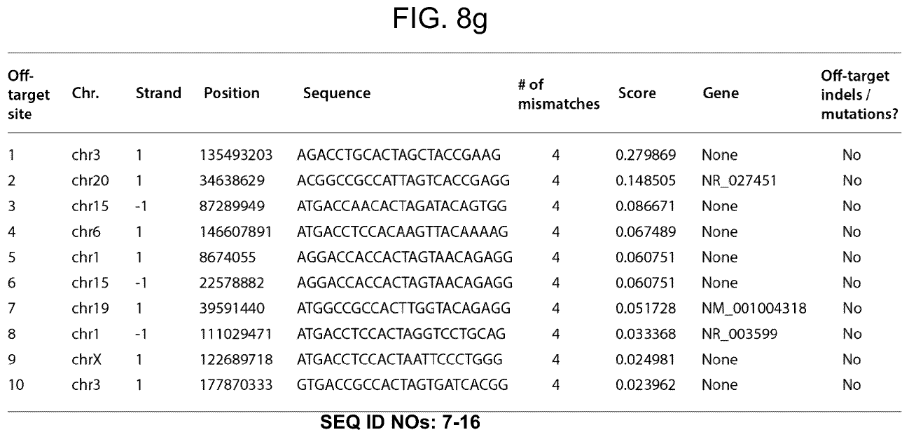

In some embodiments, the methods of the current disclosure begin with methods for generating “otic progenitor cells”. Otic progenitor cells are characterized by expression of PAX2 (e.g., PAX2 otic progenitor cells), and more specifically PAX2b in developing human inner ears (FIG. 8). Before cells are induced to become otic progenitor cells, the cells are, in some embodiments, aggregated. To form aggregates, a confluent culture of pluripotent stem cells can be chemically, enzymatically or mechanically dissociated from a surface, such as Matrigel® into clumps, aggregates, or single cells. In some embodiments, media used for aggregating cells of the current disclosure comprises Essential 8 Flex Medium (Thermo Fisher, A2858501) supplemented with 100 μg/ml Normocin (E8fn) on a suitable matrix, e.g., recombinant human Vitronectin-N, collagen, matrigel, etc. In some embodiments, the dissociated cells (as clumps, aggregates, or single cells) are plated onto a surface in a protein-free basal medium such as Dulbecco's Modified Eagle's Medium (DMEM)/F12, mTeSR™ (StemCell Technologies; Vancouver, British Columbia, Canada), and TeSR™. The full constituents and methods of use of TeSR™ are described in Ludwig et al. See, e.g., Ludwig T, et al., “Feeder-independent culture of human embryonic stem cells,” Nat. Methods 3:637-646 (2006); and Ludwig T, et al., “Derivation of human embryonic stem cells in defined conditions,” Nat. Biotechnol. 24:185-187 (2006), each of which is incorporated herein by reference as if set forth in its entirety. Other DMEM formulations suitable for use herein include, e.g., X-Vivo (BioWhittaker, Walkersville, Md.) and StemPro® (Invitrogen; Carlsbad, Calif.).

As used herein, the term “pluripotent cell” means a cell capable of differentiating into cells of all three germ layers, i.e., ectoderm, mesoderm, and endoderm. Examples of pluripotent cells include embryonic stem cells and induced pluripotent stem (iPS) cells. As used herein, “iPS cells” refer to cells that are substantially genetically identical to their respective differentiated somatic cell of origin and display characteristics similar to higher potency cells, such as ES cells, as described herein. The cells can be obtained by reprogramming non-pluripotent (e.g., multipotent or somatic) cells. Pluripotent stem cells (PSCs) suitable for the differentiation methods disclosed herein include, but are not limited to, human embryonic stem cells (hESCs), human induced pluripotent stem cells (hiPSCs), non-human primate embryonic stem cells (nhpESCs), non-human primate induced pluripotent stem cells (nhpiPSCs).

Subject-specific somatic cells for reprogramming into iPS cells can be obtained or isolated from a target tissue of interest by biopsy or other tissue sampling methods. In some cases, subject-specific cells are manipulated in vitro prior to use. For example, subject-specific cells can be expanded, differentiated, genetically modified, contacted to polypeptides, nucleic acids, or other factors, cryo-preserved, or otherwise modified.

Defined media and substrate conditions for culturing pluripotent stem cells, as used in the methods described herein, are well known in the art. In some exemplary embodiments, pluripotent stem cells to be differentiated according to the methods disclosed herein are cultured in mTESR®—1 medium (StemCell Technologies, Inc., Vancouver, Calif.), or Essential 8® medium (Life Technologies, Inc.) on a Corning® Synthemax® surface or, in some cases, a Matrigel® substrate (BD Biosciences, NJ) according to the manufacturer's protocol.

In some embodiments, aggregates of pluripotent stem cells are optionally cultured in the presence of a Rho kinase (ROCK) inhibitor. Kinase inhibitors, such as ROCK inhibitors, are known to protect single cells and small aggregates of cells. See, e.g., U.S. Patent Application Publication No. 2008/0171385, incorporated herein by reference in its entirety; and Watanabe K, et al., “A ROCK inhibitor permits survival of dissociated human embryonic stem cells,” Nat. Biotechnol. 25:681-686 (2007), incorporated herein by reference. ROCK inhibitors are shown below to significantly increase pluripotent cell survival on chemically defined surfaces. ROCK inhibitors suitable for use herein include, but are not limited to, (S)-(+)-2-methyl-1-[(4-methyl-5-isoquinolinyl) sulfonyl]homopiperazine dihydrochloride (informal name: H-1152), 1-(5-isoquinolinesulfonyl) piperazine hydrochloride (informal name: HA-100), 1-(5-isoquinolinesulfonyl)-2-methylpiperazine (informal name: H-7), 1-(5-isoquinolinesulfonyl)-3-methylpiperazine (informal name: iso H-7), N-2-(methylamino) ethyl-5-isoquinoline-sulfonamide dihydrochloride (informal name: H-8), N-(2-aminoethyl)-5-isoquinolinesulphonamide name: dihydrochloride (informal H-9), N-[2-p-bromo-cinnamylamino) ethyl]-5-isoquinolinesulfonamide dihydrochloride (informal name: H-89), N-(2-guanidinoethyl)-5-isoquinolinesulfonamide hydrochloride (informal name: HA-1004), 1-(5-isoquinolinesulfonyl) homopiperazine dihydrochloride (informal name: HA-1077), (S)-(+)-2-Methyl-4-glycyl-1-(4-methylisoquinolinyl-5-sulfonyl) homopiperazine dihydrochloride (informal name: glycyl H-1152) and (+)-(R)-trans-4-(1-aminoethyl)-N-(4-pyridyl)cyclohexanecarboxamide dihydrochloride (informal name: Y-27632). The kinase inhibitor can be provided at a concentration sufficiently high that the cells survive and remain attached to the surface. An inhibitor concentration between about 3 μM to about 10 UM may be suitable for the disclosed methods. At lower concentrations, or when no ROCK inhibitor is provided, undifferentiated cells typically detach, while differentiated cells remain attached to the defined surface.

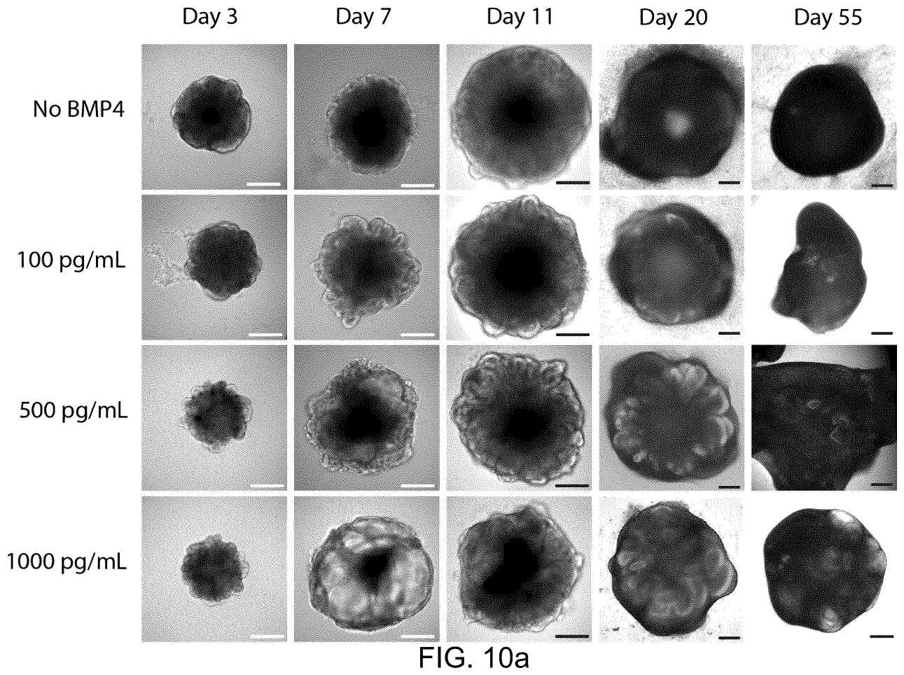

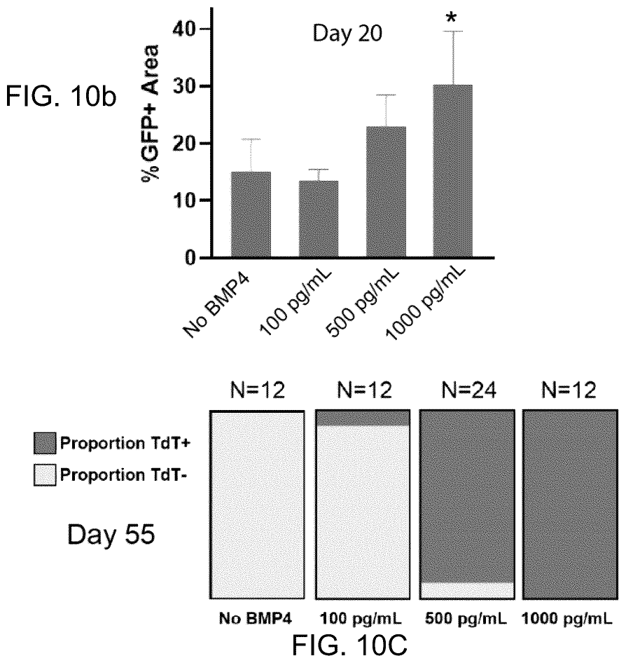

To induce formation of otic progenitor cells, aggregated pluripotent stem cells are cultured in media comprising an inhibitor of transforming growth factor β (TGF-β) signaling, e.g., SB-431542, bone morphogenic protein (BMP-4) and low concentration of fibroblast growth factor 2 (FGF-2) for about 3 days. The inventors discovered that culturing the aggregated stem cells with BMP-4 at this stage of the differentiation protocol increases the proportion of POU4F3 cells at D55 after the start of culturing the aggregates (FIG. 10a). Further, the concentration of BMP-4 affects the proportion of POU4F3 cells (FIGS. 10b, 10c) and may be greater than about 100 μg/ml, greater than about 200 μg/ml, greater than about 300 μg/ml, greater than about 400 μg/ml, greater than about 500 μg/ml, greater than about 600 μg/ml, greater than about 700 μg/ml, greater than about 800 μg/ml, greater than about 900 μg/ml. The concentration of BMP-4 may be between about 100 μg/ml and about 500 μg/ml, e.g., about 200, 300, 400, or 500 μg/ml, or between about 500 μg/ml and about 1000 μg/ml, e.g., about 500, 600, 700, 800, 900 or 1000 μg/ml. Then, the cells are cultured in media comprising an inhibitor of bone morphogenic protein 4 (BMP-4) signaling, e.g., LDN 193189, and high concentration of FGF-2 for about 4 days, then the cells are cultured in media comprising high concentration FGF-2, BMP-4 signaling inhibitor, and an inhibitor of glycogen synthase kinase 3 (GSK-3), e.g., CHIR99021, for about 4 days. See FIG. 2a.

In some embodiments, otic progenitor cells are derived from pluripotent stem cells by the method of: (i) culturing the pluripotent stem cells in medium comprising FGF-2 and a TGF-beta inhibitor, e.g., SB-431542, from Day 0 to Day 3 on coated plates; (ii) culturing the cells of step (i) in medium comprising FGF-2, TGF-beta inhibitor and BMP-4 inhibitor, e.g., LDN 193189 for Days 3-Day 7; (iii) culturing the cells of step (ii) in medium comprising GSK-3 Inhibitor, e.g., CHIR99021, BMP-4 inhibitor from Day 7-Day 11; and (iv) culturing the cells of step (iii) in medium comprising GSK-3 inhibitor on coated plates from Day 11-18 to produce PAX⁺ otic progenitor cells.

### Cellular Compositions

In another aspect of the current disclosure, cellular compositions are provided. In some embodiments, the compositions comprise human cochlear hair cells or organoids comprising human cochlear hair cells generated by the methods described herein. For example, the human cochlear hair cells is generated by (a) culturing PAX2 otic progenitor cells derived from human pluripotent stem cells in medium comprising an activator of sonic hedgehog for at least 3 to 5 days; and (b) subsequently culturing the cells of step (a) in medium comprising an activator of sonic hedgehog and a Wnt inhibitor for a sufficient amount of time to differentiate the cells into human cochlear hair cells which express one or more of the otic markers of GATA3 or NR2F1. The cochlear hair cells may express PRESTIN. The cells of step (b) may be further cultured in media without activator of SSH or WNT inhibitor for at least 50 days, alternatively at least 100 days, alternatively at least 150 days and the cochlear hair cells express two or more markers selected from PRESTIN, NR2F1, GATA3, INSM1, HES6, TMPRSS3 and GNG8. The cochlear hair cells may express PRESTIN, NR2F1, GATA3 and INSM1. The cells may further express one or more of HES6, TMPRSS3 or GNG8. These cochlear hair cells can transmit auditory sensation, see, for example, FIGS. 6d-6h.

The otic progenitor cells may be derived from pluripotent stem cells by the method of: (i) culturing the pluripotent stem cells in medium comprising FGF-2 and a TGF-beta inhibitor, e.g., SB-431542, from Day 0 to Day 3 on coated plates; (ii) culturing the cells of step (i) in medium comprising FGF-2, TGF-beta inhibitor and BMP-4 inhibitor, e.g., LDN 193189 for Days 3-Day 7; (iii) culturing the cells of step (ii) in medium comprising GSK-3 Inhibitor, e.g., CHIR99021, BMP-4 inhibitor from Day 7-Day 11; and (iv) culturing the cells of step (iii) in medium comprising GSK-3 inhibitor on coated plates from Day 11-18 to produce PAX⁺ otic progenitor cells.

### Kits, Platforms, and Systems

In an aspect of the current disclosure, kits, systems, and platforms are provided. The kits, systems, or platforms may comprise one or more of: an activator of sonic hedgehog, a Wnt inhibitor, FGF-2, a TGF-beta inhibitor, a BMP-4 inhibitor, a GSK-3 inhibitor, thyroxine, and induced pluripotent stem cells or embryonic stem cells. The kits, systems, or platforms may also comprise a solid support, laminin, entactin, vitronectin, fibronectin, a collagen, Matrigel™, or combinations thereof.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1—Engineering High-Fidelity Cochlear Organoids from Human Pluripotent Stem Cells

The human inner ear is one of the most elaborate organs in the body with a snail-shaped cochlea and three orthogonal semicircular canals comprising the vestibular end organ. In the former resides two distinctive types of mechanosensitive hair cells (HCs) arranged in orderly rows. Inner ear morphogenesis is orchestrated by interwoven signaling events during fetal development¹⁻³. Inner ear development during embryogenesis succeeds without errors greater than 99% of the time. Indeed, while almost 10% of adults have moderate-to-profound hearing loss, the incidence at birth is less than 0.2%, meaning that the vast majority of sensorineural hearing loss results from the post-natal death or dysfunction of cells within a developmentally pristine inner ear 4.

To recapitulate the intricate process of human inner ear development in vitro, the inventors previously established defined 3D culture systems to generate inner ear sensory epithelia from aggregates of mouse or human pluripotent stem cells⁵⁻⁸. These so-called “inner ear organoids” harbour a layer of supporting cells and functional hair cells that are innervated by sensory-like neurons. However, these organoids consistently generate hair cells with structural, biochemical and functional properties comparable to those of native vestibular hair cells but fail to produce any cochlear cell types. In order to more properly model human inner ear development, the inventors aimed to establish a new organoid system that contains outer and inner hair cells, the two mechanosensitive hair cells in the cochlea that are essential for proper detection of the auditory stimuli.

### Results

A Multiplex Reporter hESC Line Provides Enhanced Culture Optimization

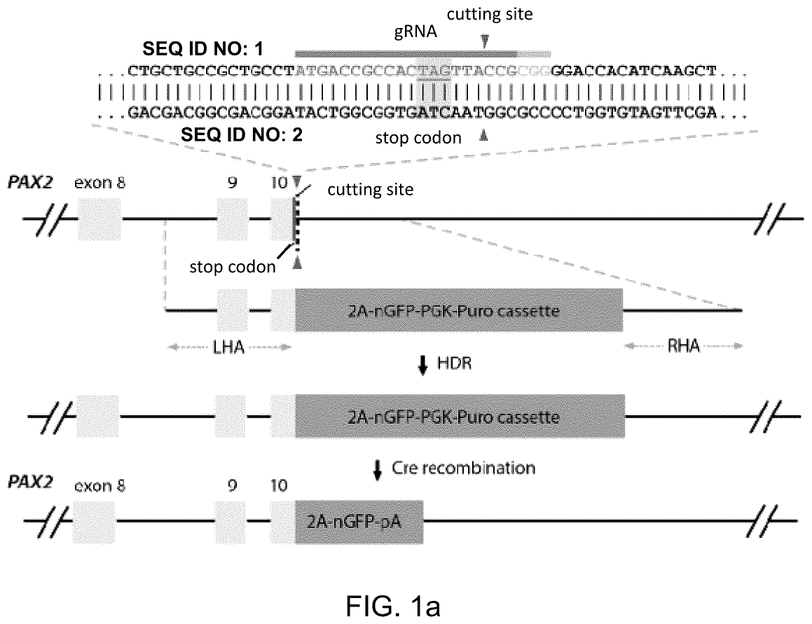

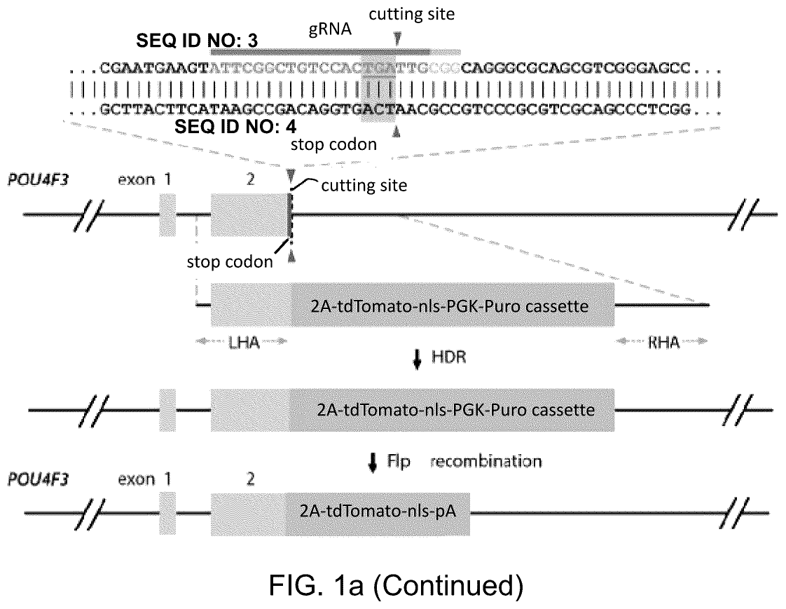

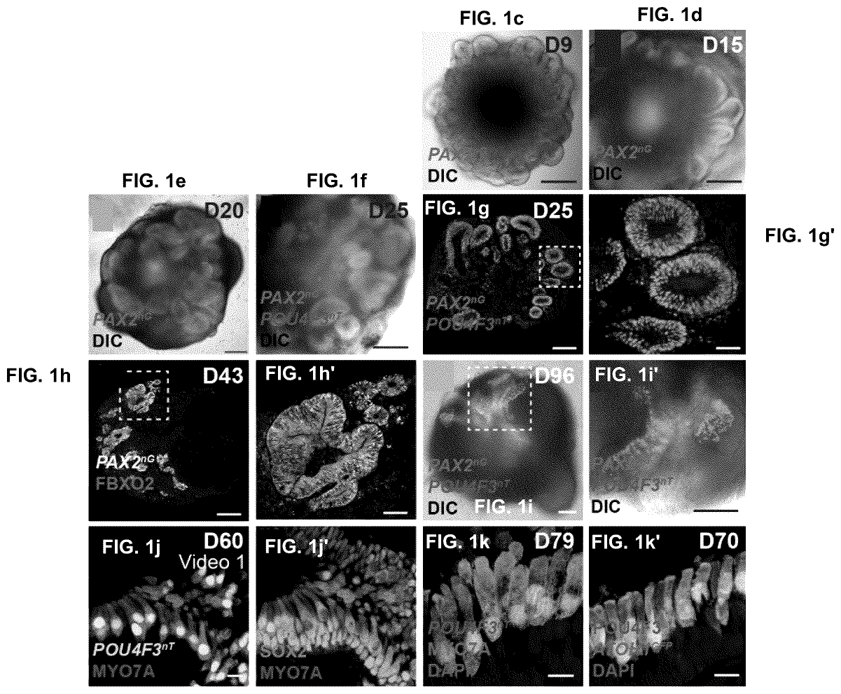

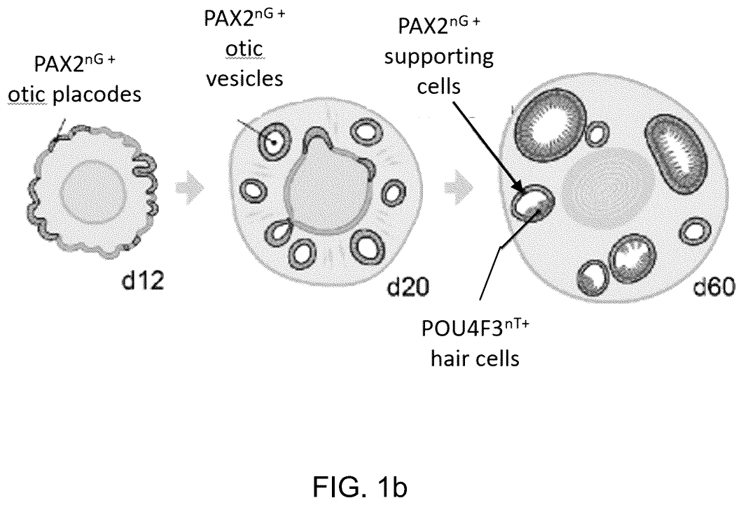

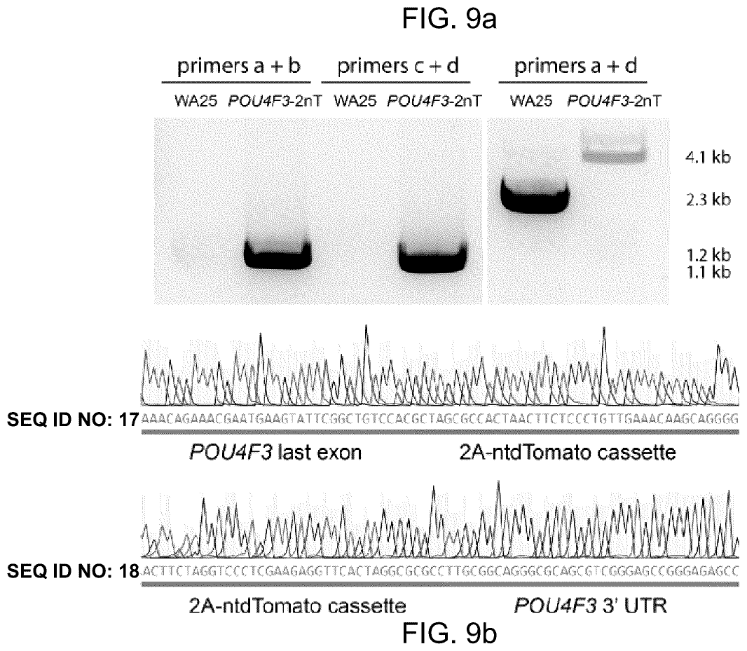

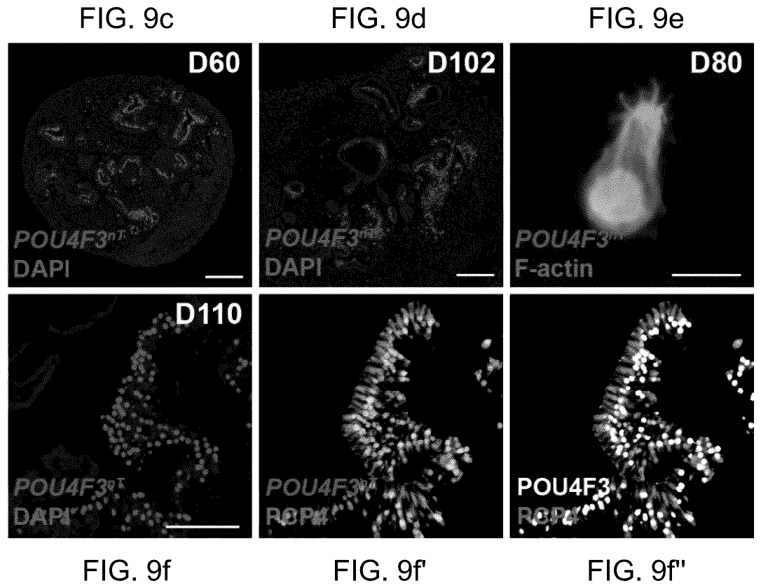

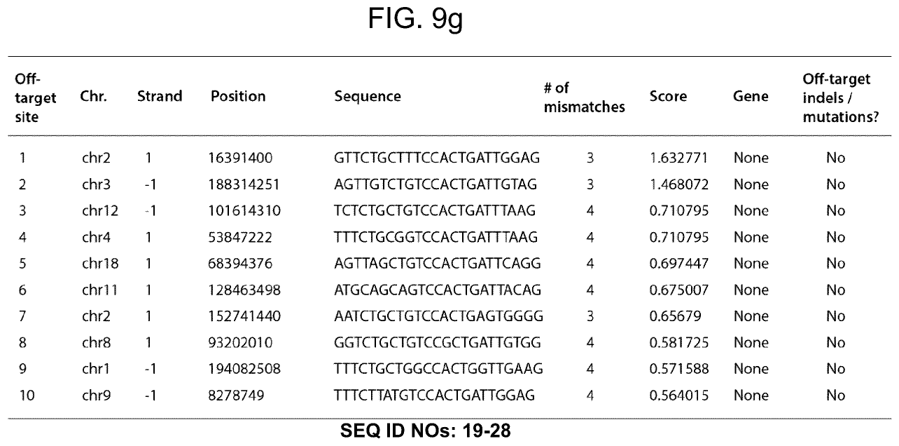

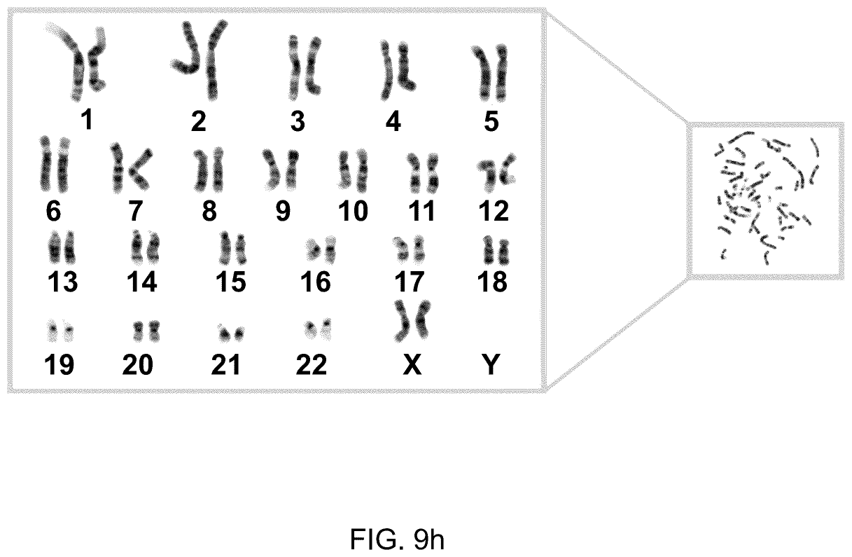

To monitor the derivation of otic progenitors and hair cells in culture and improve the efficiency of the inventors' previously published protocol^{5,8}, the inventors generated a PAX2-2A-nGFP/POU4F3-2A-ntdTomato reporter human embryonic stem cell (hESC) line using the CRISPR/Cas9 genome engineering technology. PAX2 is an early marker of otic progenitors in vivo, while POU4F3 expression is highly specific to hair cells and provides a late-stage readout of culture efficiency. The inventors first confirmed that PAX2b is the most abundantly expressed PAX2 isoform in human inner ear organoid tissues (FIG. 8a-b). Using a high-fidelity Cas9⁹ and a sgRNA targeting the stop codon of this isoform, the inventors knocked in a 2A-nGFP cassette immediately downstream of the endogenous PAX2 coding sequence. A 2A-ntdTomato cassette was similarly knocked in to the POU4F3 locus downstream of the POU4F3 stop codon using the established PAX2-2A-nGFP hESCs as a parental cell line (FIG. 1a-b). The resulting multiplex reporter cell line labels the PAX2+ otic progenitors with nuclear GFP starting from around organoid culture day 12 (D12), and the POU4F3+ hair cells with nuclear tdTomato starting from around D35 (FIG. 1c-m; FIG. 8-9). Using the hESC reporter line the inventors had established, the inventors systematically changed basal media, timings and durations of small molecule treatment to optimize otic induction (FIG. 2). Moreover, while all otic lineage differentiation requires BMP signalling to establish the non-neural ectoderm, the inventors found that the inventors' cochlear organoid cultures were especially sensitive to this parameter for efficient otic induction and subsequent hair cell generation (FIG. 10). Following optimization, the number of hair cell-producing aggregates more than doubled vs. the inventors' previous protocol 8.10, and the number of hair cells per aggregate increased 20-fold.

### Ventralization of Inner Ear Organoids by Sequential Modulations of the SHH and WNT Pathways

During inner ear development, the cochlear structure is derived from the most ventral region of the otic vesicle, whereas the vestibular structures originate from a more dorsal otic region¹. The inventors' culture platform relies in part on self-guided differentiation and patterning, and similar to previously reported inner ear organoid protocols, the inventors' optimized basal control (CTRL) culture system consistently generates hair cells of a vestibular phenotype. The inventors hypothesized that, as in embryonic development, signals exogenous to the otocyst may be necessary for cochlear induction. Sonic Hedgehog (SHH), a signaling molecule secreted from the floor plate of the neural tube and underlying notochord, is essential for patterning of the ventral otic vesicle. SHH is both necessary and sufficient to ventralize the otocyst and derive cochlear structures in mice and chicks^{11,12}. A more recent study substantiated these previous reports and demonstrated that SHH pathway activation leads to inhibition of cAMP-dependent protein kinase A (PKA), thereby reducing the proteolytic processing of the SHH downstream target GLI3 and regulating the expression of ventral-associated genes¹². In contrast, WNT and BMP signaling pathways have been shown to play a role in induction of dorsal gene expression during inner ear development^{13,14}.

Based on these previous mouse genetics studies, the inventors hypothesized that timed activation of the SHH pathway, with concomitant suppression of PKA, BMP and/or WNT signaling may promote upregulation of ventral otic genes, leading to generation of cochlear hair cells in long-term culture (FIG. 3a). To examine this hypothesis, the inventors cultured hESC-derived aggregates in the presence of the small-molecule SHH agonist purmorphamine (PUR) alone or in combinations with inhibitors of BMP (LDN), WNT (IWP2), and PKA (H89) (FIG. 3a). For brevity, the permutations presented here are not exhaustive, and include results from SHH activation alone (PUR) or in combination with WNT inhibition (PUR+IWP2).

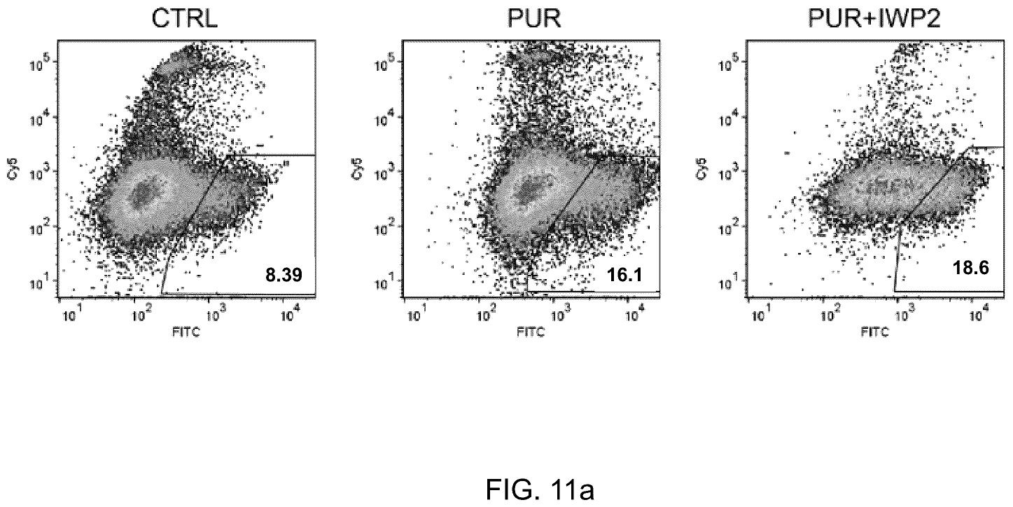

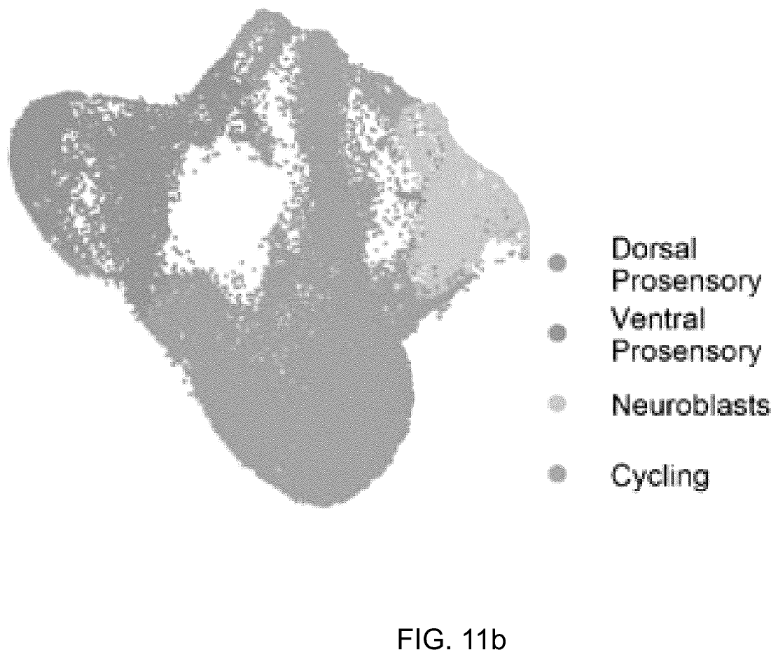

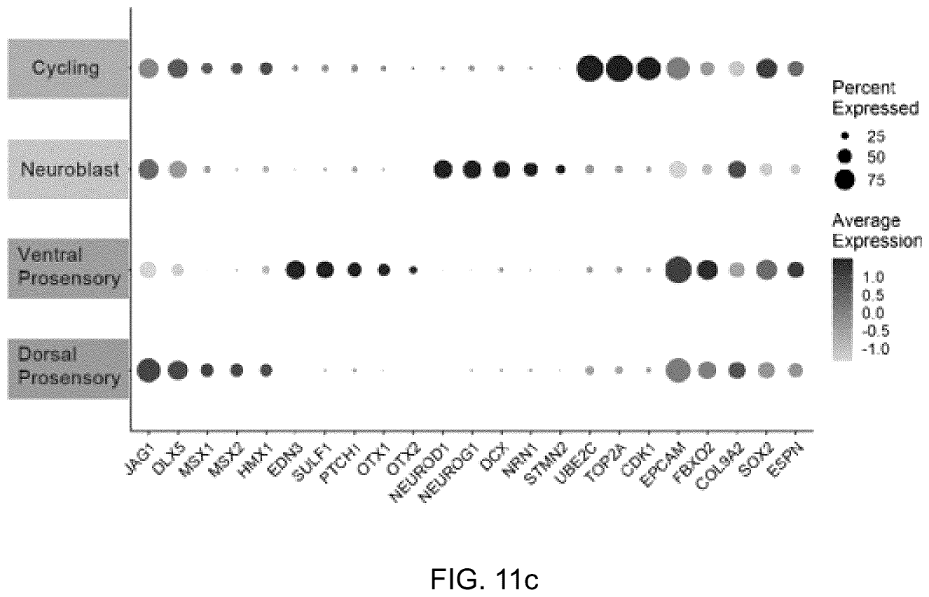

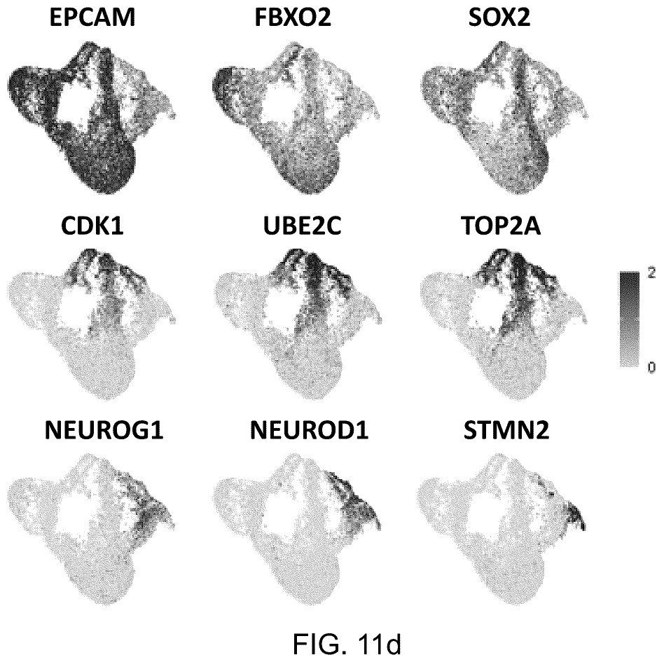

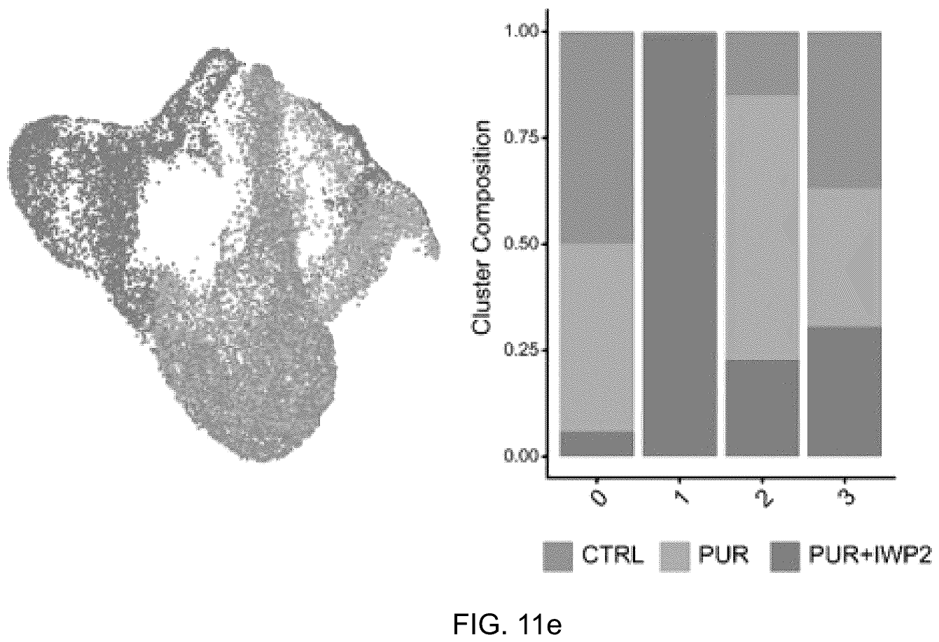

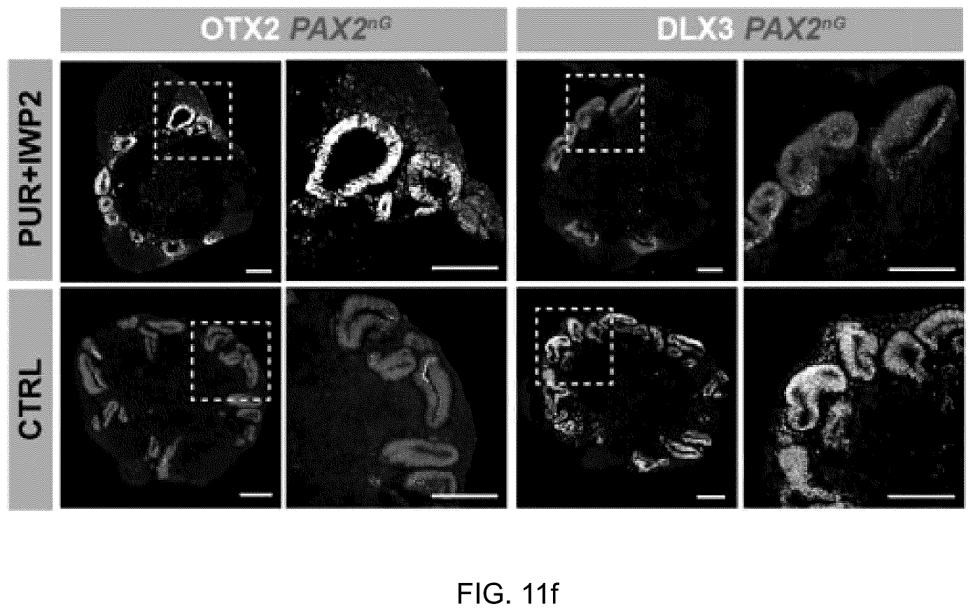

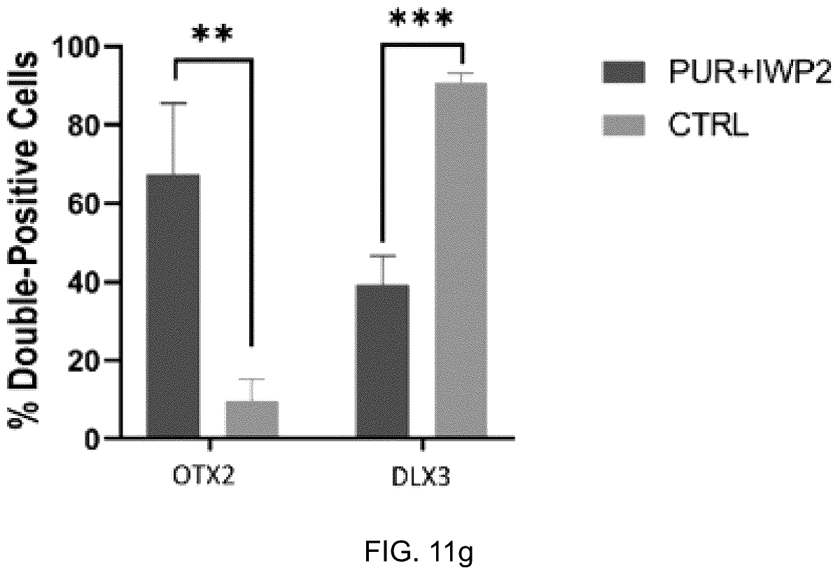

The inventors performed high-throughput single-cell RNA-sequencing (scRNA-seq) analysis on D20 PAX2-nGFP+ sorted cells from aggregates generated under CTRL, PUR or PUR+IWP2 conditions. Each condition was analysed separately, one batch per condition. A total of 37,073 cells were collected, and the cells from each batch were merged and clustered together. Unbiased clustering, as implemented in Seurat v3.2, revealed that EPCAM/FBX02+ otic progenitors from the PUR+IWP2 condition formed a distinct cluster, whereas PUR and CTRL otic progenitors clustered together (FIG. 3b; FIG. 11). Otic progenitors from all three conditions were isolated, and subsequent differential expression analysis among conditions revealed that dorsal otic markers such as DLX5, MSX1, GPR166, and ACSL4, were largely confined to PUR and CTRL treated cells, whereas ventral otic markers, including OTX1/2, NR2F1/2, EDN3, and RSPO3 were concentrated in the PUR+IWP2 condition. Moreover, genes involved in the SHH-pathway, such as SULF1, LRP2, GAS1, and PTCH1 were highly expressed in the PUR+IWP2-treated cells, but their expression was attenuated in CTRL and PUR-treated progenitors (FIG. 3c). Consistent with this, Gene Set Enrichment Analysis (GSEA)¹⁵ revealed enrichment of gene sets associated with the Hedgehog pathway and downregulation of gene sets associated with the canonical WNT pathway in PUR+IWP2 treated otic progenitors when compared to untreated CTRL otic progenitors. Additionally, analysis of PUR+IWP2 treated otic progenitors revealed multiple enriched gene sets for posttranscriptional regulation of gene expression, chromatin modifications, and gene sets composed of the targets of known hereditary deafness genes coding for transcription factors such as ZNF711, MORC2, GCM2 and BARHLI (FIG. 3d).

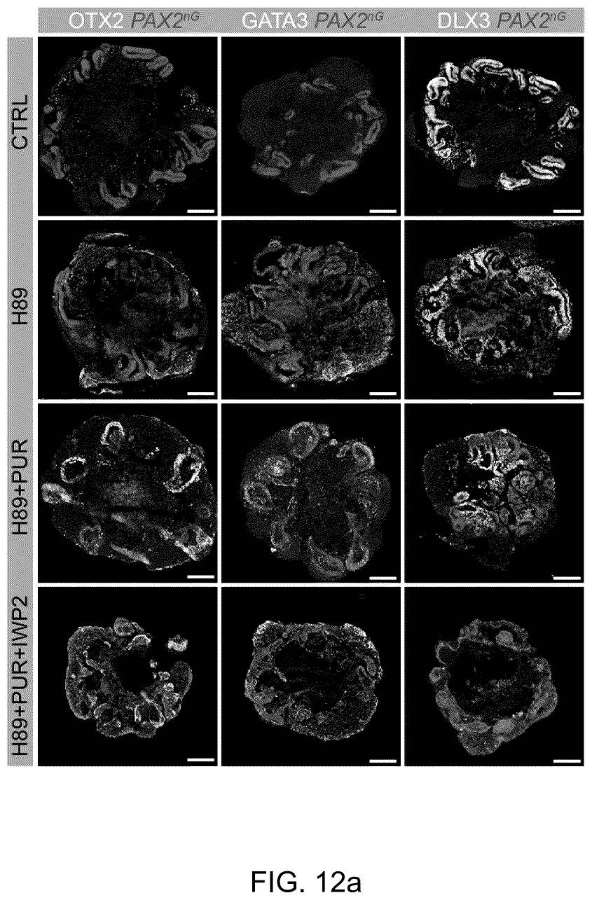

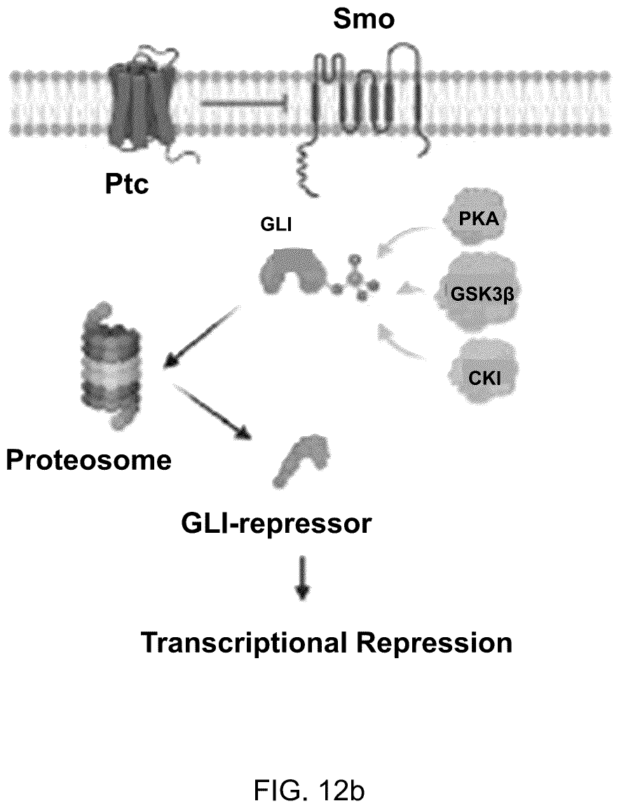

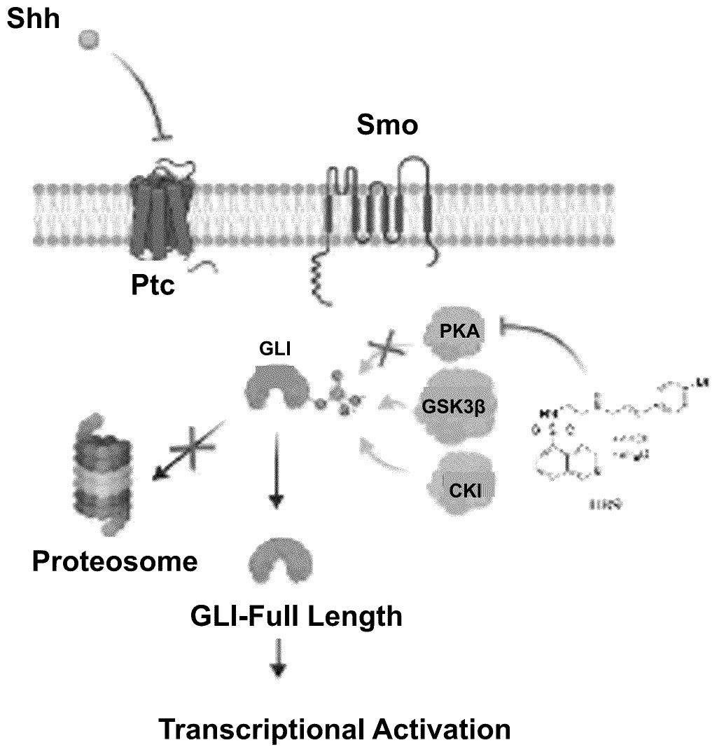

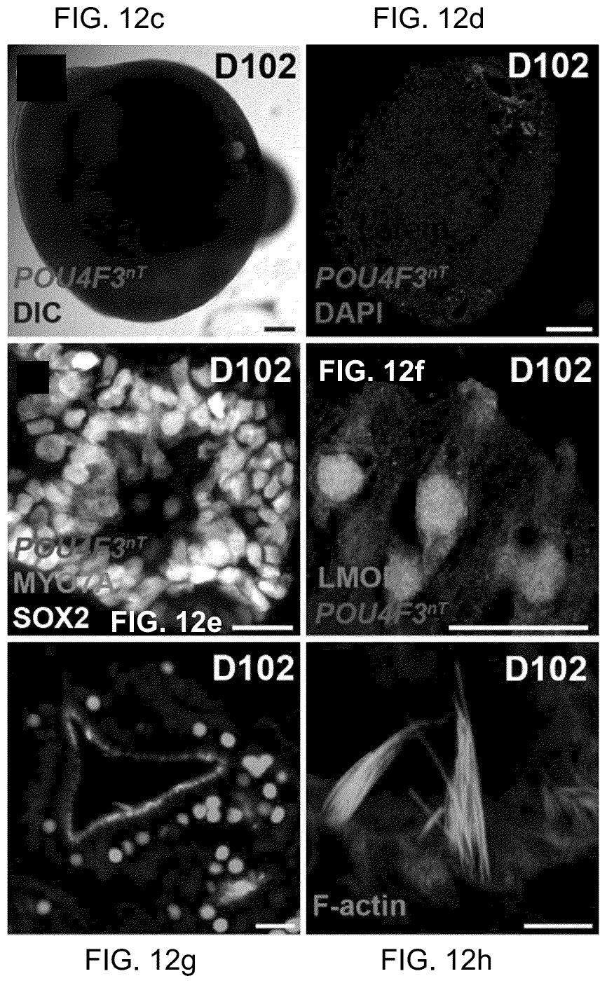

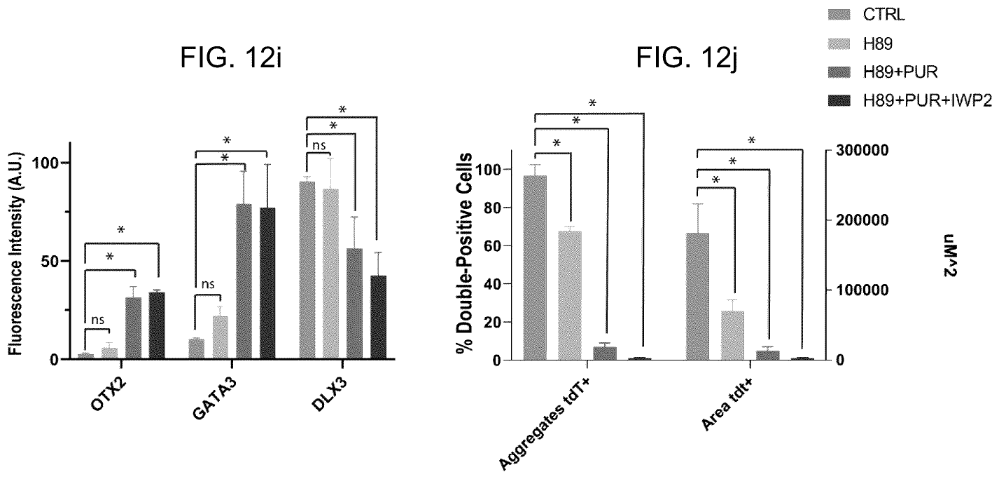

The inventors substantiated the scRNA-seq data with immunofluorescence for NR2F1, SULF1, and OTX2 using D25 organoids (FIG. 3e; FIG. 11). The inventors concluded that a more efficient ventralization of hESC-derived otic progenitors after sequential treatment with PUR and IWP2 is due to, at least partially, modulation of SHH down-stream gene targets. The inventors examined effects of the small molecule H89, a cell permeable PKA inhibitor, alone or in combination with PUR and IWP2, but none of these treatments were efficacious in deriving hair cells (FIG. 12).

### Otic Progenitors in Ventralized Inner Ear Organoids Give Rise to Cochlear Hair Cells

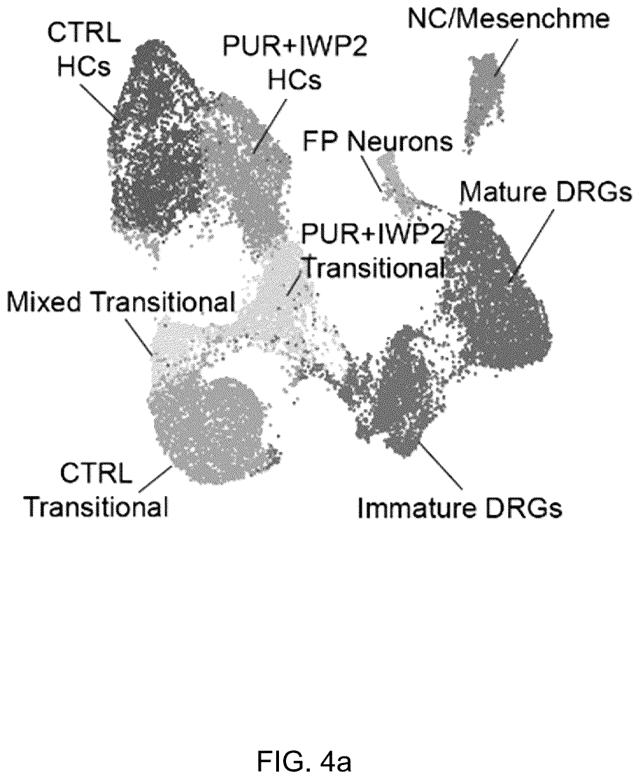

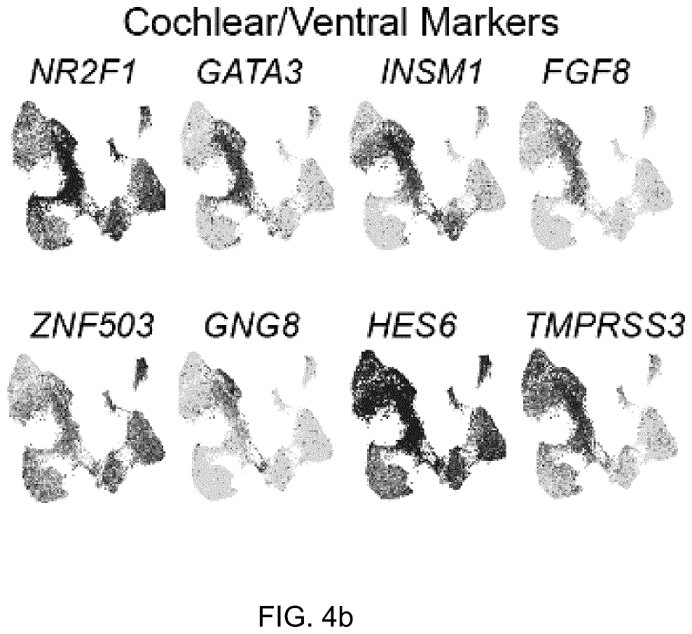

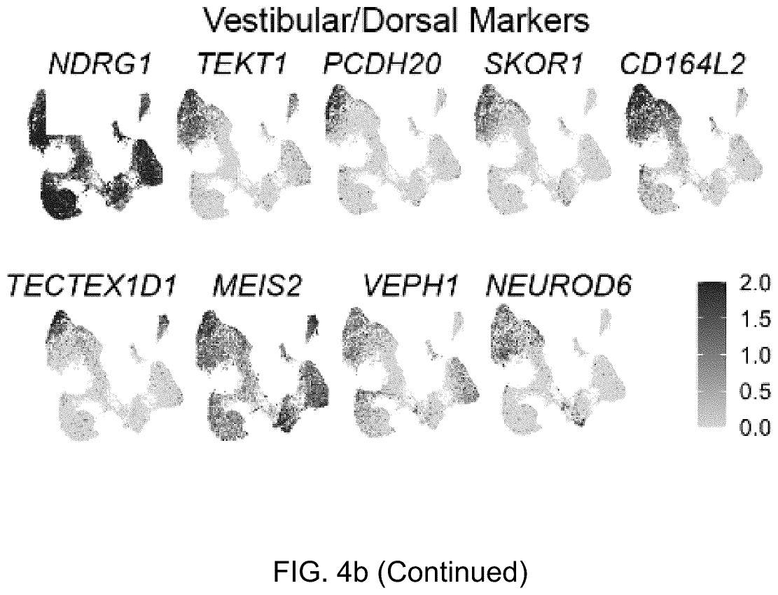

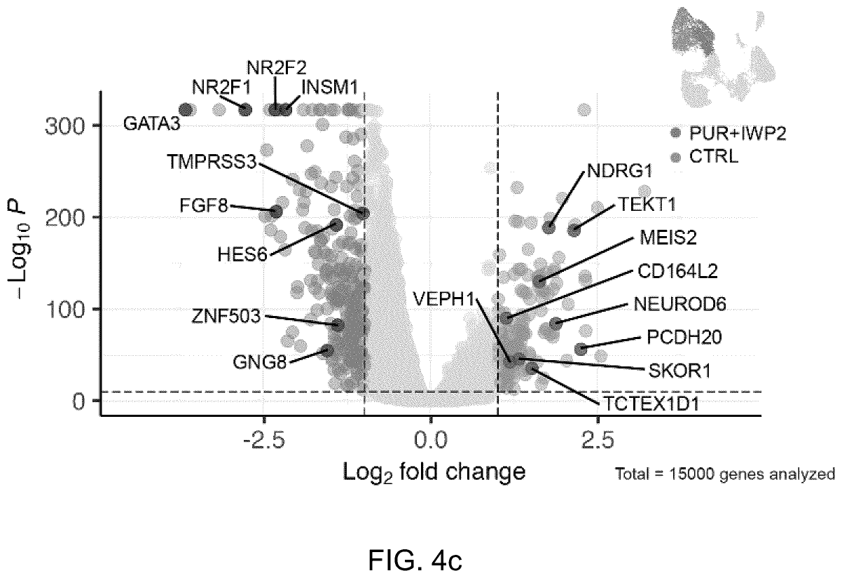

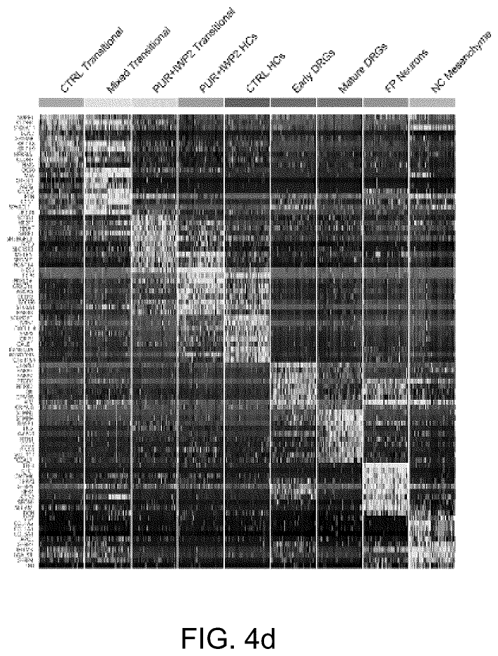

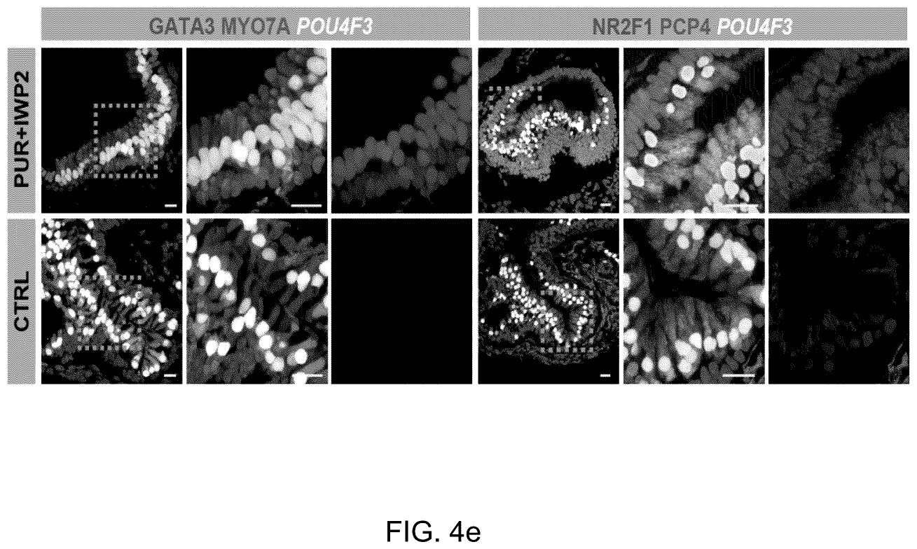

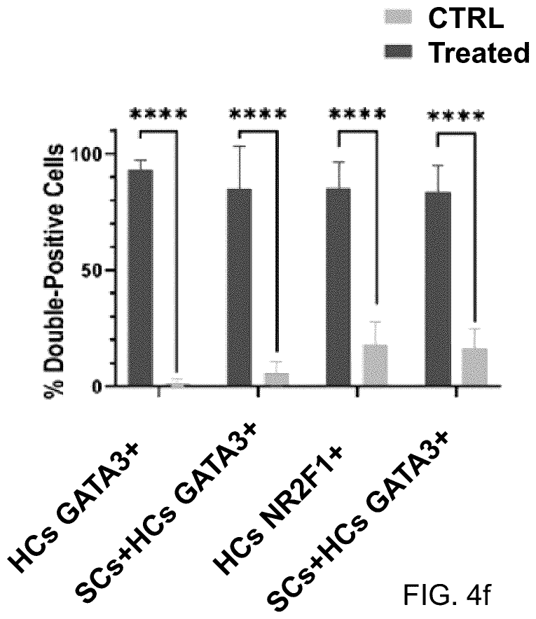

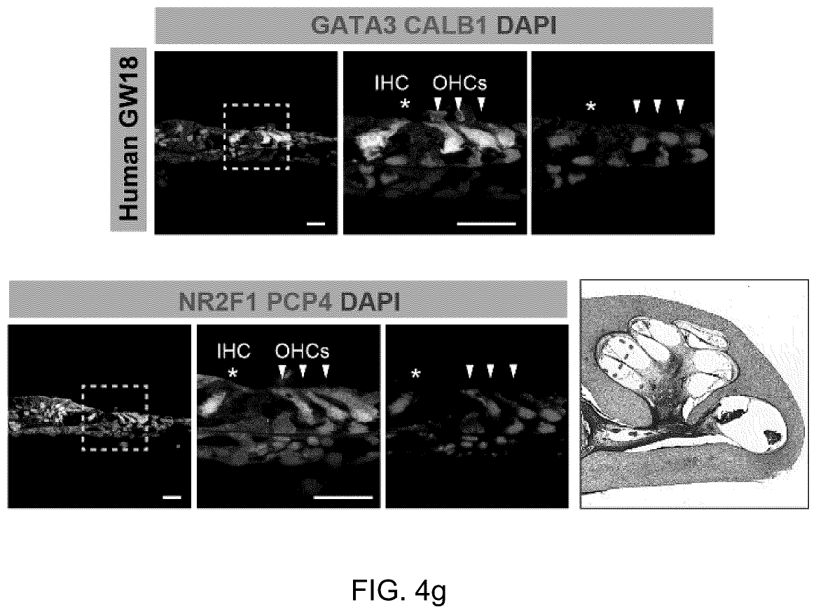

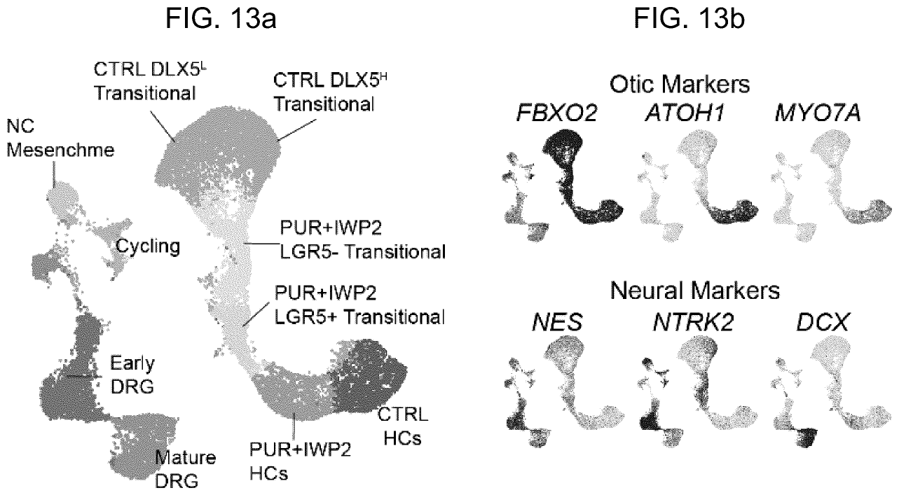

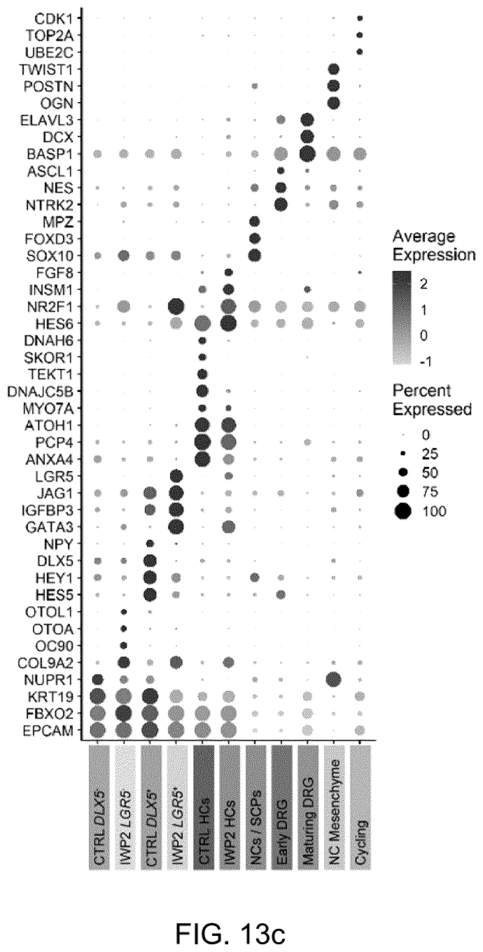

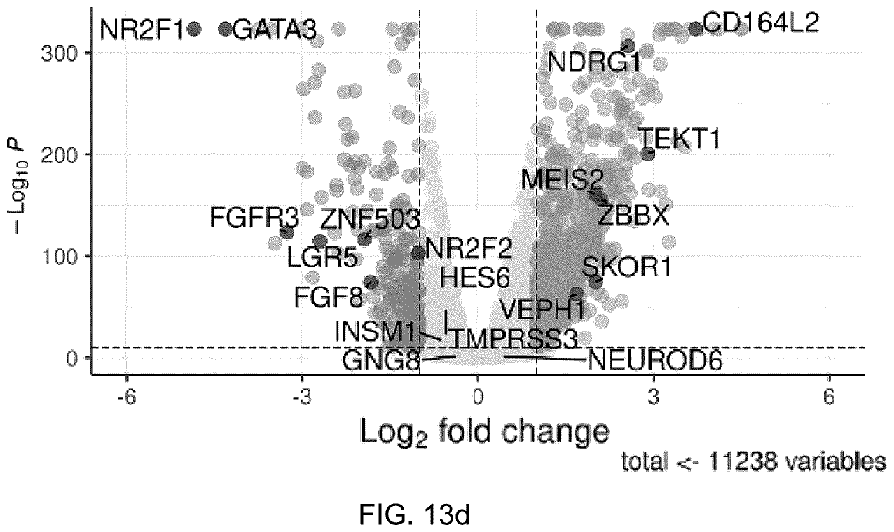

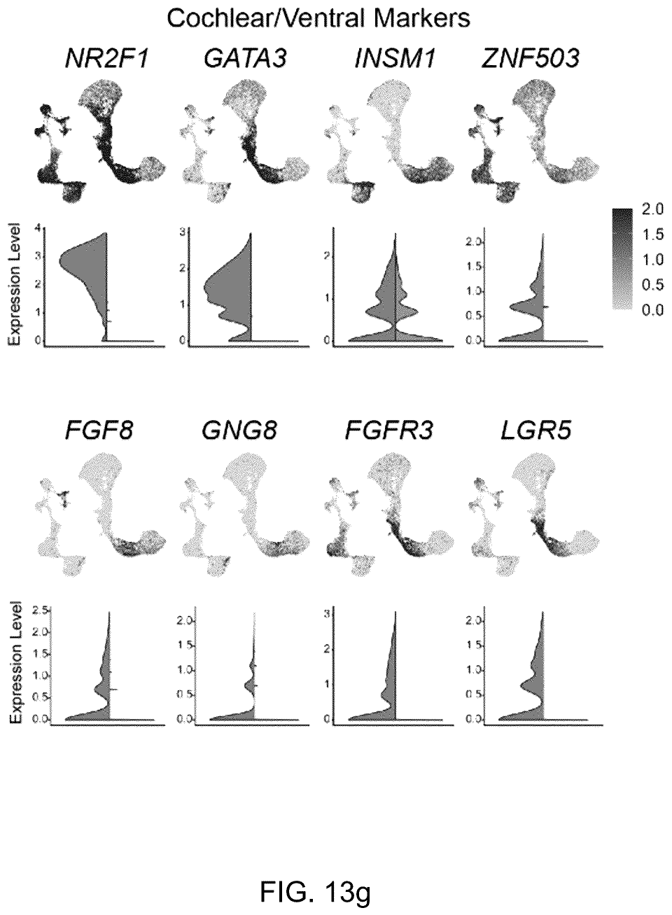

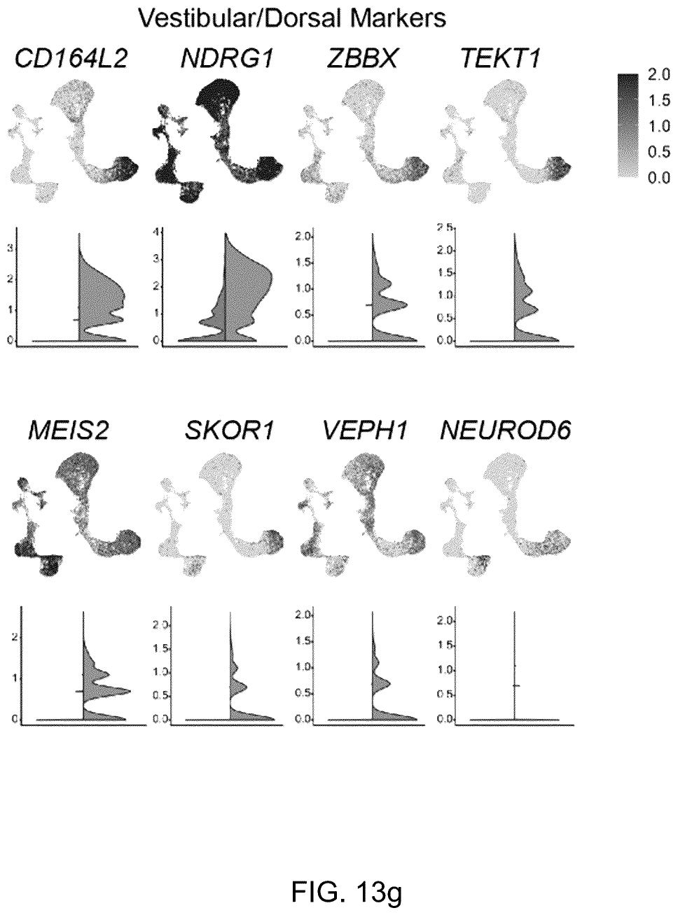

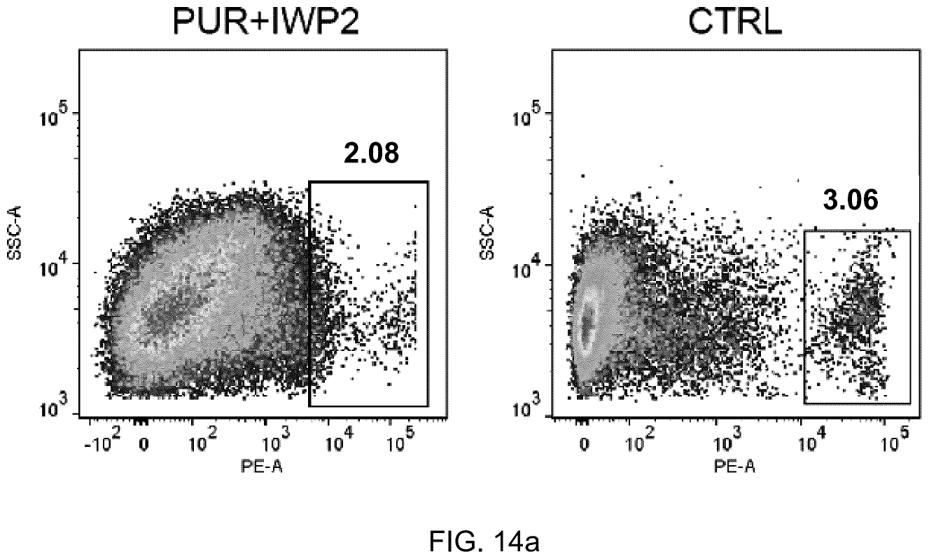

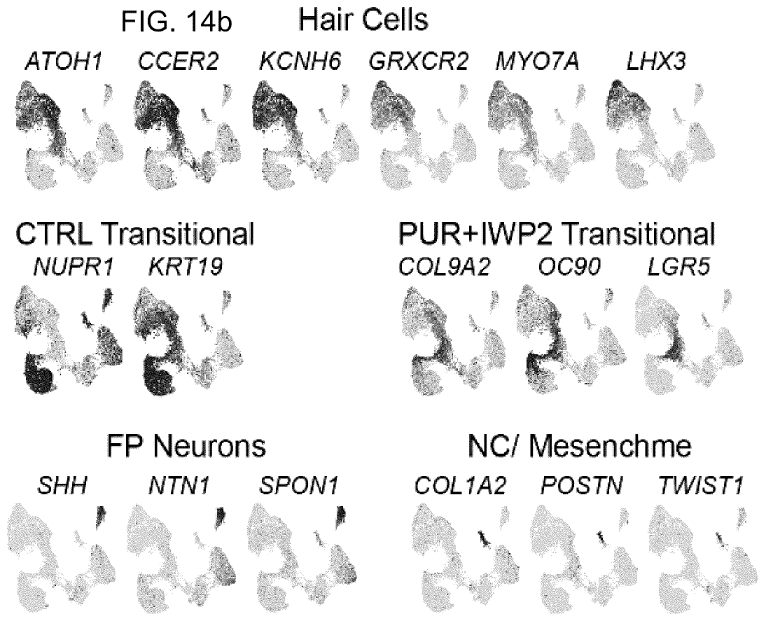

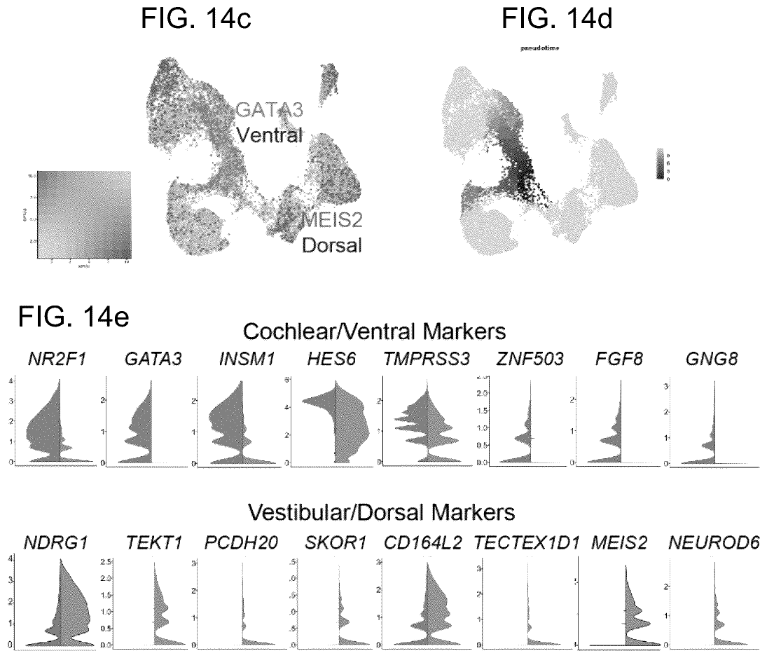

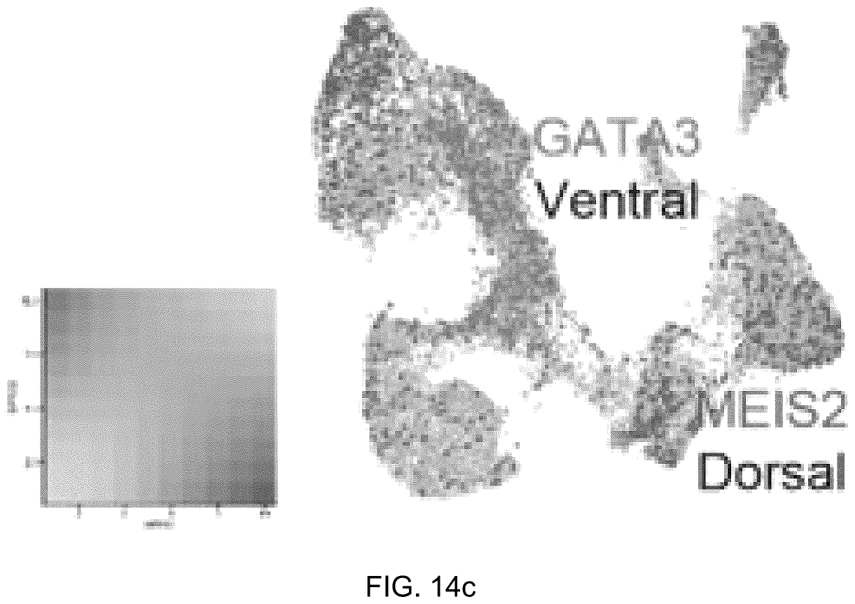

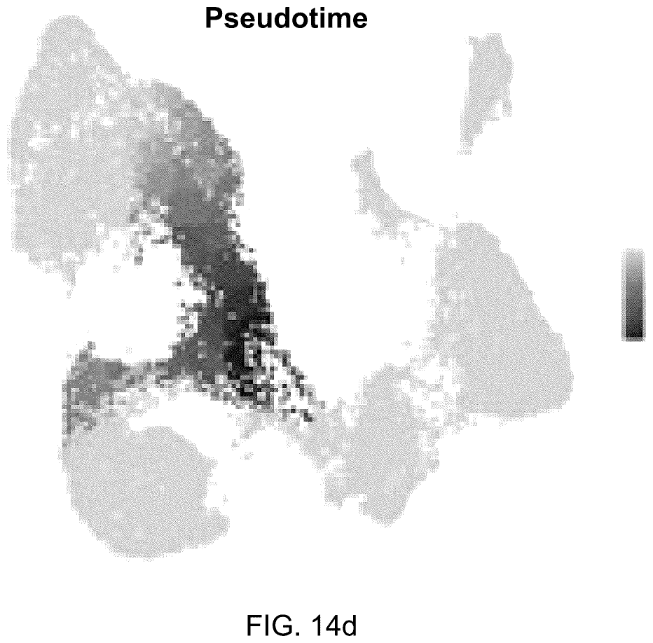

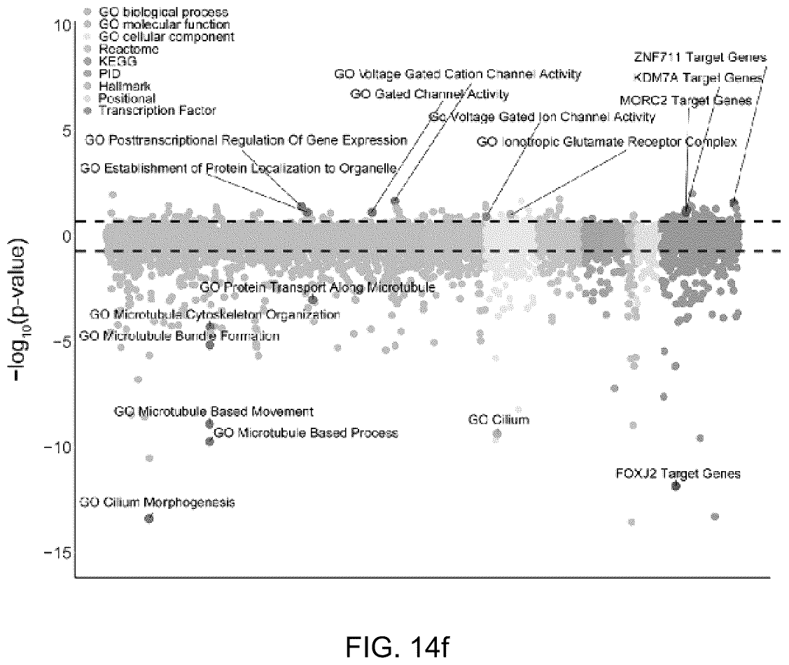

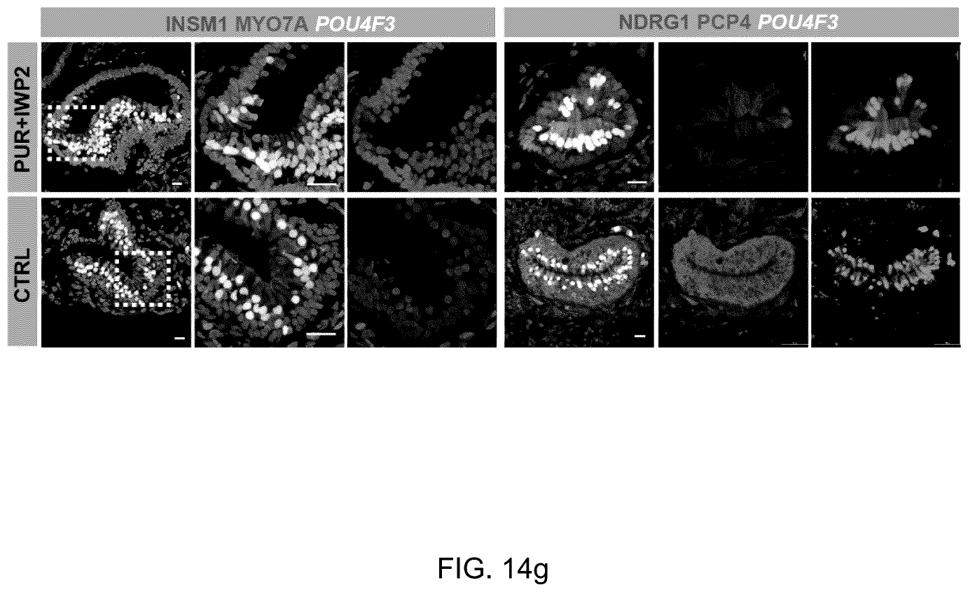

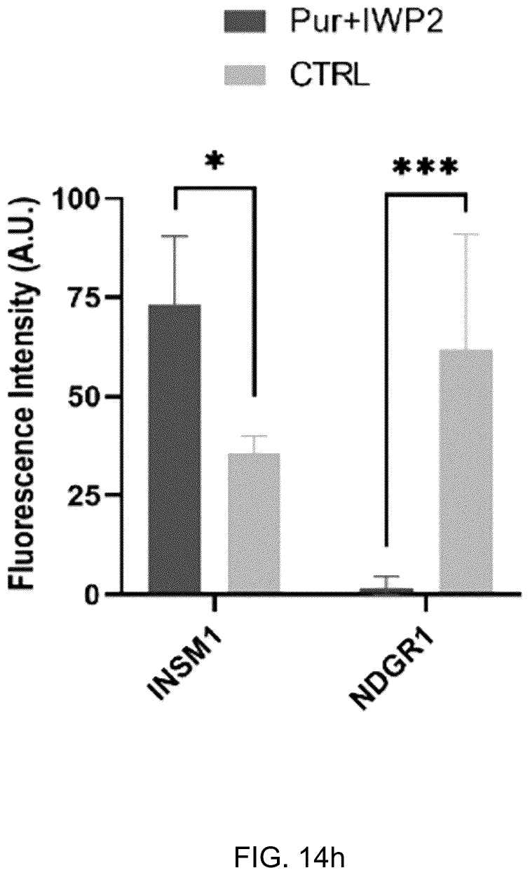

PUR+IWP2-treated and CTRL aggregates were grown in defined culture medium devoid of exogenous signalling molecules or growth factors from D22 onward. To compare the transcriptional profile between PUR+IWP2-treated and CTRL HCs, D80 and -109 POU4F3-ntdT+ sorted cells were analysed via scRNA-seq. The inventors employed a non-stringent FACS gating strategy to collect high and low POU4F3-expressing cells, which resulted in recovering sequence data from 3,332 (18.9% of total 17,668 cells) and 4,582 hair cells (28.6% of total 16,044 cells) for D80 and -109 samples, respectively. Similar to D20 scRNA-seq data, PUR+IWP2 hair cells separated out from CTRL hair cells when the cells were subjected to unbiased clustering (FIG. 4a-b; FIGS. 13-14). The volcano plot showing differentially expressed genes between the two conditions identified several known cochlear hair cell markers¹⁶⁻¹⁹, including GATA3, INSM1, HES6, TMPRSS3 and GNG8, whose expression was significantly higher in PUR+IWP2-treated vs. CTRL hair cells at D109 (FIG. 4c; FIG. 14). GSEA of PUR+IWP2 and CTRL hair cells revealed that gene sets related to voltage-gated cation channel activities are upregulated in PUR+IWP2 hair cells, whereas ciliary and microtubule-associated genes are upregulated in CTRL hair cells (FIG. 14).

Consistent with the inventors' scRNA-seq data, immunofluorescence of PUR+IWP2-treated organoids showed that POU4F3+ cells express significantly higher levels of cochlear hair cell marker proteins, such as NR2F1 and GATA3 (FIG. 4e-f), which was confirmed in hair cells of a fetal human cochlea at gestation week (GW) 18 (FIG. 4g).

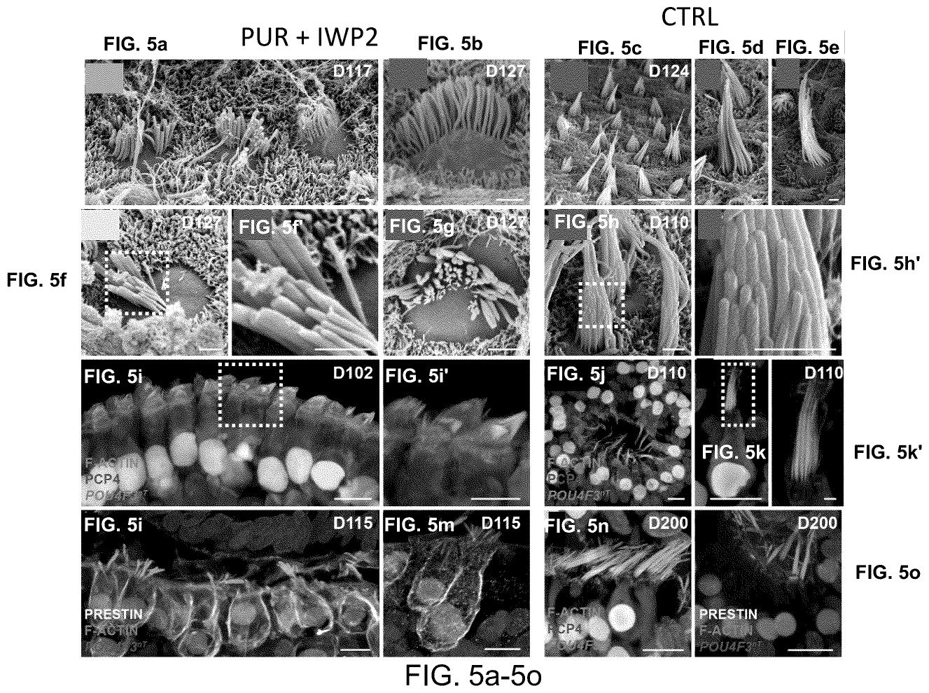

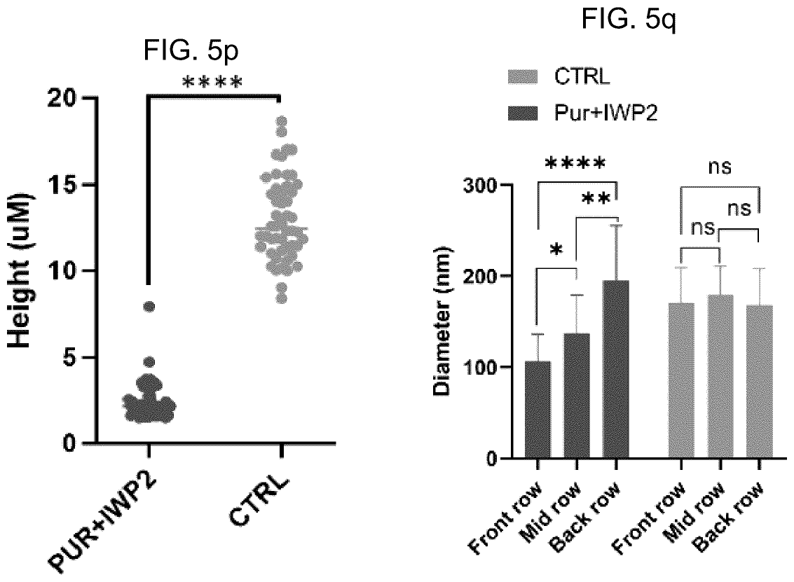

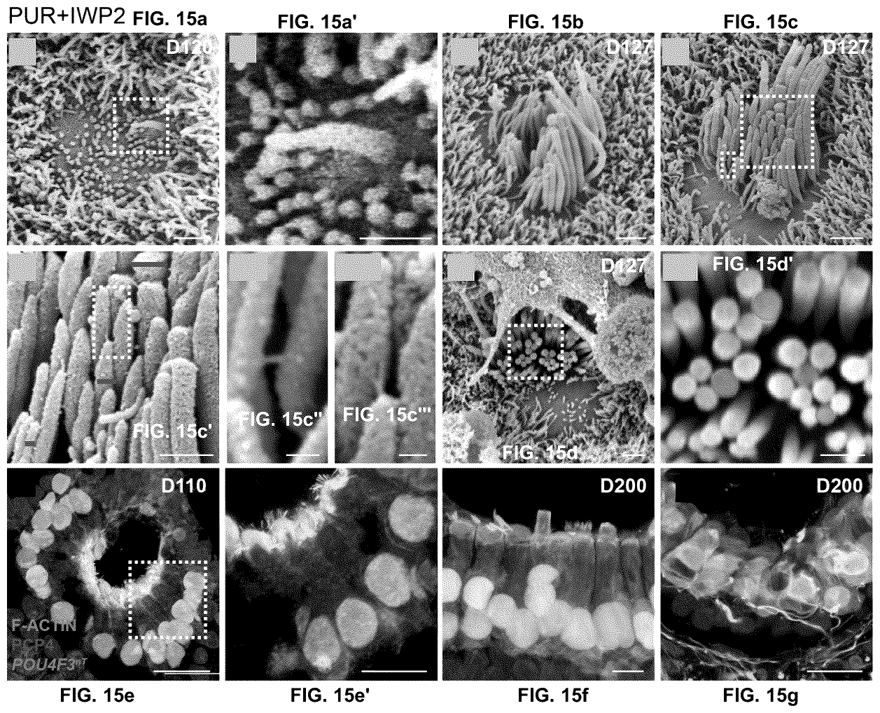

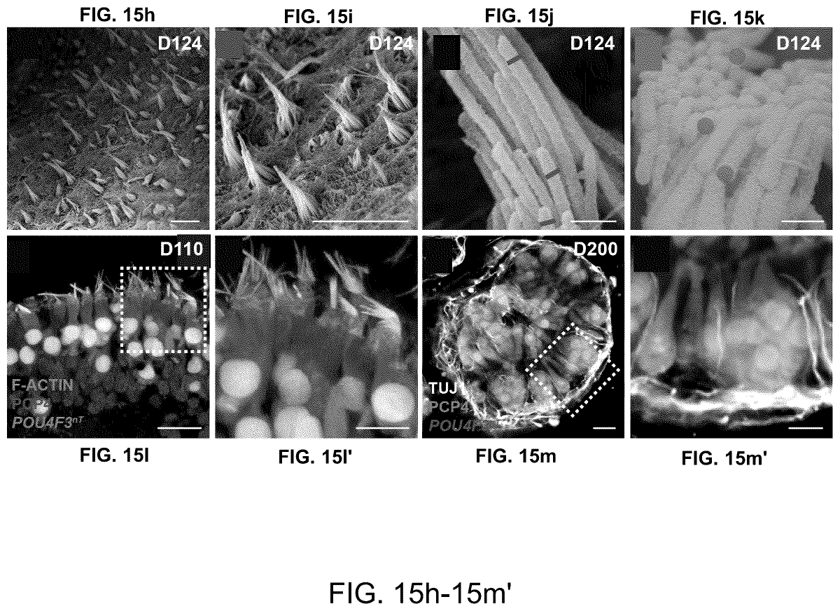

To compare the structural properties of hair bundles on the apical surface of derived hair cells between PUR+IWP2 and CTRL organoids, scanning electron microscopy was performed (FIG. 5a-h). Structural development of the hair bundle—from the emergence of a kinocilium in the middle of the cuticular plate to development of a stair-cased pattern of stereocilia²⁰, typically seen in the mouse inner ear, was observed in organoid hair cells (FIG. 15). The inventors also detected tip-link-like structures between individual stereocilia (FIG. 15). These transitional properties recapitulate development of the hair bundle in mouse cochleae. In CTRL organoids at D110-200, the inventors consistently observed long pointed arrangement of hair bundles, characteristic of vestibular hair cells. In striking contrast, the length of hair bundles was significantly shorter in PUR+IWP2-treated samples and stereocilia were often arranged in a linear or concave geometry, reminiscent of stereocilia on the apical surface of inner hair cells in the mouse cochlea (FIG. 5a-h; FIG. 15). High-resolution confocal microscopy substantiated these results by showing differences in the arrangement and overall length of stereocilia between PUR+IWP2-treated and untreated CTRL hair cells (FIG. 5i-1). Moreover, the diameter and length of individual stereocilia were positively correlated in PUR+IWP2-treated hair bundles. In contrast, the diameter of individual CTRL stereocilia was uniform along their varying lengths (FIG. 5m; FIG. 15). These structural differences in the hair bundle between PUR+IWP2 and control hair cells are consistent with structural differences observed between cochlear and vestibular hair cells in the mammalian inner ear.

To assess the identity of PUR+IWP2-treated hair cells, the inventors tested expression of PRESTIN²¹, a hallmark of cochlear outer hair cells. Membrane-localized PRESTIN is detectible in some samples as early as D102 (0.33% of HCs), and expression becomes more widespread as cultures age. On D150 and 200, 12.6% and 16.8% of all PUR+IWP2-treated hair cells, respectively, express membrane-localized PRESTIN (FIG. 6a-b). In contrast, PRESTIN was undetectable in any CTRL hair cells, up to D200 (FIG. 6a). To assess functional development of the hESC-derived hair cells, the inventors performed conventional whole cell patch clamp recordings of the voltage-activated ion conductances in the cells exhibiting strong tdTomato reporter (FIG. 6c). Two functionally distinct types of cells were identified with K⁺-based intracellular solution. The cells of the first group (type A) were observed more often (78%) and were characterized by fast outward currents and virtually no inward currents (FIG. 6d, f), similar to the mature cochlear outer hair cells²². Less frequent (22%) cells (type B) exhibited slow outward currents (FIG. 6e, h) and prominent fast inward currents (FIG. 6g), reminiscent of K⁺ and Na currents in immature inner hair cells²². Both types of cells had similar reversal potential (mean=−34.7 mV, range from −17.5 to −55.5 mV) and showed no evidence of KCNQ-type fast K⁺ current activated at resting potential 22.

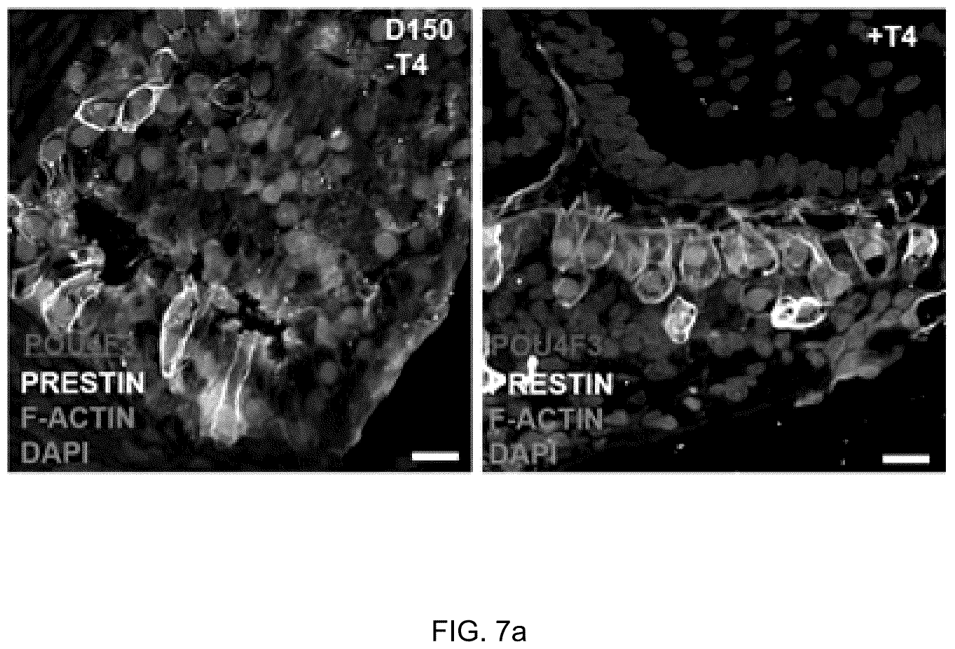

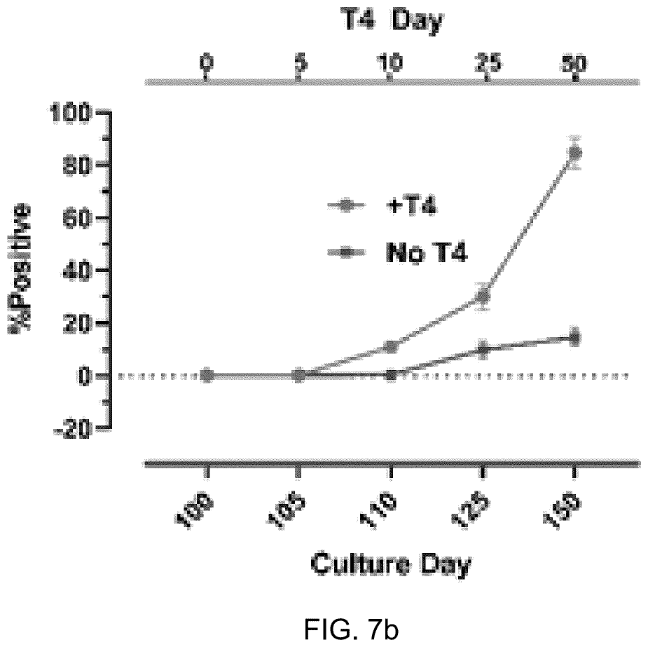

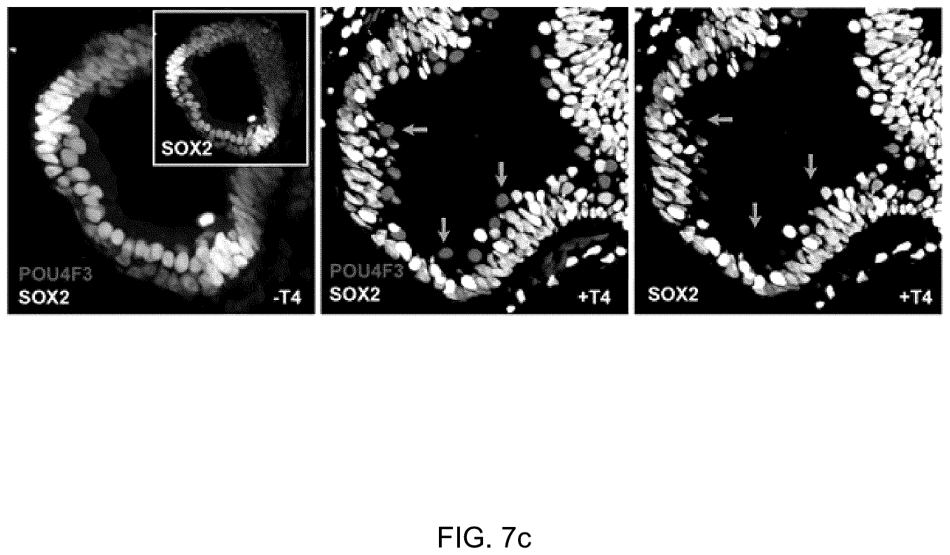

Previous studies have shown that thyroid hormones are essential for hair cell maturation and that post-natal substitution with thyroxine rescues an inner ear phenotype in PAX8 deficient mice³¹. Additionally, thyroid hormone has been shown to regulate PRESTIN expression directly through a thyroxine responsive element in the slc26a5 promotor region³². The inventors reasoned that delayed onset of PRESTIN expression and incomplete HC maturation in the inventors' cochlear organoid cultures may be due, at least in part, to hypothyroid culture conditions. Cultures treated with 250 ng/ml T4 from culture day 50 (around the first appearance of HCs in cochlear culture) until D100 had far more PRESTIN+HCs than un-supplemented cultures at D100 or even D150 (˜80% vs. ˜ 1% vs. ˜ 13%) (FIG. 6a, FIG. 7a, b). Further, SOX2 expression was also downregulated in T4 treated HCs (FIG. 7c) but not supporting cells or un-supplemented HCs suggesting that thyroid hormone signalling is also required for maturation of cochlear organoid cultures.

### Discussion

Existing protocols for inner ear organoids yield exclusively hair cells with structural, molecular, and physiological properties resembling those of native vestibular hair cells. This experimental example, on the other hand, demonstrates the development of a means to derive cochlear hair cell types in organoids. Since a gradient SHH concentration across the dorsoventral axis of otic vesicles is considered a primary ventralizing cue during inner ear development, the inventors first examined the effects of the potent smoothened agonist PUR on changes in gene expression in D20 organoids. Augmentation of the SHH pathway alone was not sufficient for promoting ventral otic marker expression. To further potentiate ventralizing conditions, the inventors applied the PKA inhibitor H89, as previous reports suggest PKA activity to be a mediator of the SHH pathway. Although a combined treatment with PUR and H89 promoted expression of the ventral otic markers, OTX2 and GATA3, this treatment greatly reduced the efficiency of deriving POU4F3+ hair cells, forcing the inventors to abandon this approach. In parallel, the inventors co-treated hESC-derived aggregates with the WNT inhibitor IWP2 beginning five days into treatment with PUR. This treatment resulted in a significant upregulation of ventral otic markers, while suppressing dorsal otic markers. The inventors also observed significant upregulation of SHH down-stream effectors, such as SULF1, LRP2, GAS1 and PTCH1, in PUR+IWP2-treated samples, but not in CTLR or PUR-treated samples, suggesting that ventralization of otic progenitors requires a threshold of SHH pathway activation that includes upregulation of ligand receptors and down-stream effectors that are antagonized by WNT signaling.

The inventors' scRNA-seq analyses of D80 and -109 samples revealed that PUR+IWP2 treated and untreated control hair cells exhibit distinctive transcriptional profiles. Of the differentially expressed genes between these two cell populations, NR2F1 and GATA3 were among the most highly expressed in PUR+IWP2 treated hair cells at both D80 and -109, suggesting these two genes as potential candidates for core elements in the transcriptional pathway leading to cochlear differentiation. GATA3 has been shown to be predominantly expressed in cochlear vs. vestibular tissues^{23,24}, but less is known about the role for the orphan nuclear receptors NR2F1/2 in cochlear specification. Targeted inactivation of NR2F1 results in supernumerary hair cells and supporting cells in the developing mouse cochlea, which is associated with dysregulation of Notch signaling components²⁵. NR2F1/2 are thought to function as ligand-dependent transcription factors, and as such might act as master drivers to confer multipotent otic progenitors with competence to differentiate into cochlear cell types. It is worth noting, however, that NR2F1/2 have been shown to play an essential role in dorso-ventral patterning of the optic vesicle through direct regulation of OTX2 expression²⁶.

The inventors sought to further classify derived hair cells in ventralized inner ear organoids as either outer or inner cochlear hair cells. Structurally, these hair cells exhibit U-shaped hair bundles with short stereocilia bearing varying diameters, as typically seen in inner hair cells of the mammalian cochlea^{27,28}. However, INSM1, a Zinc finger transcription factor essential for outer hair cell differentiation in the mouse²⁹, is predominantly expressed in hair cells of PUR+IWP2 treated organoids, while expression of TBX2, a pioneering factor for inner hair cell differentiation³⁰ is not significantly different between PUR+IWP2 and control hair cells. Additionally, the inventors' electrophysiological recordings identified two distinctive hair cell populations in D138-164 PUR+IWP2 organoids. Approximately 78% of these hair cells were characterized by fast outward currents and virtually no inward currents, which is similar to native outer but not inner hair cells in the mouse cochlea. This seems to conflict with the lower observed proportion of hair cells expressing the outer hair cell marker PRESTIN. scRNA-seq at D109 revealed that only 1.6% of PUR+IWP2 hair cells express SLC26A5, which encodes PRESTIN, and only 6.4% of these hair cells express SLC17A8, which encodes VGLUT3, an inner hair cell marker³¹. Since the proportion of PRESTIN hair cells increases over time in PUR+IWP2 organoids from D102 to 200 (from 0.33% to 16.8%), the inventors' results collectively suggest that many derived hair cells did not reach complete phenotypic maturation when the scRNA-seq experiment was performed.

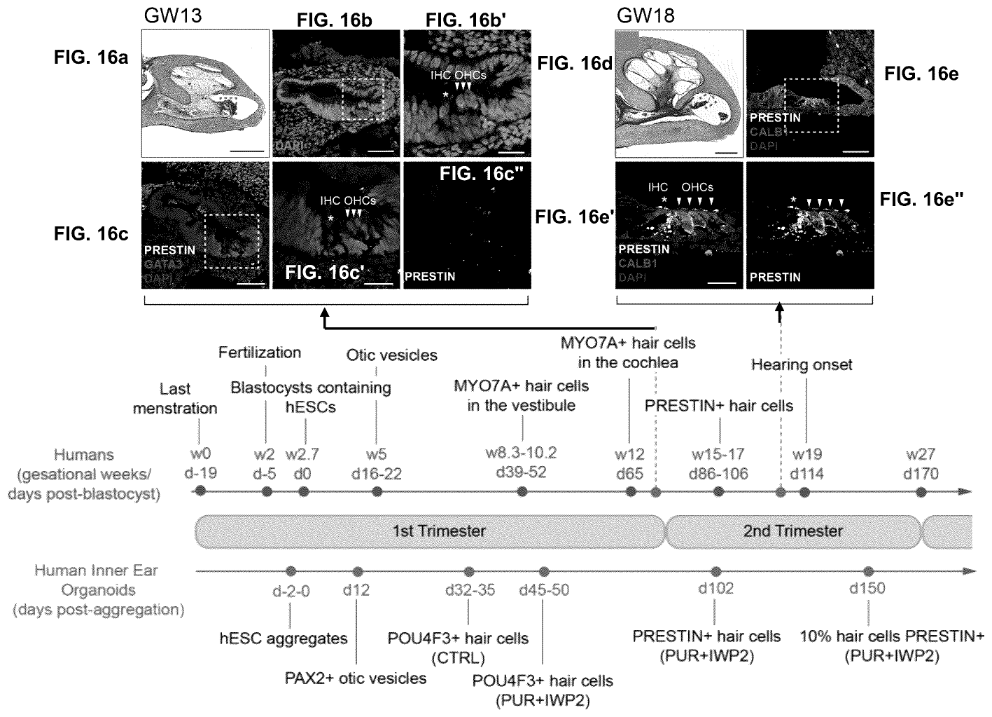

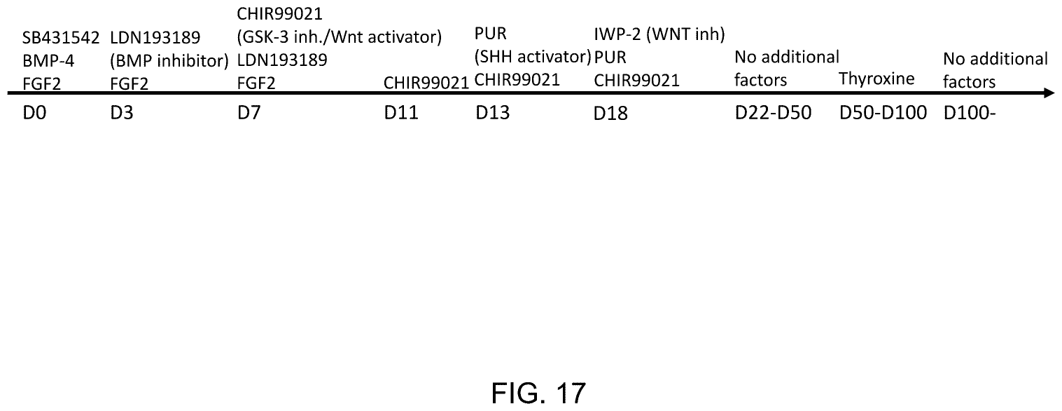

Based on the inventors' transcriptional and structural analysis, the time course of hESC-derived cochlear organoid development closely parallels that of human cochlear development, with D110 cochlear organoids corresponding approximately to human fetal cochleae at GW18 (FIG. 16). Moreover, the order of marker expression is also faithfully recapitulated in cochlear organoids with ATOHI expression preceding MYO7A expression. PRESTIN is detected in outer hair cells of the human fetal cochlea at GW18 but absent at GW 13. Likewise, PRESTIN is detected in approximately 16% of hair cells in PUR+IWP2 treated organoids at D200 but is barely detectable at D102. These results suggest that the cochlear organoids the inventors have established in the present study can reach a developmental stage comparable to the human cochlea well into the third trimester.

The inventors have demonstrated that modulations of the SHH and WNT pathways confer multipotent otic progenitors with a ventral otic phenotype, some of which subsequently give rise to hair cells with structural, transcriptional and functional properties of the two types of cochlear hair cells, inner and outer hair cells and mature in vitro well into a stage corresponding the third trimester of human fetal development. Additionally, scRNA-seq analysis identifies NR2F1 as a previously unrecognized candidate for the key transcriptional pathway essential for cochlear and vestibular diversification. Further investigation is required to elucidate the mechanisms underlying the cross talk between the transcriptional pathways and structural development and establish a means to control inner vs. outer hair cell generation. The cochlear organoids the inventors have established in this study are expected to serve as a powerful human model to investigate the biology of human cochlear development, elucidate pathogenesis of hereditary hearing loss, and identify therapeutic targets to treat profound hearing loss.

### Materials and Methods

Establishment of PAX2 POU4F3 Reporter hESC Lines with CRISPR/Cas9

To monitor the derivation of otic progenitors in 3D culture, the inventors generated PAX2 reporter hESC lines by integrating a 2A-eGFP-nls (2A-nGFP) fluorescence reporter at the endogenous PAX2 locus using the CRISPR/Cas9 genome engineering technology. Based on the locations of the stop codons, PAX2 splicing variants can be grouped as Types PAX2b, PAX2c, and PAX2d³²⁻³⁴. PCR amplification was performed on a cDNA library from D40 human inner ear organoids, as well as on PAX2b, PAX2c, and PAX2d synthetic cDNA gBlocks (IDT) for size reference. Agarose gel electrophoresis of the PCR amplicons followed by Sanger sequencing verification of the extracted brightest band showed that PAX2b was the most abundant type of PAX2 isoforms in human inner ear organoids. Therefore, gRNA and homology arms were designed to target the PAX2b stop codon locus. To construct the PAX2-2A-eGFP-nls-pA-loxP-PGK-Puro-pA-loxP donor vector, two 1 kb homology arms flanking the PAX2b stop codon were PCR amplified from WA25 hESC (WiCell) genomic DNA. Gibson assembly³⁵ was used to connect the two homology arms with 2A-eGFP^{8,36}, nls-stop-bGH polyA (gBlock, IDT), and loxP-PGK-Puro-pA-loxP DNA fragments^{8,36} as well as a linearized pUC19 plasmid backbone into the final donor vector. gRNA (5′-ATGACCGCCACTAGTTACCG-3′ (SEQ ID NO: 29)) targeting the PAX2b stop codon was cloned into an expression plasmid under the control of a U6 promoter (Addgene #71814)³⁷. The PAX2-2A-nGFP donor vector, the PAX2b gRNA plasmid, as well as a high fidelity Cas9 expression plasmid (SpCas9-HF1, Addgene #72247)⁹ were transfected into WA25 hESCs with 4D Nucleofector (Lonza) using the P3 Primary Cell 4D-Nucleofector X kit and Program CB-150. After nucleofection, cells were plated in E8fn medium containing 1× RevitaCell (Thermo Fisher) for improved cell survival rate, and 1 μM of Scr7 (Xcessbio) for higher HDR efficiency³⁸. 0.5 μg/mL puromycin selection was performed for 14 days starting from 48 h post-nucleofection. The PGK-Puro sub-cassette flanked by two loxP sites was removed from the genome after puromycin selection by nucleofection of a Cre recombinase expressing vector (Addgene #13775). Clonal cell lines were established by low-density seeding (1-3 cells/cm²) of dissociated single hESCs followed by isolation of hESC colonies after 5-7 d of expansion. Genotypes of the clonal cell lines were analysed by PCR amplification followed by gel electrophoresis, and by Sanger sequencing of total PCR amplicons or individual PCR amplicons cloned into TOPO vectors (Thermo Fisher). Cell lines with bi-allelic 2A-nGFP integration were used for subsequent experiments. Top 10 predicted off-target sites of the gRNA were PCR amplified (˜1 kb) from the genomic DNA of the established cell lines and were Sanger sequenced to test for off-target mutations.

To build a PAX2-2A-nGFP/POU4F3-2A-ntdTomato multiplex reporter cell line, the POU4F3-2A-ntdTomato knockin CRISPR was performed on the PAX2-2A-nGFP genetic background. The POU4F3-2A-tdTomato-nls-bGH polyA-frt-PGK-Puro-pA-frt donor plasmid was constructed by connecting the following DNA fragments via Gibson assembly: Two 1 kb homology arms flanking the POU4F3 stop codon amplified from WA25 genomic DNA, 2A-tdTomato gBlock DNA, nls-stop-bGH pA gBlock DNA, frt-PGK-Puro-frt gBlock DNA (IDT), and a linearized pUC19 backbone. The completed donor plasmid was transfected into the PAX2-2A-nGFP parental hES cell line along with a ribonucleoprotein complex composed of a synthetic sgRNA (5′-ATTCGGCTGTCCACTGATTG-3′ (SEQ ID NO: 30)) (Synthego) targeting the POU4F3 stop codon locus, and a high fidelity Cas9 protein (HiFi Cas9 v3, IDT). The transfection was also performed with a 4D Nucleofector (Lonza) using the P3 Primary Cell 4D-Nucleofector X kit and Program CB-150. After nucleofection, cells were plated in E8fn medium containing 1× RevitaCell (Thermo Fisher) and 1 μM of Scr7 (Xcessbio). 0.5 μg/mL puromycin selection was performed for 9 days starting from 48 h post-nucleofection. The frt-flanked PGK-Puro cassette was removed via FLPo transfection (Addgene #13793). Clonal cell line isolation and genotyping procedures were the same as PAX2-2A-nGFP CRISPR. The final multiplex cell line chosen for downstream experiments had bi-allelic 2A-nGFP knockin at the PAX2 locus and bi-allelic 2A-ntdTomato knockin at the POU4F3 locus, and was karyotyped by the KaryoLogic Inc. (Research Triangle Park, North Carolina).

### Organoid Culture

PAX2-2A-nGFP/POU4F3-2A-ntdTomat hESCs (passages 22-50) were maintained and passaged in Essential 8 Flex Medium (Thermo Fisher, A2858501) supplemented with 100 μg/ml Normocin (E8fn) on recombinant human Vitronectin-N(Thermo Fisher, A14700)-coated 6-well plates. Human inner ear organoids were derived from hESCs based on the inventors' previous protocol 8.10 with major modifications. Briefly, hESCs were dissociated with StemPro Accutase (Thermo Fisher, A1110501) and distributed at 3500 cells per well onto low-adhesion 96-well U-bottom plates in 100 μL E8fn containing 20 μM Y-27632 (Stemgent, 04-0012-02) and forcibly aggregated at 120 g for 5 minutes. 100 μL of E8fn was added to each well after >4 h of incubation. Induction of non-neural epithelium occurred following 48 h of aggregation, marking differentiation day 0, as follows: aggregates were washed extensively in DMEM:F12 with HEPES buffer (DFH) (Thermo Fisher, 11320033) and transferred to new 96-well U-bottom plates in 100 μL of E6 medium (Thermo Fisher, A1516401) with Normocin (E6n) containing 4 ng mL⁻¹ FGF-2 (StemCell Technologies, 780003), 10 μM SB-431542 (Stemgent, 04-0010-05), and 2% growth factor reduced (GFR) Matrigel (Corning, 354230). On day 3 of differentiation, 25 μL of E6n containing 50 ng/ml FGF-2 and 200 nM LDN-193189 (Stemgent, 04-0074-02) was added to each well. Culture medium was changed on days 7 and 9 with Eon containing 3 μM CHIR 99021 (Reprocell, 04-0004-10), 200 nM LDN, and 50 ng/mL FGF-2. On day 11, aggregates were washed and transferred to a Nunc Delta surface 6-well culture dish in Organoid Maturation Medium (OMM) containing 1% GFR Matrigel and 3 μM CHIR99021. OMM consists of a 50:50 mixture of Advanced DMEM:F12 (Thermo Fisher, 12634028) and Neurobasal Medium (Thermo Fisher, 21103049) supplemented with 0.5×N2 Supplement (Thermo Fisher, 17502048), 0.5×B27 minus Vitamin A (Thermo Fisher, 12587010), 1× GlutaMAX (Thermo Fisher, 35050061), 0.1 mM β-Mercaptoethanol (Thermo Fisher, 21985023), and Normocin. Control cultures were maintained in OMM+3 μM CHIR99021 until day 18 with media changes on days 13 and 15, then OMM for the remainder of the culture. Treatment cultures were supplemented with 1 μM Purmorphamine (Stemgent, 04-0009) from days 13-22, and IWP-2 (Tocris, 3533) from days 18-22, with media changes on days 15,18, and 20, then maintained in OMM for the remainder of the culture.

### Immunohistochemistry

Aggregates were fixed with 4% paraformaldehyde for 30 min at room temperature or at 4° C. overnight. The fixed specimens were cryoprotected with a graded series of sucrose and then embedded in tissue-freezing medium. Frozen tissue blocks were sectioned into 12-μm cryosections on a Leica CM-1860 cryostat. For immunostaining, a 10% goat or horse serum in 0.1% Triton X-100 1×PBS solution was used for blocking, and a 3% goat or horse serum in 0.1-1% Triton X-100 1×PBS solution was used for primary/secondary antibody incubations. Primary antibodies used in this study are listed in Table 1. Alexa Fluor conjugated anti-mouse, rabbit, or goat IgG (Thermo Fisher) were used as secondary antibodies. ProLong Gold Antifade Reagent with DAPI (Thermo Fisher) was used to mount the samples and visualize cellular nuclei.

For whole-mount immunofluorescence, the AbScale tissue-clearing protocol³⁹ was applied to aggregate samples. Incubation and washing steps were performed on a rotor, and incubation steps were performed at 37° C. on a rotor unless otherwise noted. After fixation of samples with 4% paraformaldehyde overnight, the samples were incubated for 6 h in Scale SO solution, followed by incubation in Scale A2 for 16 h, ScaleB4 solution for 24 h, and ScaleA2 solution for 8 h. Thereafter the samples were incubated in 0.1 M PBS for 4 h at room temperature, and blocking was performed with 10% normal horse serum (Vector Laboratories) in AbScale solution for 16 h. Incubation with primary antibodies in AbScale solution containing 3% normal horse serum was performed for 48 h, followed by sequential washing with AbScale solution for 15, 30, 60, and 120 min and incubation with fluorophore-conjugated secondary antibodies in AbScale solution containing 3% normal horse serum for 24 h. After rinsing three times with AbScale at RT for 30 min and twice with AbScale Rinse solution at RT for 30 min, the samples were re-fixed with 4% paraformaldehyde at RT for 1 h, washed twice with 0.1 M PBS, and incubated in ScaleS4 solution for 16 h. The stained samples were mounted with a small amount of ScaleS4 solution on poly-L-ornithine coated coverslips with silicone gaskets (Fisher Scientific). Imaging of the samples was carried out on a Leica Dive Confocal/Multiphoton Microscope or a Nikon A1R HD25 confocal microscope. Three-dimensional reconstructions were performed using the Imaris 8 software package (Bitplane) and the NIS Elements Advanced Research application (Nikon).

### Quantification of Immunohistochemistry Data

Treated and control samples were processed for immunostaining simultaneously using identical reagents and protocols for each comparison. Images were acquired using either a Nikon AIR-HD25 confocal microscope, or a Leica DMI8 widefield fluorescent microscope using identical image acquisition parameters between conditions. Raw images were analysed using Nikon GI3 suite or exported in TIFF format and analysed using ImageJ. Co-expression analysis was performed by analysing regions for coincident signal corresponding to a labelled tissue-specific marker and a gene of interest. Mean grey value and total colocalized area were collected.

Statistical analysis was performed in GraphPad Prism 9. All datasets were analysed using a two-sided Welch's t-test. Data collection and analysis were not performed blinded.

### Scanning Electron Microscopy

Aggregates with abundant POU4F3-ntdTomato+ puncta between D81 and 141 of differentiation were fixed with 2.5% glutaraldehyde in sodium cacodylate buffer (Electron Microscopy Sciences) at 4° C. overnight. The fixed samples were dissected under a Nikon SMZ18 stereo fluorescence microscope to expose the luminal surface of vesicles containing ntdTomato+ cells and post-fixed with 1% osmium tetroxide (Electron Microscopy Sciences) at RT for 1 h. The samples were then dehydrated through a graded series of ethanol and transferred into a Leica EM CPD300 critical-point dryer. After critical point drying, the samples were mounted on aluminium stubs and sputter coated with a Denton Vacuum Desk V sample preparation system. The samples were viewed on a JEOL JSM-7800F filed emission scanning electron microscope at an accelerating voltage of 5 kV.

### Representative Data and Reproducibility

Unless stated otherwise, images are representative of specimens obtained from at least three separate experiments. For immunohistochemical analysis of aggregates, the inventors typically sectioned 6-15 aggregates from each condition in each experiment.

scRNA-Seq and Raw Data Processing

PAX2^{nG}+ (D20 samples) or POU4F3^{nT}+ (D80 and -109 samples) cells were isolated and used for scRNA-seq analyses. Thirty to 45 aggregates per condition were washed with 0.5 mM EDTA (Thermo Fisher) in DPBS, then incubated with 1.1 mM EDTA in 1× TrypLE (Life Technologies) in DPBS on an orbital shaker at 37° C. for 30 min followed by 40-50 min without shaking. During incubation, the samples were mechanically dissociated on Nunclon Sphera 24-well plates (Thermo Fisher) with shaking and occasional gentle pipetting with P1000 tips. After confirming that most cells, especially those with reporter expression, were completely dissociated, they were filtered sequentially through a 100-μm and a 40-μm cell strainer (Falcon) and then transferred into 2-ml tubes (Eppendorf). After spinning down at 100×g for 5 min, dissociated cells were resuspended in FACS buffer consisting of 2% fetal bovine serum (Thermo Fisher) in 1×DPBS. Cells were again filtered through a 40-μm cell strainer and collected into a 5-ml round-bottom polypropylene tube (Falcon). Dead cells were stained with propidium iodide (PI, Invitrogen) diluted at 1:500. Simultaneously, size control cells without PI staining were prepared using aggregates made from wild-type hESCs using the above protocol. Cells were stored on ice and protected from light prior to sorting. PI-negative and GFP (or tdTomato)-positive populations were collected and cleaned using a SORP Aria (BD Biosciences).

scRNA-seq for all samples was performed using the 10× Genomics Chromium 3′ v3 platform for cDNA library construction and the NovaSeq 6000 system (Illumina) for sequencing. For each sample, between 12,000 and 18,000 cells were added to the single cell master mix, following the Chromium NextGEM Single Cell 3′ Reagent Kits User Guide. The single cell master mix, along with the single-cell gel beads and partitioning oil, was dispensed onto a Single Cell Chip G and the chip was loaded into the Chromium Controller for barcoding and cDNA synthesis. The resulting cDNA library was sequenced with the NovaSeq 6000 system running a custom program for 28-bp plus 91-bp paired-end sequencing, resulting in a read depth of more than 40,000 reads per cell.

Illumina's CellRanger v4.0.0 program was used to generate BCL files, which were de-mutiplexed and converted to FASTQ files via bcl2fastq Conversion Software (Illumina). The FASTQ files were then aligned to the GRCh38-3.0.0 reference genome using the STAR (Spliced Transcripts Alignment to a Reference) aligner. Mapped reads were grouped by cell barcode, single-cell gene expression was quantified using unique molecular identifiers (UMIs), and the resulting filtered gene-barcode (count) matrices were used as input for downstream analysis.

scRNA-Seq Data Analysis

The filtered count matrices were preprocessed individually for each dataset, removing cells where the number of mitochondrial gene counts exceeded 12.5 percent of the total number of molecules detected. A second preprocessing step removed cells with anomalous housekeeping gene expression: Cells with log-transformed RPL27 expression ±2 standard deviations away from the mean were removed. After preprocessing, datasets were merged across conditions, resulting in one combined dataset for each timepoint. After merging, the gene expression levels were normalized using the SCTransform function in Seurat⁴⁰, which transforms raw count data into Pearson residuals, effectively controlling for technical variation resulting from heterogenous sequencing depth. Over fifty different cluster partitions were generated using Seurat's unsupervised clustering workflow by varying the values of the resolution and k.param parameters required for clustering and shared nearest neighbor graph construction, respectively. Then, the cluster partition with the highest silhouette index was selected for further analysis⁴¹. Cluster identity was manually determined based on the expression of canonical markers.

Relevant populations (e.g. otic progenitors, hair cells) were isolated on the basis of cluster identity and comparisons between treatment groups were performed using DESeq2 and zingeR⁴². Cell-level weights-which are required to adjust for drop-out events endemic to scRNA-sequencing-were calculated with zingeR. Differential expression between treatment groups was determined using DESeq2, and genes were considered statistically significant with a Benjamini-Hochberg P<0.05. The results of the DESeq2 analysis were passed to iDEA¹⁵, a platform for gene set enrichment analysis, and differentially expressed genes were compared to gene sets taken from MSigDB databases⁴³. The Gene Transcription Regulation Database (GTRD) was used to define the 573 transcription factor target gene sets the inventors' data were compared against⁴⁴. While these gene sets are well defined, they do not constitute an exhaustive list of all possible transcription factors.

### Electrophysiological Analyses

D138-164 hESC-derived organoids were first imaged in bright field and epifluorescent illumination (TE2000-U, Nikon) to determine localization of otic vesicles with tdTomato positive cells. Then, the organoids were sectioned with a diamond knife to expose otic vesicles. The section was placed into the custom-made recording chamber, where it was held by two strands of dental floss. The recordings were performed at room temperature (20-25° C.) in Leibovitz's L-15 cell culture medium (Cat #21083027, Gibco/ThermoFisher, USA) containing the following inorganic salts (in mM): NaCl (137), KCl (5.4), CaCl2 (1.26), MgCl2 (1.0), Na2HPO4 (1.0), KH2PO4 (0.44), MgSO4 (0.81). The cells were viewed with an upright microscope (E600FN, Nikon), equipped with a high numerical aperture (NA) objective (60×, 1.0 NA) and epifluoresent attachment. Only the cells with bright tdTomato signal were chosen for recordings. During recordings, the organoid sections were continuously perfused with L-15 medium. Pipettes for whole-cell patch-clamp recordings were filled with intracellular solution containing (in mM): KCl (12.6), KGlu (131.4), MgCl2 (2), EGTA (0.5), K2HPO4 (8), KH2PO4 (2), Mg2-ATP (2), and Na4-GTP (0.2). The solution was adjusted to pH 7.3-7.4 with KOH and to 320 mOsm with D-glucose. The uncompensated pipette resistance was typically 5-8 MOhm when measured in the bath. Whole cell current responses were recorded with a MultiClamp 700B patch clamp amplifier controlled by pClamp software (Molecular Devices, USA).

### Human Tissue Ethics, Collection and Immunofluorescence

Human fetal cochleae were collected and processed at the Leiden University Medical Center (Netherland) from tissues obtained from elective abortion using vacuum aspiration. Prior to the procedure, obstetric ultrasonography was performed to determine the gestational weeks (GW) and days. One cochlea at GW 13 and another at GW 18 were used in this study. The fresh samples were collected in PBS, fixed in 4% paraformaldehyde in PBS overnight at 4° C., decalcified and embedded in paraffin as previously described⁴⁵. The paraffin blocks were cut into 5 μm thick sections through the sagittal plane using a Leica rotary microtome. The serial sections were deparaffinized in xylene, rehydrated in a descending ethanol series (100%, 90%, 70%, 50%), and rinsed in distilled water. Thereafter, the sections were treated in 0.01 M sodium citrate buffer (pH 6.0) for 12 minutes at 98° C. using a boiling pot for antigen-retrieval and then processed for immunofluorescence. Primary antibodies used in this study are listed in Table 1. The stained samples were viewed and imaged on a Zeiss LSM900 confocal microscope. The use of human fetal tissues was approved by the Medical Ethical Committee of the Leiden University Medical Center (Protocol Number 08.087). Informed written consents were obtained in accordance with the WMA Declaration of Helsinki guidelines.

TABLE 1
Antibodies used in Example 1.

| | | | | | dilu- |
|---|---|---|---|---|---|
| antibody | host | isotype | supplier | cat. No. | tion |
| CALB1 | mouse | IgG1 | SWANT | 300 | 1:100 |
| DLX3 | mouse | IgG1 | Santa Cruz | sc-514094 | 1:100 |
| FBXO2 | mouse | IgG1 | Santa Cruz | sc-398111 | 1:25 |
| GATA3 | goat | IgG | R&D Bio | AF2605 | 1:100 |
| GFP | mouse | IgG2a | Thermo Fisher | A-11120 | 1:100 |
| INSM1 | mouse | IgG1 | Santa Cruz | Sc-271408 | 1:100 |
| LMOD3 | rabbit | IgG | Proteintech | 14948-1-AP | 1:100 |
| MYO7A | rabbit | IgG | Proteus | 256790 | 1:100 |
| MYO7A | mouse | IgG2a | Santa Cruz | sc-74516 | 1:20 |
| NDGR1 | rabbit | IgG | Abcam | ab-124689 | 1:100 |
| NR2F1 | rabbit | IgG | Invitrogen | ab-124689 | 1:100 |
| OCT4 | rat | IgG2a | eBioscience | 53584182 | 1:100 |
| (conjugated | | | | | |
| to AF488) | | | | | |
| OTX2 | goat | IgG | R&D Bio | AF1979 | 1:100 |
| PAX8 | rabbit | IgG | Abcam | AB97477 | 1:100 |
| PCP4 | rabbit | IgG | Santa Cruz | sc-74816 | 1:100 |
| phalloidin | NA | NA | Thermo Fisher | A12379 | 1:100 |
| (conjugated | | | | | |
| to AF488) | | | | | |
| POU4F3 | mouse | IgG1 | Santa Cruz | sc-81980 | 1:25 |
| Prestin | rabbit | IgG | Generous gift | NW-802 | 1:500 |
| | | | of Drs. Jing | | |
| | | | Zheng and | | |
| | | | Kazuaki Homma | | |
| Sox10 | mouse | IgG1 | eBioscience | 14-5923-82 | 1:50 |
| Sox2 | mouse | IgG1 | BD Pharmingen | 561469 | 1:200 |
| SSEA4 | mouse | IgG3 | BioLegend | 330414 | 1:200 |
| (conjugated | | | | | |
| to AF594) | | | | | |
| SULF1 | rabbit | IgG | Invitrogen | PA5-113112 | 1:100 |
| TUJ1 | mouse | IgG2a | Biolegend | 801201 | 1:100 |
| (TUBB3) | | | | | |

AF488, Alexa Fluor 488.
AF594, Alexa Fluor 594.
IHC, immunohistochemistry.

### REFERENCES

1 Barald, K. F. & Kelley, M. W. From placode to polarization: new tunes in inner ear development. Development 131, 4119-4130, doi: 10.1242/dev.01339 (2004).
2 Groves, A. K. & Fekete, D. M. Shaping sound in space: the regulation of inner ear patterning. Development 139, 245-257, doi: 10.1242/dev.067074 (2012).
3 Wu, D. K. & Kelley, M. W. Molecular mechanisms of inner ear development. Cold Spring Harb Perspect Biol 4, a008409, doi: 10.1101/cshperspect.a008409 (2012).
4 Morton, C. C. & Nance, W. E. Newborn hearing screening—a silent revolution. N Engl J Med 354, 2151-2164, doi: 10.1056/NEJMra050700 (2006).
5 Koehler, K. R., Mikosz, A. M., Molosh, A. I., Patel, D. & Hashino, E. Generation of inner ear sensory epithelia from pluripotent stem cells in 3D culture. Nature 500, 217-221, doi: 10.1038/nature12298 (2013).
6 Koehler, K. R. & Hashino, E. 3D mouse embryonic stem cell culture for generating inner ear organoids. Nat Protoc 9, 1229-1244, doi: 10.1038/nprot.2014.100 (2014).
7 Liu, X. P., Koehler, K. R., Mikosz, A. M., Hashino, E. & Holt, J. R. Functional development of mechanosensitive hair cells in stem cell-derived organoids parallels native vestibular hair cells. Nat Commun 7, 11508, doi: 10.1038/ncomms11508 (2016).
8 Koehler, K. R. et al. Generation of inner ear organoids containing functional hair cells from human pluripotent stem cells. Nat Biotechnol 35, 583-589, doi: 10.1038/nbt.3840 (2017).
9 Kleinstiver, B. P. et al. High-fidelity CRISPR-Cas9 nucleases with no detectable genome-wide off-target effects. Nature 529, 490-+, doi: 10.1038/nature 16526 (2016).
10 Nie, J. & Hashino, E. Generation of inner ear organoids from human pluripotent stem cells. Methods Cell Biol 159, 303-321, doi: 10.1016/bs.mcb.2020.02.006 (2020).
11 Riccomagno, M. M., Martinu, L., Mulheisen, M., Wu, D. K. & Epstein, D. J. Specification of the mammalian cochlea is dependent on Sonic hedgehog. Genes Dev 16, 2365-2378, doi: 10.1101/gad. 1013302 (2002).
12 Ohta, S., Wang, B., Mansour, S. L. & Schoenwolf, G. C. SHH ventralizes the otocyst by maintaining basal PKA activity and regulating GLI3 signaling. Dev Biol 420, 100-109, doi: 10.1016/j.ydbio.2016.10.004 (2016).
13 Ohta, S., Wang, B., Mansour, S. L. & Schoenwolf, G. C. BMP regulates regional gene expression in the dorsal otocyst through canonical and non-canonical intracellular pathways. Development 143, 2228-2237, doi: 10.1242/dev. 137133 (2016).
14 Riccomagno, M. M., Takada, S. & Epstein, D. J. Wnt-dependent regulation of inner ear morphogenesis is balanced by the opposing and supporting roles of Shh. Genes Dev 19, 1612-1623, doi: 10.1101/gad. 1303905 (2005).
15 Ma, Y. et al. Integrative differential expression and gene set enrichment analysis using summary statistics for scRNA-seq studies. Nat Commun 11, 1585, doi: 10.1038/s41467-020-15298-6 (2020).
16 Burns, J. C., Kelly, M. C., Hoa, M., Morell, R. J. & Kelley, M. W. Single-cell RNA-Seq resolves cellular complexity in sensory organs from the neonatal inner ear. Nat Commun 6, 8557, doi: 10.1038/ncomms9557 (2015).
17 Scheffer, D. I., Shen, J., Corey, D. P. & Chen, Z. Y. Gene Expression by Mouse Inner Ear Hair Cells during Development. J Neurosci 35, 6366-6380, doi: 10.1523/JNEUROSCI.5126-14.2015 (2015).
18 Kolla, L. et al. Characterization of the development of the mouse cochlear epithelium at the single cell level. Nat Commun 11, 2389, doi: 10.1038/s41467-020-16113-y (2020).
19 Elkon, R. et al. RFX transcription factors are essential for hearing in mice. Nat Commun 6, 8549, doi: 10.1038/ncomms9549 (2015).
20 Barr-Gillespie, P. G. Assembly of hair bundles, an amazing problem for cell biology. Mol Biol Cell 26, 2727-2732, doi: 10.1091/mbc.E14-04-0940 (2015).
21 Zheng, J. et al. Prestin is the motor protein of cochlear outer hair cells. Nature 405, 149-155, doi: 10.1038/35012009 (2000).
22 Ahmed, Z. M. et al. The tip-link antigen, a protein associated with the transduction complex of sensory hair cells, is protocadherin-15. J Neurosci 26, 7022-7034, doi: 10.1523/JNEUROSCI.1163-06.2006 (2006).
23 Luo, X. J. et al. GATA3 controls the specification of prosensory domain and neuronal survival in the mouse cochlea. Hum Mol Genet 22, 3609-3623, doi: 10.1093/hmg/ddt212 (2013).
24 Bardhan, T. et al. Gata3 is required for the functional maturation of inner hair cells and their innervation in the mouse cochlea. J Physiol 597, 3389-3406, doi: 10.1113/JP277997 (2019).
25 Tang, L. S., Alger, H. M. & Pereira, F. A. COUP-TFI controls Notch regulation of hair cell and support cell differentiation. Development 133, 3683-3693, doi: 10.1242/dev.02536 (2006).
26 Tang, K. et al. COUP-TFs regulate eye development by controlling factors essential for optic vesicle morphogenesis. Development 137, 725-734, doi: 10.1242/dev.040568 (2010).
27 Velez-Ortega, A. C., Freeman, M. J., Indzhykulian, A. A., Grossheim, J. M. & Frolenkov, G. I. Mechanotransduction current is essential for stability of the transducing stereocilia in mammalian auditory hair cells. Elife 6, doi: 10.7554/eLife.24661 (2017).
28 Hadi, S., Alexander, A. J., Velez-Ortega, A. C. & Frolenkov, G. I. Myosin-XVa Controls Both Staircase Architecture and Diameter Gradation of Stereocilia Rows in the Auditory Hair Cell Bundles. J Assoc Res Otolaryngol 21, 121-135, doi: 10.1007/s10162-020-00745-4 (2020).
29 Wiwatpanit, T. et al. Trans-differentiation of outer hair cells into inner hair cells in the absence of INSM1. Nature 563, 691-695, doi: 10.1038/s41586-018-0570-8 (2018).
30 Garcia-Anoveros, J. et al. Tbx2 is a master regulator of inner versus outer hair cell differentiation. Nature 605, 298-303, doi: 10.1038/s41586-022-04668-3 (2022).
31 Christ, Stephanie; Biebel, Ulrich W; Silvi Hoidis; Friedrichsen, Sönke; Bauer, Karl; et al. Audiology & Neurotology; Basel Vol. 9, Iss. 2, (March/April 2004): 88-106.
32 Weber T, Zimmermann U, Winter H, Mack A, Köpschall I, Rohbock K, Zenner H P, Knipper M. Thyroid hormone is a critical determinant for the regulation of the cochlear motor protein prestin. Proc Natl Acad Sci USA. 2002 Mar. 5; 99 (5): 2901-6. doi: 10.1073/pnas.052609899. Epub 2002 Feb. 26. Erratum in: Proc Natl Acad Sci USA 2002 May 28; 99 (11): 7809. PMID: 11867734; PMCID: PMC122445.
31 Ruel, J. et al. Impairment of SLC17A8 encoding vesicular glutamate transporter-3, VGLUT3, underlies nonsyndromic deafness DFNA25 and inner hair cell dysfunction in null mice. Am J Hum Genet 83, 278-292, doi: 10.1016/j.ajhg.2008.07.008 (2008).
32 Dressler, G. R. & Douglass, E. C. Pax-2 Is a DNA-Binding Protein Expressed in Embryonic Kidney and Wilms-Tumor. P Natl Acad Sci USA 89, 1179-1183, doi:DOI 10.1073/pnas.89.4.1179 (1992).
33 Tavassoli, K., Ruger, W. & Horst, J. Alternative splicing in PAX2 generates a new reading frame and an extended conserved coding region at the carboxy terminus. Hum Genet 101, 371-375, doi: DOI 10.1007/s004390050644 (1997).
34 Ward, T. A., Nebel, A., Reeve, A. E. & Eccles, M. R. Alternative Messenger-Rna Forms and Open Reading Frames within an Additional Conserved Region of the Human Pax-2 Gene. Cell Growth Differ 5, 1015-1021 (1994).
35 Gibson, D. G. et al. Enzymatic assembly of DNA molecules up to several hundred kilobases. Nat Methods 6, 343-345, doi: 10.1038/nmeth. 1318 (2009).
36 Hockemeyer, D. et al. Genetic engineering of human pluripotent cells using TALE nucleases. Nat Biotechnol 29, 731-734, doi: 10.1038/nbt. 1927 (2011).
37 Slaymaker, I. M. et al. Rationally engineered Cas9 nucleases with improved specificity. Science 351, 84-88, doi: 10.1126/science.aad5227 (2016).
38 Maruyama, T. et al. Increasing the efficiency of precise genome editing with CRISPR-Cas9 by inhibition of nonhomologous end joining. Nat Biotechnol 33, 538-U260, doi: 10.1038/nbt.3190 (2015).
39 Hama, H. et al. ScaleS: an optical clearing palette for biological imaging. Nat Neurosci 18, 1518-1529, doi: 10.1038/nn.4107 (2015).
40 Hao, Y. et al. Integrated analysis of multimodal single-cell data. Cell 184, 3573-3587 e3529, doi: 10.1016/j.cell.2021.04.048 (2021).
41 Rousseeuw, P. J. Silhouettes—a Graphical Aid to the Interpretation and Validation of Cluster-Analysis. J Comput Appl Math 20, 53-65, doi: Doi 10.1016/0377-0427 (87) 90125-7 (1987).
42 Love, M. I., Huber, W. & Anders, S. Moderated estimation of fold change and dispersion for RNA-seq data with DESeq2. Genome Biol 15, 550, doi: 10.1186/s13059-014-0550-8 (2014).
43 Subramanian, A. et al. Gene set enrichment analysis: a knowledge-based approach for interpreting genome-wide expression profiles. Proc Natl Acad Sci USA 102, 15545-15550, doi: 10.1073/pnas.0506580102 (2005).
44 Kolmykov, S. et al. GTRD: an integrated view of transcription regulation. Nucleic Acids Res 49, D104-D111, doi: 10.1093/nar/gkaa1057 (2021).
45 Locher, H. et al. Neurosensory development and cell fate determination in the human cochlea. Neural Dev 8, 20, doi: 10.1186/1749-8104-8-20 (2013).

We claim:

1. A method of generating human cochlear hair cells comprising:
(a) culturing PAX2b⁺ otic progenitor cells derived from human pluripotent stem cells in medium comprising an activator of sonic hedgehog for about 5 days; and
(b) subsequently culturing the cells of step (a) in medium comprising an activator of sonic hedgehog and a Wnt inhibitor for about 4 days after step (a)
(c) further culturing the cells of step (b) for a sufficient amount of time to differentiate the cells into human cochlear hair cells which express one or more of the otic markers of PRESTIN, NR2F1, GATA3, INSM1, HES6, TMPRSS3 or GNG8.

2. The method of claim 1, wherein the activator of sonic hedgehog in steps (a) and (b) is purmorphamine.

3. The method of claim 2, wherein the concentration of purmorphamine is about 1 nM to about 1 mM.

4. The method of claim 1, wherein the Wnt inhibitor is IWP-2.

5. The method of claim 4, wherein the concentration of IWP-2 is about 1 nM to about 1 mM.

6. The method of claim 1, wherein the otic progenitor cells are cultured with thyroxine for about 50 days, starting about 39 days after the start of step (a).

7. The method of claim 6, wherein the concentration of thyroxine in the medium is about 250 ng/ml.

8. The method of claim 1, wherein a sufficient amount of time is about 89 days after the start of step (a), and wherein the medium does not contain additional agonists or inhibitors about 11 days after the start of step (a).

9. The method of claim 1, wherein a sufficient amount of time is about 139 days after the start of step (a), and wherein the medium does not contain additional agonists or inhibitors about 11 days after the start of step (a).

10. The method of claim 1, wherein the cochlear hair cells express two or markers selected from PRESTIN, NR2F1, GATA3, and INSM1 about 98 days after the start of step (a).

11. The method of claim 10, wherein the cells express PRESTIN, NR2F1, GATA3, and INSM1 about 98 days after the start of step (a).

12. The method of claim 10 or 11, wherein the cells further express one or more additional markers selected from HES6, TMPRSS3 and GNG8 about 98 days after the start of step (a).

13. The method of claim 1, wherein the method results in cells expressing two or more markers selected from PRESTIN, GATA3, INSM1, HES6, TMPRSS3 and GNG8 about 98 days after the start of step (a).

14.-20. (canceled)

21. A method comprising:
(a) culturing pluripotent stem cells in medium comprising FGF-2, BMP-4, and SB431542 for about 3 days on coated plates;
(b) further culturing the cells of step (a) in medium comprising FGF-2, SB431542 and LDN193189 for about 4 days;
(c) further culturing the cells of step (b) in medium comprising CHIR99021, LDN193189, and FGF-2 for about 4 days;
(d) further culturing the cells of step (c) in medium comprising CHIR99021 on coated plates for about 2 days to generate PAX2b⁺ progenitor cells.

22. The method of claim 21, further comprising:
(e) culturing the cells of step (d) in medium comprising CHIR99021 and purmorphamine for about 5 days;
(f) further culturing the cells of step (e) in medium comprising CHIR99021, purmorphamine, and IWP-2 for about 4 days;
(g) further culturing the cells of step (f) in medium for about at least about 78 days to generate human cochlear hair cells.

23. The method of claim 22, wherein the medium in step (g) does not comprise CHIR99021, purmorphamine, or IWP-2.

24.-25. (canceled)

26. A method comprising:
(a) culturing pluripotent stem cells in medium comprising FGF-2, BMP-4, and SB431542 for about 3 days on coated plates;
(b) further culturing the cells of step (a) in medium comprising FGF-2, SB431542 and LDN193189 for about 4 days;
(c) further culturing the cells of step (b) in medium comprising CHIR99021, LDN193189, and FGF-2 for about 4 days;
(d) further culturing the cells of step (c) in medium comprising CHIR99021 on coated plates for about 2 days;
(e) further culturing the cells of step (d) in medium comprising CHIR99021 and purmorphamine for about 5 days;
(f) further culturing the cells of step (e) in medium comprising CHIR99021, purmorphamine, and IWP-2 for about 4 days;
(g) further culturing the cells of step (f) in medium for about 28 days;
(h) further culturing the cells of step (g) in medium comprising thyroxine for about 50 days to generate human cochlear hair cells.

27. The method of claim 26, wherein the medium in step (g) does not comprise CHIR99021, purmorphamine, or IWP-2.

28.-29. (canceled)

30. A kit, platform, or system comprising:
(a) an activator of sonic hedgehog; and
(b) a Wnt inhibitor.

31. The kit, system, or platform of claim 30, wherein the kit, system, or platform further comprises:
(c) FGF-2;
(d) a TGF-beta inhibitor;
(e) a BMP-4 inhibitor; and
(f) a GSK-3 Inhibitor.

32.-33. (canceled)


SEQUENCE LISTING (30 sequences)

SEQ ID NO: 1 - DNA, 53 bp, Homo sapiens
  source 1..53: mol_type = other DNA
ctgctgccgctgcctatgaccgccactagttaccgcggggaccacatcaagct

SEQ ID NO: 2 - DNA, 53 bp, Homo sapiens
  source 1..53: mol_type = other DNA
gacgacggcgacggatactggcggtgatcaatggcgcccctggtgtagttcga

SEQ ID NO: 3 - DNA, 54 bp, Homo sapiens
  source 1..54: mol_type = other DNA
cgaatgaagtattcggctgtccactgattgcggcagggcgcagcgtcgggagcc

SEQ ID NO: 4 - DNA, 54 bp, Homo sapiens
  source 1..54: mol_type = other DNA
gcttacttcataagccgacaggtgactaacgccgtcccgcgtcgcagccctcgg

SEQ ID NO: 5 - DNA, 72 bp, synthetic construct
  source 1..72: mol_type = other DNA
cctgccgctgctgccgctgcctatgaccgccacgctagcgccactaacttctccctgttgaaacaagcaggc

SEQ ID NO: 6 - DNA, 72 bp, synthetic construct
  source 1..72: mol_type = other DNA
gttattaggtccctcgaagaggttcactaggcgcgccttaccgcggggaccacatcaagcttcaggccgaca

SEQ ID NO: 7 - DNA, 22 bp, synthetic construct
  source 1..22: mol_type = other DNA
agacctgcactagctaccgaag

SEQ ID NO: 8 - DNA, 23 bp, synthetic construct
  source 1..23: mol_type = other DNA
acggccgccattagtcaccgagg

SEQ ID NO: 9 - DNA, 23 bp, synthetic construct
  source 1..23: mol_type = other DNA
atgaccaacactagatacagtgg

SEQ ID NO: 10 - DNA, 23 bp, synthetic construct
  source 1..23: mol_type = other DNA
atgacctccacaagttacaaaag

SEQ ID NO: 11 - DNA, 23 bp, synthetic construct
  source 1..23: mol_type = other DNA
aggaccaccactagtaacagagg

SEQ ID NO: 12 - DNA, 23 bp, synthetic construct
  source 1..23: mol_type = other DNA
aggaccaccactagtaacagagg

SEQ ID NO: 13 - DNA, 23 bp, synthetic construct
  source 1..23: mol_type = other DNA
atggccgccacttggtacagagg

SEQ ID NO: 14 - DNA, 23 bp, synthetic construct
  source 1..23: mol_type = other DNA
atgacctccactaggtcctgcag

SEQ ID NO: 15 - DNA, 23 bp, synthetic construct
  source 1..23: mol_type = other DNA
atgacctccactaattccctggg

SEQ ID NO: 16 - DNA, 23 bp, synthetic construct
  source 1..23: mol_type = other DNA
gtgaccgccactagtgatcacgg

SEQ ID NO: 17 - DNA, 73 bp, Homo sapiens
  source 1..73: mol_type = other DNA
aaacagaaacgaatgaagtattcggctgtccacgctagcgccactaacttctccctgttgaaacaagcagggg

SEQ ID NO: 18 - DNA, 72 bp, Homo sapiens
  source 1..72: mol_type = other DNA
actctaggtccctcgaagaggttcactaggcgcgccttgcggcagggcgcagcgtcgggagccgggagagcc

SEQ ID NO: 19 - DNA, 23 bp, synthetic construct
  source 1..23: mol_type = other DNA
gttctgctttccactgattggag

SEQ ID NO: 20 - DNA, 23 bp, synthetic construct
  source 1..23: mol_type = other DNA
agttgtctgtccactgattgtag

SEQ ID NO: 21 - DNA, 23 bp, synthetic construct
  source 1..23: mol_type = other DNA
tctctgctgtccactgatttaag

SEQ ID NO: 22 - DNA, 23 bp, synthetic construct
  source 1..23: mol_type = other DNA
tttctgcggtccactgatttaag

SEQ ID NO: 23 - DNA, 23 bp, synthetic construct
  source 1..23: mol_type = other DNA
agttagctgtccactgattcagg

SEQ ID NO: 24 - DNA, 23 bp, synthetic construct
  source 1..23: mol_type = other DNA
atgcagcagtccactgattacag

SEQ ID NO: 25 - DNA, 23 bp, synthetic construct
  source 1..23: mol_type = other DNA
aatctgctgtccactgagtgggg

SEQ ID NO: 26 - DNA, 23 bp, synthetic construct
  source 1..23: mol_type = other DNA
ggtctgctgtccgctgattgtgg

SEQ ID NO: 27 - DNA, 23 bp, synthetic construct
  source 1..23: mol_type = other DNA
tttctgctggccactggttgaag

SEQ ID NO: 28 - DNA, 23 bp, synthetic construct
  source 1..23: mol_type = other DNA
tttcttatgtccactgattggag

SEQ ID NO: 29 - DNA, 20 bp, synthetic construct
  source 1..20: mol_type = other DNA
atgaccgccactagttaccg

SEQ ID NO: 30 - DNA, 20 bp, synthetic construct
  source 1..20: mol_type = other DNA
attcggctgtccactgattg